SEQUENCE LISTING (281 sequences)

SEQ ID NO: 1 - DNA, 22 bp, synthetic construct
  misc_feature 1..22: note = Synthetic Polynucleotide
  source 1..22: mol_type = other DNA
acgggtgtggtacgcagccact

SEQ ID NO: 2 - DNA, 22 bp, synthetic construct
  misc_feature 1..22: note = Synthetic Polynucleotide
  source 1..22: mol_type = other DNA
ccacttcgagtacccacagtac

SEQ ID NO: 3 - DNA, 22 bp, synthetic construct
  misc_feature 1..22: note = Synthetic Polynucleotide
  source 1..22: mol_type = other DNA
gggtgtggtacgcagccacttc

SEQ ID NO: 4 - DNA, 22 bp, synthetic construct
  misc_feature 1..22: note = Synthetic Polynucleotide
  source 1..22: mol_type = other DNA
gcagccacttcgagtacccaca

SEQ ID NO: 5 - DNA, 22 bp, synthetic construct
  misc_feature 1..22: note = Synthetic Polynucleotide
  source 1..22: mol_type = other DNA
acgggtgtggtacgcagcccct

SEQ ID NO: 6 - protein, 354 aa, synthetic construct
  REGION 1..354: note = Synthetic Polypeptide
MNTKYNKEFLLYLAGFVDGDGSIFARIFKGQHWKFKHYIRLTFTVAQKTQRRWFLDKLVDEIGVGYVTDWGSVSHYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 7 - protein, 354 aa, synthetic construct
  REGION 1..354: note = Synthetic Polypeptide
MNTKYNKEFLLYLAGFVDGDGSIYARIFKGQHWKFKHYIRLTFCVRQKTQRRWFLDKLVDEIGVGYVTDSGSVSAYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 8 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 9 - protein, 354 aa, synthetic construct
  REGION 1..354: note = Synthetic Polypeptide
MNTKYNKEFLLYLAGFVDGDGSIYARIEPRQTAKFKHYLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 10 - protein, 354 aa, synthetic construct
  REGION 1..354: note = Synthetic Polypeptide
MNTKYNKEFLLYLAGFVDGDGSIYARIEPRQTAKFKHYLRLTFHVSQKTQRRWFLDKLVDEIGVGYVVDQGSVSQYFLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYATIMPEQRQKFKHRLRLYFEVCQKTQRRWFLDKLVDEIGVGYVYDQGSVSTYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 11 - protein, 354 aa, synthetic construct
  REGION 1..354: note = Synthetic Polypeptide
MNTKYNKEFLLYLAGFVDGDGSIYARIEPRQNFKFKHTLRLTFFVSQKTQRRWFLDKLVDEIGVGYVTDWGSVSGYYLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYATIIPSQTDKFKHRLRLYFAVSQKTQRRWFLDKLVDEIGVGYVIDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 12 - protein, 354 aa, synthetic construct
  REGION 1..354: note = Synthetic Polypeptide
MNTKYNKEFLLYLAGFVDGDGSIYARIEPRQGNKFKHYLRLTFNVCQKTQRRWFLDKLVDEIGVGYVIDRGSVSDYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYASIQPDQVYKFKHRLRLRFAVFQKTQRRWFLDKLVDEIGVGYVYDHGSVSEYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 13 - protein, 354 aa, synthetic construct
  REGION 1..354: note = Synthetic Polypeptide
MNTKYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYASIEPVQTYKFKHRLRLSFAVSQKTQRRWFLDKLVDEIGVGYVCDVGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 14 - protein, 354 aa, synthetic construct
  REGION 1..354: note = Synthetic Polypeptide
MNTKYNKEFLLYLAGFVDGDGSIYARIEPRQTAKFKHYLRLTFHVSQKTQRRWFLDKLVDEIGVGYVVDQGSVSQYYLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYASITPSQAGKFKHRLRLYFEVSQKTQRRWFLDKLVDEIGVGYVYDQGSVSCYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 15 - protein, 354 aa, synthetic construct
  REGION 1..354: note = Synthetic Polypeptide
MNTKYNKEFLLYLAGFVDGDGSIYARIYPKQSSKFKHHLRLTFHVSQKTQRRWFLDKLVDEIGVGYVYDSGSVSQYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYATIEPAQRYKFKHRLRLKFAVSQKTQRRWFLDKLVDEIGVGYVADQGSVSEYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 16 - protein, 354 aa, synthetic construct
  REGION 1..354: note = Synthetic Polypeptide
MNTKYNKEFLLYLAGFVDGDGSIYARIEPEQTAKFKHMLRLTFRVSQKTQRRWFLDKLVDEIGVGYVNDSGSVSGYYLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYATIEPQQPGKFKHRLSLRFAVYQKTQRRWFLDKLVDEIGVGYVYDPGSVSEYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 17 - protein, 354 aa, synthetic construct
  REGION 1..354: note = Synthetic Polypeptide
MNTKYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYASIGPEQSSKFKHRLRLLFAVAQKTQRRWFLDKLVDEIGVGYVVDQGSVSEYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 18 - protein, 354 aa, synthetic construct
  REGION 1..354: note = Synthetic Polypeptide
MNTKYNKEFLLYLAGFVDGDGSIYARIEPKQTAKFKHHLCLRFHVSQKTQRRWFLDKLVDEIGVGYVVDHGSVSQYFLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYATIQPDQNQKFKHRLRLYFAVYQKTQRRWFLDKLVDEIGVGYVYDTGSVSRYQLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 19 - protein, 354 aa, synthetic construct; identical to SEQ ID NO: 9
  REGION 1..354: note = Synthetic Polypeptide

SEQ ID NO: 20 - protein, 354 aa, synthetic construct
  REGION 1..354: note = Synthetic Polypeptide
MNTKYNKEFLLYLAGFVDGDGSIYARIEPRQRVKFKHFLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 21 - protein, 354 aa, synthetic construct
  REGION 1..354: note = Synthetic Polypeptide
MNTKYNKEFLLYLAGFVDGDGSIYARIEPRQGCKFKHVLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 22 - protein, 354 aa, synthetic construct
  REGION 1..354: note = Synthetic Polypeptide
MNTKYNKEFLLYLAGFVDGDGSIYARIEPRQVCKFKHILRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 23 - protein, 354 aa, synthetic construct
  REGION 1..354: note = Synthetic Polypeptide
MNTKYNKEFLLYLAGFVDGDGSIYARIEPRQCFKFKHCLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 24 - protein, 354 aa, synthetic construct
  REGION 1..354: note = Synthetic Polypeptide
MNTKYNKEFLLYLAGFVDGDGSIYARIEPRQRFKFKHCLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 25 - protein, 354 aa, synthetic construct
  REGION 1..354: note = Synthetic Polypeptide
MNTKYNKEFLLYLAGFVDGDGSIYARIEPRQFCKFKHILRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 26 - protein, 354 aa, synthetic construct
  REGION 1..354: note = Synthetic Polypeptide
MNTKYNKEFLLYLAGFVDGDGSIYARIEPRQAFKFKHALRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 27 - protein, 354 aa, synthetic construct
  REGION 1..354: note = Synthetic Polypeptide
MNTKYNKEFLLYLAGFVDGDGSIYARIEPRQTCKFKHILRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 28 - protein, 354 aa, synthetic construct
  REGION 1..354: note = Synthetic Polypeptide
MNTKYNKEFLLYLAGFVDGDGSIYARIEPRQCFKFKHGLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 29 - protein, 354 aa, synthetic construct
  REGION 1..354: note = Synthetic Polypeptide
MNTKYNKEFLLYLAGFVDGDGSIYARIEPRQSSKFKHLLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 30 - protein, 354 aa, synthetic construct
  REGION 1..354: note = Synthetic Polypeptide
MNTKYNKEFLLYLAGFVDGDGSIYARIYLGQHWKFKHYVRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 31 - protein, 354 aa, synthetic construct
  REGION 1..354: note = Synthetic Polypeptide
MNTKYNKEFLLYLAGFVDGDGSIYARIYLGQHYKFKHYIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 32 - protein, 354 aa, synthetic construct
  REGION 1..354: note = Synthetic Polypeptide
MNTKYNKEFLLYLAGFVDGDGSIYARIYNAQYYKFKHYIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 33 - protein, 354 aa, synthetic construct
  REGION 1..354: note = Synthetic Polypeptide
MNTKYNKEFLLYLAGFVDGDGSIYARIERRQNFKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 34 - protein, 354 aa, synthetic construct
  REGION 1..354: note = Synthetic Polypeptide
MNTKYNKEFLLYLAGFVDGDGSIYARIERRQCVKFKHYLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 35 - protein, 354 aa, synthetic construct
  REGION 1..354: note = Synthetic Polypeptide
MNTKYNKEFLLYLAGFVDGDGSIYARIEYRQGAKFKHYLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 36 - protein, 354 aa, synthetic construct
  REGION 1..354: note = Synthetic Polypeptide
MNTKYNKEFLLYLAGFVDGDGSIYARIEPRQSGKFKHYIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 37 - protein, 354 aa, synthetic construct
  REGION 1..354: note = Synthetic Polypeptide
MNTKYNKEFLLYLAGFVDGDGSIYARIFKGQHWKFKHHIRLTFNVSQKTQRRWFLDKLVDEIGVGYVSDTGSQSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 38 - protein, 354 aa, synthetic construct
  REGION 1..354: note = Synthetic Polypeptide
MNTKYNKEFLLYLAGFVDGDGSIFARIFKGQHWKFKHHIRLTFNVSQKTQRRWFLDKLVDEIGVGYVNDYGSTSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 39 - protein, 354 aa, synthetic construct
  REGION 1..354: note = Synthetic Polypeptide
MNTKYNKEFLLYLAGFVDGDGSIFARIFKGQHWKFKHHIRLTFNVAQKTQRRWFLDKLVDEIGVGYVTDHGSNSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 40 - protein, 354 aa, synthetic construct
  REGION 1..354: note = Synthetic Polypeptide
MNTKYNKEFLLYLAGFVDGDGSIYARIFKGQHWKFKHHIRLTFNVTQKTQRRWFLDKLVDEIGVGYVSDYGSMSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 41 - protein, 354 aa, synthetic construct
  REGION 1..354: note = Synthetic Polypeptide
MNTKYNKEFLLYLAGFVDGDGSIYARIFKGQHWKFKHHIRLTFAVTQKTQRRWFLDKLVDEIGVGYVTDHGSCSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 42 - protein, 354 aa, synthetic construct
  REGION 1..354: note = Synthetic Polypeptide
MNTKYNKEFLLYLAGFVDGDGSIYARIFKGQHWKFKHHIRLTFNVSQKTQRRWFLDKLVDEIGVGYVGDYGSMSHYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 43 - protein, 354 aa, synthetic construct
  REGION 1..354: note = Synthetic Polypeptide
MNTKYNKEFLLYLAGFVDGDGSIYARIFKGQHWKFKHHIRLTFNVSQKTQRRWFLDKLVDEIGVGYVTDFGSQSQYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 44 - protein, 354 aa, synthetic construct
  REGION 1..354: note = Synthetic Polypeptide
MNTKYNKEFLLYLAGFVDGDGSIFARIFKGQHWKFKHHIRLTFNVTQKTQRRWFLDKLVDEIGVGYVSDYGSISNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 45 - protein, 354 aa, synthetic construct
  REGION 1..354: note = Synthetic Polypeptide
MNTKYNKEFLLYLAGFVDGDGSIYARIFKGQHWKFKHHIRLTFNVTQKTQRRWFLDKLVDEIGVGYVIDYGSISNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 46 - protein, 354 aa, synthetic construct
  REGION 1..354: note = Synthetic Polypeptide
MNTKYNKEFLLYLAGFVDGDGSIYARIFKGQHWKFKHHIRLTFNVTQKTQRRWFLDKLVDEIGVGYVTDYGSSSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 47 - protein, 354 aa, synthetic construct
  REGION 1..354: note = Synthetic Polypeptide
MNTKYNKEFLLYLAGFVDGDGSIFARIFKGQHWKFKHHIRLTFNVSQKTQRRWFLDKLVDEIGVGYVTDGGSSSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 48 - protein, 354 aa, synthetic construct
  REGION 1..354: note = Synthetic Polypeptide
MNTKYNKEFLLYLAGFVDGDGSIFARIFKGQHWKFKHHIRLTFNVSQKTQRRWFLDKLVDEIGVGYVYDHGSQSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 49 - protein, 354 aa, synthetic construct
  REGION 1..354: note = Synthetic Polypeptide
MNTKYNKEFLLYLAGFVDGDGSIYARIFKGQHWKFKHHIRLTFNVSQKTQRRWFLDKLVDEIGVGYVCDNGSNSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 50 - protein, 354 aa, synthetic construct
  REGION 1..354: note = Synthetic Polypeptide
MNTKYNKEFLLYLAGFVDGDGSIFARIFKGQHWKFKHHIRLTFNVSQKTQRRWFLDKLVDEIGVGYVADYGSTSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 51 - protein, 354 aa, synthetic construct
  REGION 1..354: note = Synthetic Polypeptide
MNTKYNKEFLLYLAGFVDGDGSIYARIFKGQHWKFKHHIRLTFCVSQKTQRRWFLDKLVDEIGVGYVADYGSMSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 52 - protein, 354 aa, synthetic construct
  REGION 1..354: note = Synthetic Polypeptide
MNTKYNKEFLLYLAGFVDGDGSIMARIFKGQHWKFKHYIRLTLCVRQKTQRRWFLDKLVDEIGVGYVCDSGSMSAYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 53 - protein, 354 aa, synthetic construct; identical to SEQ ID NO: 47
  REGION 1..354: note = Synthetic Polypeptide

SEQ ID NO: 54 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASILPESRMKFKHRLRLGFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 55 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWAQILPEKKCAFKHRLRLWFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 56 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWACIVPEKASRFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 57 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWATIVPEKSSIFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 58 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASIVPEERTRFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 59 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWARIEPEKGGRFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 60 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASISPEVRAPFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 61 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASILPETSARFKHRLRLFFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 62 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWAAILPEQSYRFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 63 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASILPERKTKFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 64 - protein, 354 aa, synthetic construct
  REGION 1..354: note = Synthetic Polypeptide
MNTKYNKEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWACIEPERGYRFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 65 - protein, 354 aa, synthetic construct
  REGION 1..354: note = Synthetic Polypeptide
MNTKYNKEFLLYLAGFVDGDGSIYARIFKGQHWKFKHYIRLTFCVSQKTQRRWFLDKLVDEIGVGYVNDSGSCSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWARIEPELRLRFKHRLRLFFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 66 - protein, 354 aa, synthetic construct
  REGION 1..354: note = Synthetic Polypeptide
MNTKYNKEFLLYLAGFVDGDGSIYARIFKGQHWKFKHYIRLTFCVSQKTQRRWFLDKLVDEIGVGYVNDSGSCSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWAQIVPERAGRFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 67 - protein, 354 aa, synthetic construct
  REGION 1..354: note = Synthetic Polypeptide
MNTKYNKEFLLYLAGFVDGDGSIYARIFKGQHWKFKHHIRLTFNVSQKTQRRWFLDKLVDEIGVGYVCDSGSQSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASITPEPTASFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 68 - protein, 354 aa, synthetic construct
  REGION 1..354: note = Synthetic Polypeptide
MNTKYNKEFLLYLAGFVDGDGSIFARIFKGQHWKFKHHIRLTFCVSQKTQRRWFLDKLVDEIGVGYVVDNGSMSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASINPEPSYRFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 69 - protein, 354 aa, synthetic construct
  REGION 1..354: note = Synthetic Polypeptide
MNTKYNKEFLLYLAGFVDGDGSIYARIFKGQHWKFKHHIRLTFCVTQKTQRRWFLDKLVDEIGVGYVHDYGSCSNYYLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIWASILPEHQYRFKHRLRLGFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 70 - protein, 354 aa, synthetic construct
  REGION 1..354: note = Synthetic Polypeptide
MNTKYNKEFLLYLAGFVDGDGSIYAAIDPDQKYKFKHRLRLIFAVCQKTQRRWFLDKLVDEIGVGYVVDHGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 71 - protein, 354 aa, synthetic construct
  REGION 1..354: note = Synthetic Polypeptide
MNTKYNKEFLLYLAGFVDGDGSIYATIEPAQRYKFKHRLRLKFAVSQKTQRRWFLDKLVDEIGVGYVADQGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 72 - protein, 354 aa, synthetic construct
  REGION 1..354: note = Synthetic Polypeptide
MNTKYNKEFLLYLAGFVDGDGSIYATINPDQTNKFKHRLRLWFAVCQKTQRRWFLDKLVDEIGVGYVADTGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 73 - protein, 354 aa, synthetic construct
  REGION 1..354: note = Synthetic Polypeptide
MNTKYNKEFLLYLAGFVDGDGSIYACIEPAQNSKFKHYLRLRFEVSQKTQRRWFLDKLVDEIGVGYVYDQGSVSSYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 74 - protein, 354 aa, synthetic construct
  REGION 1..354: note = Synthetic Polypeptide
MNTKYNKEFLLYLAGFVDGDGSIYASITPCQSYKFKHRLLLKFSVAQKTQRRWFLDKLVDEIGVGYVTDSGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 75 - protein, 354 aa, synthetic construct
  REGION 1..354: note = Synthetic Polypeptide
MNTKYNKEFLLYLAGFVDGDGSIYATIMPEQTVKFKHRLRLFFEVAQKTQRRWFLDKLVDEIGVGYVYDQGSVSSYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 76 - protein, 354 aa, synthetic construct
  REGION 1..354: note = Synthetic Polypeptide
MNTKYNKEFLLYLAGFVDGDGSIYATIQPEQAPKFKHRLRLHFTVHQKTQRRWFLDKLVDEIGVGYVYDQGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 77 - protein, 354 aa, synthetic construct
  REGION 1..354: note = Synthetic Polypeptide
MNTKYNKEFLLYLAGFVDGDGSIWATIEPSQTLKFKHRLRLHFSVYQKTQRRWFLDKLVDEIGVGYVYDDGSVSRYQLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 78 - protein, 354 aa, synthetic construct
  REGION 1..354: note = Synthetic Polypeptide
MNTKYNKEFLLYLAGFVDGDGSIYASITPCQSYKFKHRLLLKFSVAQKTQRRWFLDKLVDEIGVGYVTDSGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 79 - protein, 354 aa, synthetic construct
  REGION 1..354: note = Synthetic Polypeptide
MNTKYNKEFLLYLAGFVDGDGSIYATIIPSQNLKFKHRLRLYFEVCQKTQRRWFLDKLVDEIGVGYVYDQGSVSSYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 80 - protein, 354 aa, synthetic construct
  REGION 1..354: note = Synthetic Polypeptide
MNTKYNKEFLLYLAGFVDGDGSIYASICPTQAAKFKHRLRLHFAVFQKTQRRWFLDKLVDEIGVGYVTDCGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 81 - protein, 354 aa, synthetic construct
  REGION 1..354: note = Synthetic Polypeptide
MNTKYNKEFLLYLAGFVDGDGSIYASIEPAQSYKFKHRLQLKFAVAQKTQRRWFLDKLVDEIGVGYVCDIGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 82 - protein, 354 aa, synthetic construct
  REGION 1..354: note = Synthetic Polypeptide
MNTKYNKEFLLYLAGFVDGDGSIYAAIMPTQSYKFKHRLRLAFSVSQKTQRRWFLDKLVDEIGVGYVTDAGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 83 - protein, 354 aa, synthetic construct
  REGION 1..354: note = Synthetic Polypeptide
MNTKYNKEFLLYLAGFVDGDGSIYATITPNQRTKFKHQLKLAFAVYQKTQRRWFLDKLVDEIGVGYVYDQGSVSCYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 84 - protein, 354 aa, synthetic construct
  REGION 1..354: note = Synthetic Polypeptide
MNTKYNKEFLLYLAGFVDGDGSIYAAIVPVQSTKFKHRLRLYFTVHQKTQRRWFLDKLVDEIGVGYVYDQGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 85 - protein, 354 aa, synthetic construct
  REGION 1..354: note = Synthetic Polypeptide
MNTKYNKEFLLYLAGFVDGDGSIYATICPVQSLKFKHRLRLFFAVCQKTQRRWFLDKLVDEIGVGYVVDHGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGVSEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 86 - protein, 354 aa, synthetic construct
  REGION 1..354: note = Synthetic Polypeptide
MNTKYNKEFLLYLAGFVDGDGSIWASILPEQRNKFKHRLKLGFSVSQKTQRRWFLDKLVDEIGVGYVVDQGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 87 - protein, 354 aa, synthetic construct
  REGION 1..354: note = Synthetic Polypeptide
MNTKYNKEFLLYLAGFVDGDGSIYATIHPCQCTKFKHRLRLYFEVYQKTQRRWFLDKLVDEIGVGYVYDQGSVSSYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 88 - protein, 354 aa, synthetic construct
  REGION 1..354: note = Synthetic Polypeptide
MNTKYNKEFLLYLAGFVDGDGSIYATIDPDQRQKFKHRLRLKFSVSQKTQRRWFLDKLVDEIGVGYVVDGGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYACIEPKQTSKFKHRLCLRFHVSQKTQRRWFLDKLVDEIGVGYVHDNGSVSQYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 89 - protein, 354 aa, synthetic construct
  REGION 1..354: note = Synthetic Polypeptide
MNTKYNKEFLLYLAGFVDGDGSIYAAIVPDQAPKFKHRLRLFFAVSQKTQRRWFLDKLVDEIGVGYVADQGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIYPGQHYKFKHYLRLTFHVSQKTQRRWFLDKLVDEIGVGYVTDAGSVSQYFLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 90 - protein, 354 aa, synthetic construct
  REGION 1..354: note = Synthetic Polypeptide
MNTKYNKEFLLYLAGFVDGDGSIYASILPEQRNKFKHRLKLSFSVCQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHYLRLTFHVSQKTQRRWFLDKLVDEIGVGYVIDAGSVSGYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 91 - protein, 354 aa, synthetic construct
  REGION 1..354: note = Synthetic Polypeptide
MNTKYNKEFLLYLAGFVDGDGSIYAAIVPDQAPKFKHRLRLFFAVSQKTQRRWFLDKLVDEIGVGYVADQGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYACIEPKQTSKFKHRLCLRFHVSQKTQRRWFLDKLVDEIGVGYVHDNGSVSQYYLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 92 - protein, 354 aa, synthetic construct
  REGION 1..354: note = Synthetic Polypeptide
MNTKYNKEFLLYLAGFVDGDGSIYACICPDQGYKFKHRLTLKFSVAQKTQRRWFLDKLVDEIGVGYVLDQGSVSEYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTAKFKHQLRLTFHVSQKTQRRWFLDKLVDEIGVGYVTDWGSVSGYYLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 93 - protein, 354 aa, synthetic construct
  REGION 1..354: note = Synthetic Polypeptide
MNTKYNKEFLLYLAGFVDGDGSIYACILPEQKTKFKHRLKLSFTVCQKTQRRWFLDKLVDEIGVGYVVDQGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIEPRQTVKFKHMLRLTFHVSQKTQRRWFLDKLVDEIGVGYVHDNGSVSQYYLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 94 - protein, 163 aa, synthetic construct
  REGION 1..163: note = Synthetic Polypeptide
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 95 - protein, 9 aa, synthetic construct
  REGION 1..9: note = Synthetic Polypeptide
LAGLIDADG

SEQ ID NO: 96 - DNA, 1047 bp, synthetic construct
  misc_feature 1..1047: note = Synthetic Polynucleotide
  source 1..1047: mol_type = other DNA
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 97 - DNA, 1047 bp, synthetic construct
  misc_feature 1..1047: note = Synthetic Polynucleotide
  source 1..1047: mol_type = other DNA
atgaatggcacagaaggccctaacttctacgtgcccttctccaatgcgacgggtgtggtacgcagccacttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 98 - DNA, 13706 bp, synthetic construct
  misc_feature 1..13706: note = Synthetic Polynucleotide
  source 1..13706: mol_type = other DNA
gtgggggaccaggagaaagaaagccaaggaagaggaggaggaggaggagaaggaggagaaggatgctgacctagcagctcctctcacagcagctcctctcttgcagaggctgaagagcgatttgtgccctgcaagctaagcccctaatccaccgaggcaaaggcaaagcccctagccgggctcccgagggctgggactcgggtgccccaagatggctgcatccagccatcttggcttagaaagccccccacatgccagcttggccaacacccacaccatgggtttctctggactgcccgacacaaggtgtgggtgctggccaggcctgtgttcaaatcccagctctgcagaggaactttgaccctgcataccccagattcctcagtggtcagtggggagttagaccctcttcatagggggcaggaggagttgttcattcattcaacaaatgtttattgaacacctcctatgggttgtgagctcagaggcagcgatgaacaggccaggctggtcctgcattctagaaatagatgggaagtcagtcaataagtagacaaatgaggccaggtgtggtggcatgcctgtagacccagttactcgggatgctgaggtaggaggatcacttgagcctaggacaggaattcaaggctgcagtaagctatgattgcgccactgcactccagcctgggcaacagagcaagactcatctctaaaaaacatttaaaaattgttttaagaagacaaatgagatagtcgctgatggtaatgactgtgtaaaaactgaacatggctgggtgtggttgctcacacctataatctcagcactttgggaggctgagattacagcctcccaagtggctcccaaggcaggaggatcacttgagcctgggagttagagaacagcttggacaatatagggagagcccaactctacaaaaatgaaaataaattagccaggcatggtggcacacaccaatggtcccagctactcaggggttgaggtggtggaccgcttgagcccaggaggttgaggctgcagtgagccatgatcatgccgctgcactccaacctgagtcacagagtgataccctgtctcaaaaaacaataggccaggtgtggtggctcacgcctgtaaccccagcactttgggaggccgaggcggatggatcacttgagatcaggagttagagaccagcctggctaacatggcaaaatcctgtctgtactaaaaatataaaaattagccaggcatggcagtacatgcctgtagtcctggttacttgggaggctaaggcaggagaatcgcttgaacccaggaagaggaggttgcagtgagccaagatcacaccactgcactccagcttgggtgacagagtgagaccctgtctcaaaacagctaaacctggtgggggtgcctggtgtgtaggatggtcaggggtggtctctccaaggacatgagtgtgagcggagacctgaaggagactcaggaagagattaatactgtcagcaacaaatatattgatcacttacaagcactcccaataatcctattaggtaggcactattatcattcccattttacagagtggagaaccgaagcacactctcgggagggcggggtagctggctgcacccaggctgtgtagcctcagtccagatgtaagggtgggtggaaaagagccttgcccaatgagggagaacagtgaaaccaaggccatagggtctaaagattcacgaaccaggctctcatggagaaagcaggtgaggtttactgtatagatgggtgtgcccctaccccacactgaggcttcctcgtctgagcaaactgaggcccagagaggggaaggaagcaggactaccatggtgactcaaagaccagctagaatccagcctcctctcctcgaggcttccactgccccacgccaggcctgtgtgactcagtctagggcctttccattaccccagctaaacctttctttagtcatttataccatggtgtgaatggctggctggtctttcctgagagctatctttgatgaggggagggaggcatagccaggtttgggaagctgataccccaggaagcccagttgactgtgtgggttatagcccaggctgtcactgatttgtaacgggacctgagcaactctgcagagctaggcctcagtcttttcatctgcaaaatggatatagcagagatggtcagagtaggtgacttcgaatgacccttccagctcactatgagtctgttttcctgaacaaagagcattttttgtttaaaaaaaaatttcttgggccggacacggtggttcactcctataatcctggcactttgggaggccgaggagggtggatcgcttgagccaggagttcaggaccagcctgggcaacatagcgagactccacccctacaaaaaatacaaaaactagtggtgtgcacttgtggtcccagctactcaggaggctgaggtgagaggatcgcttgagcccaggaggcagaggctacagtgagctatgattgtggcactgcactccagcctgggcgacagagaccttgtctcaaaactttttttttcttcgtcaagctttacagaataaagagcactgtcacctcagtgatggctgttagttccccatcaccagggctccatgaggttgcaattgtgaaactcacaaaggaggaacctgagacagagaggggaagtactgagatcatctaggtccattcccccactcactcgttcattcaacaaatattcaggagcaccttctaggtgccaggccctggagacacatcagtgaacaaaacagacatcatcccacctctttccactacaggccaagcaccatgctggtctctgggaaccctgttgtgagcaagacagacccaggcttacccttgtggactcatgttacaggcagggagacgggcacaaaacacaaataaaaagcttccatgctgtcagaagcactatgcaaaaagcaagatgctgaggtactgctaagctgtgtgggatgggggctcagcccggccagggaggggccagttgtgggtcagtcttgacccaaggcatccaggacaccctccttctggccatgagggtccacgtcagaatcaaaccctcaccttaacctcattagcgttgggcataatcaccaggccaagcgccttaaactacgagaggccccatcccacccgccctgccttagccctgccacgtgtgccaaacgctgttagacccaacaccacccaggccaggtagggggctggagcccaggtgggctgcagggaagggggcactcttctgagcagacagatctgggaatcctgggtgggaagagagacagtgagagagagattaagggatatttcccaggcatcagggctttgcactctcaggggtccttccgcctggatgtccttcccctgaagcttcctcctgttgttccgttctcagctcaagctccagcttctcagagaagcctcctgtgttgggagtggctgcgactgaactgtccctactgttattcgctcttctatttgtttgtggtccctgtgccccctcaccccacaaaaacactggcttcttgtgagcaggagcttgctctttcgtgtaccctgtgtgtccccaaggaccaagcaccttgtctgggccacagtaggtgctcaatacacatgttggctggacagtggtcactgagcggccgcacgtcgggcactctcagcacttgcacaggccgccccagacaccccacttcattcctgggaggtgtcatcatgttgcttggacgacggggagagggggacctgccagtgttggcctccattttcccccagtcatctgcccccaaggctctgactactttctttctcacggtacatcctgctattctggaatcggccctcgtggggccacctggtacatggcatttgaggccctcgtggctgattaggcctcccccaacagtgccctgtctgctgcctccagggccagcctccccttcagactggagtcccctgaagggttctgcccctcccctgctctggtagccccctccatcctccctccctccactccatctttgggggcatttgagtcacctttctacaccagtgatctgcccaagccactgctcactttcctctggataaagccaggttccccggcctagcgttcaagacccattacaactgcccccagcccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctctggcatcctctgcctcccctctcagcccctgtcctcaggtgcccctccagcctccctgccgcgttccaagtctcctggtgttgagaaccgcaagcagccgctctgaagcagttcctttttgctttagaataatgtcttgcatttaacaggaaaacagatggggtgctgcagggataacagatcccacttaacagagaggaaaactgaggcagggagaggggaagagactcatttagggatgtggccaggcagcaacaagagcctaggtctcctggctgtgatccaggaatatctctgctgagatgcaggaggagacgctagaagcagccattgcaaagctgggtgacggggagagcttaccgccagccacaagcgtctctctgccagccttgccctgtctcccccatgtccaggctgctgcctcggtcccattctcagggaatctctggccattgttgggtgtttgttgcattcaataatcacagatcactcagttctggccagaaggtgggtgtgccacttacgggtggttgttctctgcagggtcagtcccagtttacaaatattgtccctttcactgttaggaatgtcccagtttggttgattaactatatggccactctccctatggaacttcatggggtggtgagcaggacagatgtctgaattccatcatttccttcttcttcctctgggcaaaacattgcacattgcttcatggctcctaggagaggcccccacatgtccgggttatttcatttcccgagaagggagagggaggaaggactgccaattctgggtttccaccacctctgcattccttcccaacaaggaactctgccccacattaggatgcattcttctgctaaacacacacacacacacacacacacacaacacacacacacacacacacacacacacacacacaaaactccctaccgggttcccagttcaatcctgaccccctgatctgattcgtgtcccttatgggcccagagcgctaagcaaataacttcccccattccctggaatttctttgcccagctctcctcagcgtgtggtccctctgccccttccccctcctcccagcaccaagctctctccttccccaaggcctcctcaaatccctctcccactcctggttgccttcctagctaccctctccctgtctaggggggagtgcaccctccttaggcagtggggtctgtgctgaccgcctgctgactgccttgcaggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtaatggcactgagcagaagggaagaagctccgggggctctttgtagggtcctccagtcaggactcaaacccagtagtgtctggttccaggcactgaccttgtatgtctcctggcccaaatgcccactcagggtaggggtgtagggcagaagaagaaacagactctaatgttgctacaagggctggtcccatctcctgagccccatgtcaaacagaatccaagacatcccaacccttcaccttggctgtgcccctaatcctcaactaagctaggcgcaaattccaatcctctttggtctagtaccccgggggcagccccctctaaccttgggcctcagcagcaggggaggccacaccttcctagtgcaggtggccatattgtggccccttggaactgggtcccactcagcctctaggcgattgtctcctaatggggctgagatgagacacagtggggacagtggtttggacaataggactggtgactctggtccccagaggcctcatgtccctctgtctccagaaaattcccactctcacttccctttcctcctcagtcttgctagggtccatttcttaccccttgctgaatttgagcccaccccctggactttttccccatcttctccaatctggcctagttctatcctctggaagcagagccgctggacgctctgggtttcctgaggcccgtccactgtcaccaatatcaggaaccattgccacgtcctaatgacgtgcgctggaagcctctagtttccagaagctgcacaaagatcccttagatactctgtgtgtccatctttggcctggaaaatactctcaccctggggctaggaagacctcggtttgtacaaacttcctcaaatgcagagcctgagggctctccccacctcctcaccaaccctctgcgtggcatagccctagcctcagcgggcagtggatgctggggctgggcatgcagggagaggctgggtggtgtcatctggtaacgcagccaccaaacaatgaagcgacactgattccacaaggtgcatctgcatccccatctgatccattccatcctgtcacccagccatgcagacgtttatgatccccttttccagggagggaatgtgaagccccagaaagggccagcgctcggcagccaccttggctgttcccaagtccctcacaggcagggtctccctacctgcctgtcctcaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggtacgggccggggggtgggcggcctcacggctctgagggtccagcccccagcatgcatctgcggctcctgctccctggaggagccatggtctggacccgggtcccgtgtcctgcaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcaggtgcctactgcgggtgggagggccccagtgccccaggccacaggcgctgcctgccaaggacaagctacttcccagggcaggggagggggctccatcagggttactggcagcagtcttgggtcagcagtcccaatggggagtgtgtgagaaatgcagattcctggccccactcagaactgctgaatctcagggtgggcccaggaacctgcatttccagcaagccctccacaggtggctcagatgctcactcaggtgggagaagctccagtcagctagttctggaagcccaatgtcaaagtcagaaggacccaagtcgggaatgggatgggccagtctccataaagctgaataaggagctaaaaagtcttattctgaggggtaaaggggtaaagggttcctcggagaggtacctccgaggggtaaacagttgggtaaacagtctctgaagtcagctctgccattttctagctgtatggccctgggcaagtcaatttccttctctgtgctttggtttcctcatccatagaaaggtagaaagggcaaaacaccaaactcttggattacaagagataatttacagaacacccttggcacacagagggcaccatgaaatgtcacgggtgacacagcccccttgtgctcagtccctggcatctctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccggaactgcatgctcaccaccatctgctgcggcaagaacccactgggtgacgatgaggcctctgctaccgtgtccaagacggagacgagccaggtggccccggcctaagacctgcctaggactctgtggccgactataggcgtctcccatcccctacaccttcccccagccacagccatcccaccaggagcagcgcctgtgcagaatgaacgaagtcacataggctccttaattttttttttttttttaagaaataattaatgaggctcctcactcacctgggacagcctgagaagggacatccaccaagacctactgatctggagtcccacgttccccaaggccagcgggatgtgtgcccctcctcctcccaactcatctttcaggaacacgaggattcttgctttctggaaaagtgtcccagcttagggataagtgtctagcacagaatggggcacacagtaggtgcttaataaatgctggatggatgcaggaaggaatggaggaatgaatgggaagggagaacatatctatcctctcagaccctcgcagcagcagcaactcatacttggctaatgatatggagcagttgtttttccctccctgggcctcactttcttctcctataaaatggaaatcccagatccctggtcctgccgacacgcagctactgagaagaccaaaagaggtgtgtgtgtgtctatgtgtgtgtttcagcactttgtaaatagcaagaagctgtacagattctagttaatgttgtgaataacatcaattaatgtaactagttaattactatgattatcacctcctgatagtgaacattttgagattgggcattcagatgatggggtttcacccaaccttggggcaggtttttaaaaattagctaggcatcaaggccagaccagggctgggggttgggctgtaggcagggacagtcacaggaatgcagaatgcagtcatcagacctgaaaaaacaacactgggggagggggacggtgaaggccaagttcccaatgagggtgagattgggcctggggtctcacccctagtgtggggccccaggtcccgtgcctccccttcccaatgtggcctatggagagacaggcctttctctcagcctctggaagccacctgctcttttgctctagcacctgggtcccagcatctagagcatggagcctctagaagccatgctcacccgcccacatttaattaacagctgagtccctgatgtcatccttatctcgaagagcttagaaacaaagagtgggaaattccactgggcctaccttccttggggatgttcatgggccccagtttccagtttcccttgccagacaagcccatcttcagcagttgctagtccattctccattctggagaatctgctccaaaaagctggccacatctctgaggtgtcagaattaagctgcctcagtaactgctcccccttctccatataagcaaagccagaagctctagctttacccagctctgcctggagactaaggcaaattgggccattaaaagctcagctcctatgttggtattaacggtggtgggttttgttgctttcacactctatccacaggatagattgaaactgccagcttccacctgatccctgaccctgggatggctggattgagcaatgagcagagccaagcagcacagagtcccctggggctagaggtggaggaggcagtcctgggaatgggaaaaaccccaactttggggtcatagaggcacaggtaacccataaaactgcaaacaagctttgtcacctctcagagcttccttatctgcaaaaaagaatcttaaaactgaccttggctgggcacagtggctcacacctctaatcccagcactttgggaggccaaggtgggcagatcacgaggtcaggagtttgagaccagcctgaccaacacggtgaaaccctgtctctactaaaaatacaaaaatcagctgggcatggtggcgcgtgcctgtaatcccagctattcagtgggctgaggcaggagaatcgcttgaacctgggaggtggaggttgcagtgagccgagattgcgccactgcactccagcctgagcaacagagggacagtctgtctccaaacaaaacaaaacaaacaaacaaacaaacaaacaaacaaaaaacaacaacaaaaaaaccacttgatcctaaggggattagatgcgactgtggactttaagtggccagcctactgcctggcatgcagcagatgagactatggcaatactgggcttcagctcagagctggccttactagagaccctgtcccaaaggggaaaaggatggagctaaagctcccgagagtcaccccctcctccgaggtgagaaaggagggcaggagcatgagatagccgatcctcggtgccttggtgaggctggggcaaatcatgctgggatctctatcattgtccctctttactgtgactcactagataatatcagtcaggatacttttggtcacaagtgataggaaatccaactcatttgggctgaagcaaaagggacacattgttggctcacatgaacaaaaagcccggggcttcaggcacagggtatcaccatgactgagatggggattaattctgtgattggccaagtctaggtcacctgatcatacgtaactcatttatgcctgaggttgcaattttttggatttttgcaatcagaccttggcgatgaccttgagcagtaggatataaataactcccacatgcttagcgttccaataatggaatactaggcatacgcaggtctaactgcatcaccatggctggaatggggattcatcctctgattggtcagacctaggtcacatgctcaccctgcagcccaagcaggctgaatggggagaggtaggtttcacaaaggaaagcccaggtgctgttacctgaagtaggagggcaggaggcagggtgagcagagccaacatcaacccagagggaatggaatctaagttggtgttttctgggcacgtggctggaccaggcctccctccctcatcatctcagggacatgagggagaagattcctatgggtggtcccgaaggtctcaccctttgttttggatgctgtgttgggccagggtggcagtgggtgggacagtggcatcttagctgccctgacttgcaggcagcccattccagctccccgccccaaccccaacccagcccactttttctgagaaatggtacatttgccccagcctcatgtccagaggaaaattttactctaacaccagaacattctctggtttgtcctgatagacaagaaagcctccacctccttaatttacaaatgacttgacagctgcttcgtgggcacttgcatacataaagagaaggagctgctgccttaagttgcagcaagtttggccccacctcatctccaggcagccagcagatgtacagagtgcctcttgggtacaatggcagctccattcaaccaaacctgagcaagctgaccccatgccagaatgcactggggactcggagatgaattggagcctagagaccaagtctctaggctatgacctgggctgcctcacggccacagagctctgtcacgccaagggagagatgcacccctgaaagcctgaggtgccccataaggagagagtgggtgcccttcccaactatgtagcttcagggcaagttctctttctttctttttctttctttctctttctttctttcttt

SEQ ID NO: 99 - DNA, 13706 bp, synthetic construct
  misc_feature 1..13706: note = Synthetic Polynucleotide
  source 1..13706: mol_type = other DNA
gtgggggaccaggagaaagaaagccaaggaagaggaggaggaggaggagaaggaggagaaggatgctgacctagcagctcctctcacagcagctcctctcttgcagaggctgaagagcgatttgtgccctgcaagctaagcccctaatccaccgaggcaaaggcaaagcccctagccgggctcccgagggctgggactcgggtgccccaagatggctgcatccagccatcttggcttagaaagccccccacatgccagcttggccaacacccacaccatgggtttctctggactgcccgacacaaggtgtgggtgctggccaggcctgtgttcaaatcccagctctgcagaggaactttgaccctgcataccccagattcctcagtggtcagtggggagttagaccctcttcatagggggcaggaggagttgttcattcattcaacaaatgtttattgaacacctcctatgggttgtgagctcagaggcagcgatgaacaggccaggctggtcctgcattctagaaatagatgggaagtcagtcaataagtagacaaatgaggccaggtgtggtggcatgcctgtagacccagttactcgggatgctgaggtaggaggatcacttgagcctaggacaggaattcaaggctgcagtaagctatgattgcgccactgcactccagcctgggcaacagagcaagactcatctctaaaaaacatttaaaaattgttttaagaagacaaatgagatagtcgctgatggtaatgactgtgtaaaaactgaacatggctgggtgtggttgctcacacctataatctcagcactttgggaggctgagattacagcctcccaagtggctcccaaggcaggaggatcacttgagcctgggagttagagaacagcttggacaatatagggagagcccaactctacaaaaatgaaaataaattagccaggcatggtggcacacaccaatggtcccagctactcaggggttgaggtggtggaccgcttgagcccaggaggttgaggctgcagtgagccatgatcatgccgctgcactccaacctgagtcacagagtgataccctgtctcaaaaaacaataggccaggtgtggtggctcacgcctgtaaccccagcactttgggaggccgaggcggatggatcacttgagatcaggagttagagaccagcctggctaacatggcaaaatcctgtctgtactaaaaatataaaaattagccaggcatggcagtacatgcctgtagtcctggttacttgggaggctaaggcaggagaatcgcttgaacccaggaagaggaggttgcagtgagccaagatcacaccactgcactccagcttgggtgacagagtgagaccctgtctcaaaacagctaaacctggtgggggtgcctggtgtgtaggatggtcaggggtggtctctccaaggacatgagtgtgagcggagacctgaaggagactcaggaagagattaatactgtcagcaacaaatatattgatcacttacaagcactcccaataatcctattaggtaggcactattatcattcccattttacagagtggagaaccgaagcacactctcgggagggcggggtagctggctgcacccaggctgtgtagcctcagtccagatgtaagggtgggtggaaaagagccttgcccaatgagggagaacagtgaaaccaaggccatagggtctaaagattcacgaaccaggctctcatggagaaagcaggtgaggtttactgtatagatgggtgtgcccctaccccacactgaggcttcctcgtctgagcaaactgaggcccagagaggggaaggaagcaggactaccatggtgactcaaagaccagctagaatccagcctcctctcctcgaggcttccactgccccacgccaggcctgtgtgactcagtctagggcctttccattaccccagctaaacctttctttagtcatttataccatggtgtgaatggctggctggtctttcctgagagctatctttgatgaggggagggaggcatagccaggtttgggaagctgataccccaggaagcccagttgactgtgtgggttatagcccaggctgtcactgatttgtaacgggacctgagcaactctgcagagctaggcctcagtcttttcatctgcaaaatggatatagcagagatggtcagagtaggtgacttcgaatgacccttccagctcactatgagtctgttttcctgaacaaagagcattttttgtttaaaaaaaaatttcttgggccggacacggtggttcactcctataatcctggcactttgggaggccgaggagggtggatcgcttgagccaggagttcaggaccagcctgggcaacatagcgagactccacccctacaaaaaatacaaaaactagtggtgtgcacttgtggtcccagctactcaggaggctgaggtgagaggatcgcttgagcccaggaggcagaggctacagtgagctatgattgtggcactgcactccagcctgggcgacagagaccttgtctcaaaactttttttttcttcgtcaagctttacagaataaagagcactgtcacctcagtgatggctgttagttccccatcaccagggctccatgaggttgcaattgtgaaactcacaaaggaggaacctgagacagagaggggaagtactgagatcatctaggtccattcccccactcactcgttcattcaacaaatattcaggagcaccttctaggtgccaggccctggagacacatcagtgaacaaaacagacatcatcccacctctttccactacaggccaagcaccatgctggtctctgggaaccctgttgtgagcaagacagacccaggcttacccttgtggactcatgttacaggcagggagacgggcacaaaacacaaataaaaagcttccatgctgtcagaagcactatgcaaaaagcaagatgctgaggtactgctaagctgtgtgggatgggggctcagcccggccagggaggggccagttgtgggtcagtcttgacccaaggcatccaggacaccctccttctggccatgagggtccacgtcagaatcaaaccctcaccttaacctcattagcgttgggcataatcaccaggccaagcgccttaaactacgagaggccccatcccacccgccctgccttagccctgccacgtgtgccaaacgctgttagacccaacaccacccaggccaggtagggggctggagcccaggtgggctgcagggaagggggcactcttctgagcagacagatctgggaatcctgggtgggaagagagacagtgagagagagattaagggatatttcccaggcatcagggctttgcactctcaggggtccttccgcctggatgtccttcccctgaagcttcctcctgttgttccgttctcagctcaagctccagcttctcagagaagcctcctgtgttgggagtggctgcgactgaactgtccctactgttattcgctcttctatttgtttgtggtccctgtgccccctcaccccacaaaaacactggcttcttgtgagcaggagcttgctctttcgtgtaccctgtgtgtccccaaggaccaagcaccttgtctgggccacagtaggtgctcaatacacatgttggctggacagtggtcactgagcggccgcacgtcgggcactctcagcacttgcacaggccgccccagacaccccacttcattcctgggaggtgtcatcatgttgcttggacgacggggagagggggacctgccagtgttggcctccattttcccccagtcatctgcccccaaggctctgactactttctttctcacggtacatcctgctattctggaatcggccctcgtggggccacctggtacatggcatttgaggccctcgtggctgattaggcctcccccaacagtgccctgtctgctgcctccagggccagcctccccttcagactggagtcccctgaagggttctgcccctcccctgctctggtagccccctccatcctccctccctccactccatctttgggggcatttgagtcacctttctacaccagtgatctgcccaagccactgctcactttcctctggataaagccaggttccccggcctagcgttcaagacccattacaactgcccccagcccagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccacttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctctggcatcctctgcctcccctctcagcccctgtcctcaggtgcccctccagcctccctgccgcgttccaagtctcctggtgttgagaaccgcaagcagccgctctgaagcagttcctttttgctttagaataatgtcttgcatttaacaggaaaacagatggggtgctgcagggataacagatcccacttaacagagaggaaaactgaggcagggagaggggaagagactcatttagggatgtggccaggcagcaacaagagcctaggtctcctggctgtgatccaggaatatctctgctgagatgcaggaggagacgctagaagcagccattgcaaagctgggtgacggggagagcttaccgccagccacaagcgtctctctgccagccttgccctgtctcccccatgtccaggctgctgcctcggtcccattctcagggaatctctggccattgttgggtgtttgttgcattcaataatcacagatcactcagttctggccagaaggtgggtgtgccacttacgggtggttgttctctgcagggtcagtcccagtttacaaatattgtccctttcactgttaggaatgtcccagtttggttgattaactatatggccactctccctatggaacttcatggggtggtgagcaggacagatgtctgaattccatcatttccttcttcttcctctgggcaaaacattgcacattgcttcatggctcctaggagaggcccccacatgtccgggttatttcatttcccgagaagggagagggaggaaggactgccaattctgggtttccaccacctctgcattccttcccaacaaggaactctgccccacattaggatgcattcttctgctaaacacacacacacacacacacacacacaacacacacacacacacacacacacacacacacacaaaactccctaccgggttcccagttcaatcctgaccccctgatctgattcgtgtcccttatgggcccagagcgctaagcaaataacttcccccattccctggaatttctttgcccagctctcctcagcgtgtggtccctctgccccttccccctcctcccagcaccaagctctctccttccccaaggcctcctcaaatccctctcccactcctggttgccttcctagctaccctctccctgtctaggggggagtgcaccctccttaggcagtggggtctgtgctgaccgcctgctgactgccttgcaggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtaatggcactgagcagaagggaagaagctccgggggctctttgtagggtcctccagtcaggactcaaacccagtagtgtctggttccaggcactgaccttgtatgtctcctggcccaaatgcccactcagggtaggggtgtagggcagaagaagaaacagactctaatgttgctacaagggctggtcccatctcctgagccccatgtcaaacagaatccaagacatcccaacccttcaccttggctgtgcccctaatcctcaactaagctaggcgcaaattccaatcctctttggtctagtaccccgggggcagccccctctaaccttgggcctcagcagcaggggaggccacaccttcctagtgcaggtggccatattgtggccccttggaactgggtcccactcagcctctaggcgattgtctcctaatggggctgagatgagacacagtggggacagtggtttggacaataggactggtgactctggtccccagaggcctcatgtccctctgtctccagaaaattcccactctcacttccctttcctcctcagtcttgctagggtccatttcttaccccttgctgaatttgagcccaccccctggactttttccccatcttctccaatctggcctagttctatcctctggaagcagagccgctggacgctctgggtttcctgaggcccgtccactgtcaccaatatcaggaaccattgccacgtcctaatgacgtgcgctggaagcctctagtttccagaagctgcacaaagatcccttagatactctgtgtgtccatctttggcctggaaaatactctcaccctggggctaggaagacctcggtttgtacaaacttcctcaaatgcagagcctgagggctctccccacctcctcaccaaccctctgcgtggcatagccctagcctcagcgggcagtggatgctggggctgggcatgcagggagaggctgggtggtgtcatctggtaacgcagccaccaaacaatgaagcgacactgattccacaaggtgcatctgcatccccatctgatccattccatcctgtcacccagccatgcagacgtttatgatccccttttccagggagggaatgtgaagccccagaaagggccagcgctcggcagccaccttggctgttcccaagtccctcacaggcagggtctccctacctgcctgtcctcaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggtacgggccggggggtgggcggcctcacggctctgagggtccagcccccagcatgcatctgcggctcctgctccctggaggagccatggtctggacccgggtcccgtgtcctgcaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcaggtgcctactgcgggtgggagggccccagtgccccaggccacaggcgctgcctgccaaggacaagctacttcccagggcaggggagggggctccatcagggttactggcagcagtcttgggtcagcagtcccaatggggagtgtgtgagaaatgcagattcctggccccactcagaactgctgaatctcagggtgggcccaggaacctgcatttccagcaagccctccacaggtggctcagatgctcactcaggtgggagaagctccagtcagctagttctggaagcccaatgtcaaagtcagaaggacccaagtcgggaatgggatgggccagtctccataaagctgaataaggagctaaaaagtcttattctgaggggtaaaggggtaaagggttcctcggagaggtacctccgaggggtaaacagttgggtaaacagtctctgaagtcagctctgccattttctagctgtatggccctgggcaagtcaatttccttctctgtgctttggtttcctcatccatagaaaggtagaaagggcaaaacaccaaactcttggattacaagagataatttacagaacacccttggcacacagagggcaccatgaaatgtcacgggtgacacagcccccttgtgctcagtccctggcatctctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccggaactgcatgctcaccaccatctgctgcggcaagaacccactgggtgacgatgaggcctctgctaccgtgtccaagacggagacgagccaggtggccccggcctaagacctgcctaggactctgtggccgactataggcgtctcccatcccctacaccttcccccagccacagccatcccaccaggagcagcgcctgtgcagaatgaacgaagtcacataggctccttaattttttttttttttttaagaaataattaatgaggctcctcactcacctgggacagcctgagaagggacatccaccaagacctactgatctggagtcccacgttccccaaggccagcgggatgtgtgcccctcctcctcccaactcatctttcaggaacacgaggattcttgctttctggaaaagtgtcccagcttagggataagtgtctagcacagaatggggcacacagtaggtgcttaataaatgctggatggatgcaggaaggaatggaggaatgaatgggaagggagaacatatctatcctctcagaccctcgcagcagcagcaactcatacttggctaatgatatggagcagttgtttttccctccctgggcctcactttcttctcctataaaatggaaatcccagatccctggtcctgccgacacgcagctactgagaagaccaaaagaggtgtgtgtgtgtctatgtgtgtgtttcagcactttgtaaatagcaagaagctgtacagattctagttaatgttgtgaataacatcaattaatgtaactagttaattactatgattatcacctcctgatagtgaacattttgagattgggcattcagatgatggggtttcacccaaccttggggcaggtttttaaaaattagctaggcatcaaggccagaccagggctgggggttgggctgtaggcagggacagtcacaggaatgcagaatgcagtcatcagacctgaaaaaacaacactgggggagggggacggtgaaggccaagttcccaatgagggtgagattgggcctggggtctcacccctagtgtggggccccaggtcccgtgcctccccttcccaatgtggcctatggagagacaggcctttctctcagcctctggaagccacctgctcttttgctctagcacctgggtcccagcatctagagcatggagcctctagaagccatgctcacccgcccacatttaattaacagctgagtccctgatgtcatccttatctcgaagagcttagaaacaaagagtgggaaattccactgggcctaccttccttggggatgttcatgggccccagtttccagtttcccttgccagacaagcccatcttcagcagttgctagtccattctccattctggagaatctgctccaaaaagctggccacatctctgaggtgtcagaattaagctgcctcagtaactgctcccccttctccatataagcaaagccagaagctctagctttacccagctctgcctggagactaaggcaaattgggccattaaaagctcagctcctatgttggtattaacggtggtgggttttgttgctttcacactctatccacaggatagattgaaactgccagcttccacctgatccctgaccctgggatggctggattgagcaatgagcagagccaagcagcacagagtcccctggggctagaggtggaggaggcagtcctgggaatgggaaaaaccccaactttggggtcatagaggcacaggtaacccataaaactgcaaacaagctttgtcacctctcagagcttccttatctgcaaaaaagaatcttaaaactgaccttggctgggcacagtggctcacacctctaatcccagcactttgggaggccaaggtgggcagatcacgaggtcaggagtttgagaccagcctgaccaacacggtgaaaccctgtctctactaaaaatacaaaaatcagctgggcatggtggcgcgtgcctgtaatcccagctattcagtgggctgaggcaggagaatcgcttgaacctgggaggtggaggttgcagtgagccgagattgcgccactgcactccagcctgagcaacagagggacagtctgtctccaaacaaaacaaaacaaacaaacaaacaaacaaacaaacaaaaaacaacaacaaaaaaaccacttgatcctaaggggattagatgcgactgtggactttaagtggccagcctactgcctggcatgcagcagatgagactatggcaatactgggcttcagctcagagctggccttactagagaccctgtcccaaaggggaaaaggatggagctaaagctcccgagagtcaccccctcctccgaggtgagaaaggagggcaggagcatgagatagccgatcctcggtgccttggtgaggctggggcaaatcatgctgggatctctatcattgtccctctttactgtgactcactagataatatcagtcaggatacttttggtcacaagtgataggaaatccaactcatttgggctgaagcaaaagggacacattgttggctcacatgaacaaaaagcccggggcttcaggcacagggtatcaccatgactgagatggggattaattctgtgattggccaagtctaggtcacctgatcatacgtaactcatttatgcctgaggttgcaattttttggatttttgcaatcagaccttggcgatgaccttgagcagtaggatataaataactcccacatgcttagcgttccaataatggaatactaggcatacgcaggtctaactgcatcaccatggctggaatggggattcatcctctgattggtcagacctaggtcacatgctcaccctgcagcccaagcaggctgaatggggagaggtaggtttcacaaaggaaagcccaggtgctgttacctgaagtaggagggcaggaggcagggtgagcagagccaacatcaacccagagggaatggaatctaagttggtgttttctgggcacgtggctggaccaggcctccctccctcatcatctcagggacatgagggagaagattcctatgggtggtcccgaaggtctcaccctttgttttggatgctgtgttgggccagggtggcagtgggtgggacagtggcatcttagctgccctgacttgcaggcagcccattccagctccccgccccaaccccaacccagcccactttttctgagaaatggtacatttgccccagcctcatgtccagaggaaaattttactctaacaccagaacattctctggtttgtcctgatagacaagaaagcctccacctccttaatttacaaatgacttgacagctgcttcgtgggcacttgcatacataaagagaaggagctgctgccttaagttgcagcaagtttggccccacctcatctccaggcagccagcagatgtacagagtgcctcttgggtacaatggcagctccattcaaccaaacctgagcaagctgaccccatgccagaatgcactggggactcggagatgaattggagcctagagaccaagtctctaggctatgacctgggctgcctcacggccacagagctctgtcacgccaagggagagatgcacccctgaaagcctgaggtgccccataaggagagagtgggtgcccttcccaactatgtagcttcagggcaagttctctttctttctttttctttctttctctttctttctttcttt

SEQ ID NO: 100 - protein, 348 aa, synthetic construct [sequence omitted]

SEQ ID NO: 101 - protein, 348 aa, synthetic construct
  REGION 1..348: note = Synthetic Polypeptide
MNGTEGPNFYVPFSNATGVVRSHFEYPQYYLAEPWQFSMLAAYMFLLIVLGFPINFLTLYVTVQHKKLRTPLNYILLNLAVADLFMVLGGFTSTLYTSLHGYFVFGPTGCNLEGFFATLGGEIALWSLVVLAIERYVVVCKPMSNFRFGENHAIMGVAFTWVMALACAAPPLAGWSRYIPEGLQCSCGIDYYTLKPEVNNESFVIYMFVVHFTIPMIIIFFCYGQLVFTVKEAAAQQQESATTQKAEKEVTRMVIIMVIAFLICWVPYASVAFYIFTHQGSNFGPIFMTIPAFFAKSAAIYNPVIYIMMNKQFRNCMLTTICCGKNPLGDDEASATVSKTETSQVAPA

SEQ ID NO: 102 - protein, 147 aa, synthetic construct
  REGION 1..147: note = Synthetic Polypeptide
KEFLLYLAGFVDGDGSIWASIIPEQGYKFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 103 - protein, 147 aa, synthetic construct; identical to SEQ ID NO: 102
  REGION 1..147: note = Synthetic Polypeptide

SEQ ID NO: 104 - protein, 147 aa, synthetic construct; identical to SEQ ID NO: 102
  REGION 1..147: note = Synthetic Polypeptide

SEQ ID NO: 105 - protein, 147 aa, synthetic construct; identical to SEQ ID NO: 102
  REGION 1..147: note = Synthetic Polypeptide

SEQ ID NO: 106 - protein, 147 aa, synthetic construct
  REGION 1..147: note = Synthetic Polypeptide
KEFLLYLAGFVDGDGSIYATIMPEQRQKFKHRLRLYFEVCQKTQRRWFLDKLVDEIGVGYVYDQGSVSTYRLSQIKPLHNFLTQLQPFLKLKQKQANLVLKIIEQLPSAKESPDKFLEVCTWVDQIAALNDSKTRKTTSETVRAVLD

SEQ ID NO: 107 - protein, 147 aa, synthetic construct
  REGION 1..147: note = Synthetic Polypeptide
KEFLLYLAGFVDGDGSIYATIIPSQTDKFKHRLRLYFAVSQKTQRRWFLDKLVDEIGVGYVIDSGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 108 - protein, 147 aa, synthetic construct
  REGION 1..147: note = Synthetic Polypeptide
KEFLLYLAGFVDGDGSIYASIQPDQVYKFKHRLRLRFAVFQKTQRRWFLDKLVDEIGVGYVYDHGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 109 - protein, 147 aa, synthetic construct
  REGION 1..147: note = Synthetic Polypeptide
KEFLLYLAGFVDGDGSIYASIEPVQTYKFKHRLRLSFAVSQKTQRRWFLDKLVDEIGVGYVCDVGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 110 - protein, 147 aa, synthetic construct
  REGION 1..147: note = Synthetic Polypeptide
KEFLLYLAGFVDGDGSIYASITPSQAGKFKHRLRLYFEVSQKTQRRWFLDKLVDEIGVGYVYDQGSVSCYRLSQIKPLHNFLTQLQPFLKLKQKQANLVLKIIEQLPSAKESPDKFLEVCTWVDQIAALNDSKTRKTTSETVRAVLD

SEQ ID NO: 111 - protein, 147 aa, synthetic construct
  REGION 1..147: note = Synthetic Polypeptide
KEFLLYLAGFVDGDGSIYATIEPAQRYKFKHRLRLKFAVSQKTQRRWFLDKLVDEIGVGYVADQGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 112 - protein, 147 aa, synthetic construct
  REGION 1..147: note = Synthetic Polypeptide
KEFLLYLAGFVDGDGSIYATIEPQQPGKFKHRLSLRFAVYQKTQRRWFLDKLVDEIGVGYVYDPGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 113 - protein, 147 aa, synthetic construct
  REGION 1..147: note = Synthetic Polypeptide
KEFLLYLAGFVDGDGSIYASIGPEQSSKFKHRLRLLFAVAQKTQRRWFLDKLVDEIGVGYVVDQGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 114 - protein, 147 aa, synthetic construct
  REGION 1..147: note = Synthetic Polypeptide
KEFLLYLAGFVDGDGSIYATIQPDQNQKFKHRLRLYFAVYQKTQRRWFLDKLVDEIGVGYVYDTGSVSRYQLSQIKPLHNFLTQLQPFLKLKQKQANLVLKIIEQLPSAKESPDKFLEVCTWVDQIAALNDSKTRKTTSETVRAVLD

SEQ ID NO: 115 - protein, 147 aa, synthetic construct; identical to SEQ ID NO: 102
  REGION 1..147: note = Synthetic Polypeptide

SEQ ID NO: 116 - protein, 147 aa, synthetic construct; identical to SEQ ID NO: 102
  REGION 1..147: note = Synthetic Polypeptide

SEQ ID NO: 117 - protein, 147 aa, synthetic construct; identical to SEQ ID NO: 102
  REGION 1..147: note = Synthetic Polypeptide

SEQ ID NO: 118 - protein, 147 aa, synthetic construct; identical to SEQ ID NO: 102
  REGION 1..147: note = Synthetic Polypeptide

SEQ ID NO: 119 - protein, 147 aa, synthetic construct; identical to SEQ ID NO: 102
  REGION 1..147: note = Synthetic Polypeptide

SEQ ID NO: 120 - protein, 147 aa, synthetic construct; identical to SEQ ID NO: 102
  REGION 1..147: note = Synthetic Polypeptide

SEQ ID NO: 121 - protein, 147 aa, synthetic construct; identical to SEQ ID NO: 102
  REGION 1..147: note = Synthetic Polypeptide

SEQ ID NO: 122 - protein, 147 aa, synthetic construct; identical to SEQ ID NO: 102
  REGION 1..147: note = Synthetic Polypeptide

SEQ ID NO: 123 - protein, 147 aa, synthetic construct; identical to SEQ ID NO: 102
  REGION 1..147: note = Synthetic Polypeptide

SEQ ID NO: 124 - protein, 147 aa, synthetic construct; identical to SEQ ID NO: 102
  REGION 1..147: note = Synthetic Polypeptide

SEQ ID NO: 125 - protein, 147 aa, synthetic construct; identical to SEQ ID NO: 102
  REGION 1..147: note = Synthetic Polypeptide

SEQ ID NO: 126 - protein, 147 aa, synthetic construct; identical to SEQ ID NO: 102
  REGION 1..147: note = Synthetic Polypeptide

SEQ ID NO: 127 - protein, 147 aa, synthetic construct; identical to SEQ ID NO: 102
  REGION 1..147: note = Synthetic Polypeptide

SEQ ID NO: 128 - protein, 147 aa, synthetic construct; identical to SEQ ID NO: 102
  REGION 1..147: note = Synthetic Polypeptide

SEQ ID NO: 129 - protein, 147 aa, synthetic construct; identical to SEQ ID NO: 102
  REGION 1..147: note = Synthetic Polypeptide

SEQ ID NO: 130 - protein, 147 aa, synthetic construct; identical to SEQ ID NO: 102
  REGION 1..147: note = Synthetic Polypeptide

SEQ ID NO: 131 - protein, 147 aa, synthetic construct; identical to SEQ ID NO: 102
  REGION 1..147: note = Synthetic Polypeptide

SEQ ID NO: 132 - protein, 147 aa, synthetic construct; identical to SEQ ID NO: 102
  REGION 1..147: note = Synthetic Polypeptide

SEQ ID NO: 133 - protein, 147 aa, synthetic construct; identical to SEQ ID NO: 102
  REGION 1..147: note = Synthetic Polypeptide

SEQ ID NO: 134 - protein, 147 aa, synthetic construct; identical to SEQ ID NO: 102
  REGION 1..147: note = Synthetic Polypeptide

SEQ ID NO: 135 - protein, 147 aa, synthetic construct; identical to SEQ ID NO: 102
  REGION 1..147: note = Synthetic Polypeptide

SEQ ID NO: 136 - protein, 147 aa, synthetic construct; identical to SEQ ID NO: 102
  REGION 1..147: note = Synthetic Polypeptide

SEQ ID NO: 137 - protein, 147 aa, synthetic construct; identical to SEQ ID NO: 102
  REGION 1..147: note = Synthetic Polypeptide

SEQ ID NO: 138 - protein, 147 aa, synthetic construct; identical to SEQ ID NO: 102
  REGION 1..147: note = Synthetic Polypeptide

SEQ ID NO: 139 - protein, 147 aa, synthetic construct; identical to SEQ ID NO: 102
  REGION 1..147: note = Synthetic Polypeptide

SEQ ID NO: 140 - protein, 147 aa, synthetic construct; identical to SEQ ID NO: 102
  REGION 1..147: note = Synthetic Polypeptide

SEQ ID NO: 141 - protein, 147 aa, synthetic construct; identical to SEQ ID NO: 102
  REGION 1..147: note = Synthetic Polypeptide

SEQ ID NO: 142 - protein, 147 aa, synthetic construct; identical to SEQ ID NO: 102
  REGION 1..147: note = Synthetic Polypeptide

SEQ ID NO: 143 - protein, 147 aa, synthetic construct; identical to SEQ ID NO: 102
  REGION 1..147: note = Synthetic Polypeptide

SEQ ID NO: 144 - protein, 147 aa, synthetic construct; identical to SEQ ID NO: 102
  REGION 1..147: note = Synthetic Polypeptide

SEQ ID NO: 145 - protein, 147 aa, synthetic construct; identical to SEQ ID NO: 102
  REGION 1..147: note = Synthetic Polypeptide

SEQ ID NO: 146 - protein, 147 aa, synthetic construct; identical to SEQ ID NO: 102
  REGION 1..147: note = Synthetic Polypeptide

SEQ ID NO: 147 - protein, 147 aa, synthetic construct; identical to SEQ ID NO: 102
  REGION 1..147: note = Synthetic Polypeptide

SEQ ID NO: 148 - protein, 147 aa, synthetic construct; identical to SEQ ID NO: 102
  REGION 1..147: note = Synthetic Polypeptide

SEQ ID NO: 149 - protein, 147 aa, synthetic construct; identical to SEQ ID NO: 102
  REGION 1..147: note = Synthetic Polypeptide

SEQ ID NO: 150 - protein, 147 aa, synthetic construct
  REGION 1..147: note = Synthetic Polypeptide
KEFLLYLAGFVDGDGSIWASILPESRMKFKHRLRLGFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 151 - protein, 147 aa, synthetic construct
  REGION 1..147: note = Synthetic Polypeptide
KEFLLYLAGFVDGDGSIWAQILPEKKCAFKHRLRLWFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 152 - protein, 147 aa, synthetic construct
  REGION 1..147: note = Synthetic Polypeptide
KEFLLYLAGFVDGDGSIWACIVPEKASRFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 153 - protein, 147 aa, synthetic construct
  REGION 1..147: note = Synthetic Polypeptide
KEFLLYLAGFVDGDGSIWATIVPEKSSIFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 154 - protein, 147 aa, synthetic construct
  REGION 1..147: note = Synthetic Polypeptide
KEFLLYLAGFVDGDGSIWASIVPEERTRFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 155 - protein, 147 aa, synthetic construct
  REGION 1..147: note = Synthetic Polypeptide
KEFLLYLAGFVDGDGSIWARIEPEKGGRFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 156 - protein, 147 aa, synthetic construct
  REGION 1..147: note = Synthetic Polypeptide
KEFLLYLAGFVDGDGSIWASISPEVRAPFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 157 - protein, 147 aa, synthetic construct
  REGION 1..147: note = Synthetic Polypeptide
KEFLLYLAGFVDGDGSIWASILPETSARFKHRLRLFFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 158 - protein, 147 aa, synthetic construct
  REGION 1..147: note = Synthetic Polypeptide
KEFLLYLAGFVDGDGSIWAAILPEQSYRFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 159 - protein, 147 aa, synthetic construct
  REGION 1..147: note = Synthetic Polypeptide
KEFLLYLAGFVDGDGSIWASILPERKTKFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 160 - protein, 147 aa, synthetic construct
  REGION 1..147: note = Synthetic Polypeptide
KEFLLYLAGFVDGDGSIWACIEPERGYRFKHRLRLAFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 161 - protein, 147 aa, synthetic construct
  REGION 1..147: note = Synthetic Polypeptide
KEFLLYLAGFVDGDGSIWARIEPELRLRFKHRLRLFFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 162 - protein, 147 aa, synthetic construct
  REGION 1..147: note = Synthetic Polypeptide
KEFLLYLAGFVDGDGSIWAQIVPERAGRFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 163 - protein, 147 aa, synthetic construct
  REGION 1..147: note = Synthetic Polypeptide
KEFLLYLAGFVDGDGSIWASITPEPTASFKHRLRLYFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 164 - protein, 147 aa, synthetic construct
  REGION 1..147: note = Synthetic Polypeptide
KEFLLYLAGFVDGDGSIWASINPEPSYRFKHRLRLSFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 165 - protein, 147 aa, synthetic construct
  REGION 1..147: note = Synthetic Polypeptide
KEFLLYLAGFVDGDGSIWASILPEHQYRFKHRLRLGFTVAQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 166 - protein, 147 aa, synthetic construct
  REGION 1..147: note = Synthetic Polypeptide
KEFLLYLAGFVDGDGSIYAAIDPDQKYKFKHRLRLIFAVCQKTQRRWFLDKLVDEIGVGYVVDHGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 167 - protein, 147 aa, synthetic construct
  REGION 1..147: note = Synthetic Polypeptide
KEFLLYLAGFVDGDGSIYATIEPAQRYKFKHRLRLKFAVSQKTQRRWFLDKLVDEIGVGYVADQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 168 - protein, 147 aa, synthetic construct
  REGION 1..147: note = Synthetic Polypeptide
KEFLLYLAGFVDGDGSIYATINPDQTNKFKHRLRLWFAVCQKTQRRWFLDKLVDEIGVGYVADTGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 169 - protein, 147 aa, synthetic construct
  REGION 1..147: note = Synthetic Polypeptide
KEFLLYLAGFVDGDGSIYACIEPAQNSKFKHYLRLRFEVSQKTQRRWFLDKLVDEIGVGYVYDQGSVSSYRLSQIKPLHNFLTQLQPFLKLKQKQANLVLKIIEQLPSAKESPDKFLEVCTWVDQIAALNDSKTRKTTSETVRAVLD

SEQ ID NO: 170 - protein, 147 aa, synthetic construct
  REGION 1..147: note = Synthetic Polypeptide
KEFLLYLAGFVDGDGSIYASITPCQSYKFKHRLLLKFSVAQKTQRRWFLDKLVDEIGVGYVTDSGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 171 - protein, 147 aa, synthetic construct
  REGION 1..147: note = Synthetic Polypeptide
KEFLLYLAGFVDGDGSIYATIMPEQTVKFKHRLRLFFEVAQKTQRRWFLDKLVDEIGVGYVYDQGSVSSYRLSQIKPLHNFLTQLQPFLKLKQKQANLVLKIIEQLPSAKESPDKFLEVCTWVDQIAALNDSKTRKTTSETVRAVLD

SEQ ID NO: 172 - protein, 147 aa, synthetic construct
  REGION 1..147: note = Synthetic Polypeptide
KEFLLYLAGFVDGDGSIYATIQPEQAPKFKHRLRLHFTVHQKTQRRWFLDKLVDEIGVGYVYDQGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 173 - protein, 147 aa, synthetic construct
  REGION 1..147: note = Synthetic Polypeptide
KEFLLYLAGFVDGDGSIWATIEPSQTLKFKHRLRLHFSVYQKTQRRWFLDKLVDEIGVGYVYDDGSVSRYQLSQIKPLHNFLTQLQPFLKLKQKQANLVLKIIEQLPSAKESPDKFLEVCTWVDQIAALNDSKTRKTTSETVRAVLD

SEQ ID NO: 174 - protein, 147 aa, synthetic construct
  REGION 1..147: note = Synthetic Polypeptide
KEFLLYLAGFVDGDGSIYASITPCQSYKFKHRLLLKFSVAQKTQRRWFLDKLVDEIGVGYVTDSGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 175 - protein, 147 aa, synthetic construct
  REGION 1..147: note = Synthetic Polypeptide
KEFLLYLAGFVDGDGSIYATIIPSQNLKFKHRLRLYFEVCQKTQRRWFLDKLVDEIGVGYVYDQGSVSSYRLSEIKPLHNFLTQLQPFLKLKQKQANLVLKIIEQLPSAKESPDKFLEVCTWVDQIAALNDSKTRKTTSETVRAVLD

SEQ ID NO: 176 - protein, 147 aa, synthetic construct
  REGION 1..147: note = Synthetic Polypeptide
KEFLLYLAGFVDGDGSIYASICPTQAAKFKHRLRLHFAVFQKTQRRWFLDKLVDEIGVGYVTDCGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 177 - protein, 147 aa, synthetic construct
  REGION 1..147: note = Synthetic Polypeptide
KEFLLYLAGFVDGDGSIYASIEPAQSYKFKHRLQLKFAVAQKTQRRWFLDKLVDEIGVGYVCDIGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 178 - protein, 147 aa, synthetic construct
  REGION 1..147: note = Synthetic Polypeptide
KEFLLYLAGFVDGDGSIYAAIMPTQSYKFKHRLRLAFSVSQKTQRRWFLDKLVDEIGVGYVTDAGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 179 - protein, 147 aa, synthetic construct
  REGION 1..147: note = Synthetic Polypeptide
KEFLLYLAGFVDGDGSIYATITPNQRTKFKHQLKLAFAVYQKTQRRWFLDKLVDEIGVGYVYDQGSVSCYRLSEIKPLHNFLTQLQPFLKLKQKQANLVLKIIEQLPSAKESPDKFLEVCTWVDQIAALNDSKTRKTTSETVRAVLD

SEQ ID NO: 180 - protein, 147 aa, synthetic construct
  REGION 1..147: note = Synthetic Polypeptide
KEFLLYLAGFVDGDGSIYAAIVPVQSTKFKHRLRLYFTVHQKTQRRWFLDKLVDEIGVGYVYDQGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 181 - protein, 147 aa, synthetic construct
  REGION 1..147: note = Synthetic Polypeptide
KEFLLYLAGFVDGDGSIYATICPVQSLKFKHRLRLFFAVCQKTQRRWFLDKLVDEIGVGYVVDHGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 182 - protein, 147 aa, synthetic construct
  REGION 1..147: note = Synthetic Polypeptide
KEFLLYLAGFVDGDGSIWASILPEQRNKFKHRLKLGFSVSQKTQRRWFLDKLVDEIGVGYVVDQGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 183 - protein, 147 aa, synthetic construct
  REGION 1..147: note = Synthetic Polypeptide
KEFLLYLAGFVDGDGSIYATIHPCQCTKFKHRLRLYFEVYQKTQRRWFLDKLVDEIGVGYVYDQGSVSSYRLSQIKPLHNFLTQLQPFLKLKQKQANLVLKIIEQLPSAKESPDKFLEVCTWVDQIAALNDSKTRKTTSETVRAVLD

SEQ ID NO: 184 - protein, 147 aa, synthetic construct
  REGION 1..147: note = Synthetic Polypeptide
KEFLLYLAGFVDGDGSIYATIDPDQRQKFKHRLRLKFSVSQKTQRRWFLDKLVDEIGVGYVVDGGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 185 - protein, 147 aa, synthetic construct
  REGION 1..147: note = Synthetic Polypeptide
KEFLLYLAGFVDGDGSIYAAIVPDQAPKFKHRLRLFFAVSQKTQRRWFLDKLVDEIGVGYVADQGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 186 - protein, 147 aa, synthetic construct
  REGION 1..147: note = Synthetic Polypeptide
KEFLLYLAGFVDGDGSIYASILPEQRNKFKHRLKLSFSVCQKTQRRWFLDKLVDEIGVGYVVDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 187 - protein, 147 aa, synthetic construct
  REGION 1..147: note = Synthetic Polypeptide
KEFLLYLAGFVDGDGSIYAAIVPDQAPKFKHRLRLFFAVSQKTQRRWFLDKLVDEIGVGYVADQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 188 - protein, 147 aa, synthetic construct
  REGION 1..147: note = Synthetic Polypeptide
KEFLLYLAGFVDGDGSIYACICPDQGYKFKHRLTLKFSVAQKTQRRWFLDKLVDEIGVGYVLDQGSVSEYRLSEIKPLHNFLTQLQPFLKLKQKQANLVLKIIEQLPSAKESPDKFLEVCTWVDQIAALNDSKTRKTTSETVRAVLD

SEQ ID NO: 189 - protein, 147 aa, synthetic construct
  REGION 1..147: note = Synthetic Polypeptide
KEFLLYLAGFVDGDGSIYACILPEQKTKFKHRLKLSFTVCQKTQRRWFLDKLVDEIGVGYVVDQGSVSEYRLSQIKPLHNFLTQLQPFLKLKQKQANLVLKIIEQLPSAKESPDKFLEVCTWVDQIAALNDSKTRKTTSETVRAVLD

SEQ ID NO: 190 - protein, 147 aa, synthetic construct
  REGION 1..147: note = Synthetic Polypeptide
KEFLLYLAGFVDGDGSIFARIFKGQHWKFKHYIRLTFTVAQKTQRRWFLDKLVDEIGVGYVTDWGSVSHYYLSEIKPLHNFLTQLQPFLKLKQKQANLVLKIIEQLPSAKESPDKFLEVCTWVDQIAALNDSKTRKTTSETVRAVLD

SEQ ID NO: 191 - protein, 147 aa, synthetic construct
  REGION 1..147: note = Synthetic Polypeptide
KEFLLYLAGFVDGDGSIYARIFKGQHWKFKHYIRLTFCVRQKTQRRWFLDKLVDEIGVGYVTDSGSVSAYYLSEIKPLHNFLTQLQPFLKLKQKQANLVLKIIEQLPSAKESPDKFLEVCTWVDQIAALNDSKTRKTTSETVRAVLD

SEQ ID NO: 192 - protein, 147 aa, synthetic construct
  REGION 1..147: note = Synthetic Polypeptide
KEFLLYLAGFVDGDGSIYARIFKGQHWKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 193 - protein, 147 aa, synthetic construct
  REGION 1..147: note = Synthetic Polypeptide
KEFLLYLAGFVDGDGSIYARIEPRQTAKFKHYLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 194 - protein, 147 aa, synthetic construct
  REGION 1..147: note = Synthetic Polypeptide
KEFLLYLAGFVDGDGSIYARIEPRQTAKFKHYLRLTFHVSQKTQRRWFLDKLVDEIGVGYVVDQGSVSQYFLSEIKPLHNFLTQLQPFLKLKQKQANLVLKIIEQLPSAKESPDKFLEVCTWVDQIAALNDSKTRKTTSETVRAVLD

SEQ ID NO: 195 - protein, 147 aa, synthetic construct
  REGION 1..147: note = Synthetic Polypeptide
KEFLLYLAGFVDGDGSIYARIEPRQNFKFKHTLRLTFFVSQKTQRRWFLDKLVDEIGVGYVTDWGSVSGYYLSQIKPLHNFLTQLQPFLKLKQKQANLVLKIIEQLPSAKESPDKFLEVCTWVDQIAALNDSKTRKTTSETVRAVLD

SEQ ID NO: 196 - protein, 147 aa, synthetic construct
  REGION 1..147: note = Synthetic Polypeptide
KEFLLYLAGFVDGDGSIYARIEPRQGNKFKHYLRLTFNVCQKTQRRWFLDKLVDEIGVGYVIDRGSVSDYYLSEIKPLHNFLTQLQPFLKLKQKQANLVLKIIEQLPSAKESPDKFLEVCTWVDQIAALNDSKTRKTTSETVRAVLD

SEQ ID NO: 197 - protein, 147 aa, synthetic construct
  REGION 1..147: note = Synthetic Polypeptide
KEFLLYLAGFVDGDGSIYARIEPRQTAKFKHYLRLTFHVSQKTQRRWFLDKLVDEIGVGYVVDQGSVSQYFLSQIKPLHNFLTQLQPFLKLKQKQANLVLKIIEQLPSAKESPDKFLEVCTWVDQIAALNDSKTRKTTSETVRAVLD

SEQ ID NO: 198 - protein, 147 aa, synthetic construct
  REGION 1..147: note = Synthetic Polypeptide
KEFLLYLAGFVDGDGSIYARIEPRQTAKFKHYLRLTFHVSQKTQRRWFLDKLVDEIGVGYVVDQGSVSQYYLSQIKPLHNFLTQLQPFLKLKQKQANLVLKIIEQLPSAKESPDKFLEVCTWVDQIAALNDSKTRKTTSETVRAVLD

SEQ ID NO: 199 - protein, 147 aa, synthetic construct
  REGION 1..147: note = Synthetic Polypeptide
KEFLLYLAGFVDGDGSIYARIYPKQSSKFKHHLRLTFHVSQKTQRRWFLDKLVDEIGVGYVYDSGSVSQYYLSEIKPLHNFLTQLQPFLKLKQKQANLVLKIIEQLPSAKESPDKFLEVCTWVDQIAALNDSKTRKTTSETVRAVLD

SEQ ID NO: 200 - protein, 147 aa, synthetic construct
  REGION 1..147: note = Synthetic Polypeptide
KEFLLYLAGFVDGDGSIYARIEPEQTAKFKHMLRLTFRVSQKTQRRWFLDKLVDEIGVGYVNDSGSVSGYYLSQIKPLHNFLTQLQPFLKLKQKQANLVLKIIEQLPSAKESPDKFLEVCTWVDQIAALNDSKTRKTTSETVRAVLD

SEQ ID NO: 201 - protein, 147 aa, synthetic construct; identical to SEQ ID NO: 197
  REGION 1..147: note = Synthetic Polypeptide

SEQ ID NO: 202 - protein, 147 aa, synthetic construct
  REGION 1..147: note = Synthetic Polypeptide
KEFLLYLAGFVDGDGSIYARIEPKQTAKFKHHLCLRFHVSQKTQRRWFLDKLVDEIGVGYVVDHGSVSQYFLSQIKPLHNFLTQLQPFLKLKQKQANLVLKIIEQLPSAKESPDKFLEVCTWVDQIAALNDSKTRKTTSETVRAVLD

SEQ ID NO: 203 - protein, 147 aa, synthetic construct; identical to SEQ ID NO: 193
  REGION 1..147: note = Synthetic Polypeptide

SEQ ID NO: 204 - protein, 147 aa, synthetic construct
  REGION 1..147: note = Synthetic Polypeptide
KEFLLYLAGFVDGDGSIYARIEPRQRVKFKHFLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 205 - protein, 147 aa, synthetic construct
  REGION 1..147: note = Synthetic Polypeptide
KEFLLYLAGFVDGDGSIYARIEPRQGCKFKHVLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 206 - protein, 147 aa, synthetic construct
  REGION 1..147: note = Synthetic Polypeptide
KEFLLYLAGFVDGDGSIYARIEPRQVCKFKHILRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 207 - protein, 147 aa, synthetic construct
  REGION 1..147: note = Synthetic Polypeptide
KEFLLYLAGFVDGDGSIYARIEPRQCFKFKHCLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 208 - protein, 147 aa, synthetic construct
  REGION 1..147: note = Synthetic Polypeptide
KEFLLYLAGFVDGDGSIYARIEPRQRFKFKHCLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 209 - protein, 147 aa, synthetic construct
  REGION 1..147: note = Synthetic Polypeptide
KEFLLYLAGFVDGDGSIYARIEPRQFCKFKHILRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 210 - protein, 147 aa, synthetic construct
  REGION 1..147: note = Synthetic Polypeptide
KEFLLYLAGFVDGDGSIYARIEPRQAFKFKHALRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 211 - protein, 147 aa, synthetic construct
  REGION 1..147: note = Synthetic Polypeptide
KEFLLYLAGFVDGDGSIYARIEPRQTCKFKHILRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 212 - protein, 147 aa, synthetic construct
  REGION 1..147: note = Synthetic Polypeptide
KEFLLYLAGFVDGDGSIYARIEPRQCFKFKHGLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 213 - protein, 147 aa, synthetic construct
  REGION 1..147: note = Synthetic Polypeptide
KEFLLYLAGFVDGDGSIYARIEPRQSSKFKHLLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 214 - protein, 147 aa, synthetic construct
  REGION 1..147: note = Synthetic Polypeptide
KEFLLYLAGFVDGDGSIYARIYLGQHWKFKHYVRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 215 - protein, 147 aa, synthetic construct
  REGION 1..147: note = Synthetic Polypeptide
KEFLLYLAGFVDGDGSIYARIYLGQHYKFKHYIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 216 - protein, 147 aa, synthetic construct
  REGION 1..147: note = Synthetic Polypeptide
KEFLLYLAGFVDGDGSIYARIYNAQYYKFKHYIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 217 - protein, 147 aa, synthetic construct
  REGION 1..147: note = Synthetic Polypeptide
KEFLLYLAGFVDGDGSIYARIERRQNFKFKHHIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 218 - protein, 147 aa, synthetic construct
  REGION 1..147: note = Synthetic Polypeptide
KEFLLYLAGFVDGDGSIYARIERRQCVKFKHYLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 219 - protein, 147 aa, synthetic construct
  REGION 1..147: note = Synthetic Polypeptide
KEFLLYLAGFVDGDGSIYARIEYRQGAKFKHYLRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 220 - protein, 147 aa, synthetic construct
  REGION 1..147: note = Synthetic Polypeptide
KEFLLYLAGFVDGDGSIYARIEPRQSGKFKHYIRLTFHVSQKTQRRWFLDKLVDEIGVGYVTDWGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 221 - protein, 147 aa, synthetic construct
  REGION 1..147: note = Synthetic Polypeptide
KEFLLYLAGFVDGDGSIYARIFKGQHWKFKHHIRLTFNVSQKTQRRWFLDKLVDEIGVGYVSDTGSQSNYYLSEIKPLHNFLTQLQPFLKLKQKQANLVLKIIEQLPSAKESPDKFLEVCTWVDQIAALNDSKTRKTTSETVRAVLD

SEQ ID NO: 222 - protein, 147 aa, synthetic construct
  REGION 1..147: note = Synthetic Polypeptide
KEFLLYLAGFVDGDGSIFARIFKGQHWKFKHHIRLTFNVSQKTQRRWFLDKLVDEIGVGYVNDYGSTSNYYLSEIKPLHNFLTQLQPFLKLKQKQANLVLKIIEQLPSAKESPDKFLEVCTWVDQIAALNDSKTRKTTSETVRAVLD

SEQ ID NO: 223 - protein, 147 aa, synthetic construct
  REGION 1..147: note = Synthetic Polypeptide
KEFLLYLAGFVDGDGSIFARIFKGQHWKFKHHIRLTFNVAQKTQRRWFLDKLVDEIGVGYVTDHGSNSNYYLSEIKPLHNFLTQLQPFLKLKQKQANLVLKIIEQLPSAKESPDKFLEVCTWVDQIAALNDSKTRKTTSETVRAVLD

SEQ ID NO: 224 - protein, 147 aa, synthetic construct
  REGION 1..147: note = Synthetic Polypeptide
KEFLLYLAGFVDGDGSIYARIFKGQHWKFKHHIRLTFNVTQKTQRRWFLDKLVDEIGVGYVSDYGSMSNYYLSEIKPLHNFLTQLQPFLKLKQKQANLVLKIIEQLPSAKESPDKFLEVCTWVDQIAALNDSKTRKTTSETVRAVLD

SEQ ID NO: 225 - protein, 147 aa, synthetic construct
  REGION 1..147: note = Synthetic Polypeptide
KEFLLYLAGFVDGDGSIYARIFKGQHWKFKHHIRLTFAVTQKTQRRWFLDKLVDEIGVGYVTDHGSCSNYYLSEIKPLHNFLTQLQPFLKLKQKQANLVLKIIEQLPSAKESPDKFLEVCTWVDQIAALNDSKTRKTTSETVRAVLD

SEQ ID NO: 226 - protein, 147 aa, synthetic construct
  REGION 1..147: note = Synthetic Polypeptide
KEFLLYLAGFVDGDGSIYARIFKGQHWKFKHHIRLTFNVSQKTQRRWFLDKLVDEIGVGYVGDYGSMSHYYLSEIKPLHNFLTQLQPFLKLKQKQANLVLKIIEQLPSAKESPDKFLEVCTWVDQIAALNDSKTRKTTSETVRAVLD

SEQ ID NO: 227 - protein, 147 aa, synthetic construct
  REGION 1..147: note = Synthetic Polypeptide
KEFLLYLAGFVDGDGSIYARIFKGQHWKFKHHIRLTFNVSQKTQRRWFLDKLVDEIGVGYVTDFGSQSQYYLSEIKPLHNFLTQLQPFLKLKQKQANLVLKIIEQLPSAKESPDKFLEVCTWVDQIAALNDSKTRKTTSETVRAVLD

SEQ ID NO: 228 - protein, 147 aa, synthetic construct
  REGION 1..147: note = Synthetic Polypeptide
KEFLLYLAGFVDGDGSIFARIFKGQHWKFKHHIRLTFNVTQKTQRRWFLDKLVDEIGVGYVSDYGSISNYYLSEIKPLHNFLTQLQPFLKLKQKQANLVLKIIEQLPSAKESPDKFLEVCTWVDQIAALNDSKTRKTTSETVRAVLD

SEQ ID NO: 229 - protein, 147 aa, synthetic construct
  REGION 1..147: note = Synthetic Polypeptide
KEFLLYLAGFVDGDGSIYARIFKGQHWKFKHHIRLTFNVTQKTQRRWFLDKLVDEIGVGYVIDYGSISNYYLSEIKPLHNFLTQLQPFLKLKQKQANLVLKIIEQLPSAKESPDKFLEVCTWVDQIAALNDSKTRKTTSETVRAVLD

SEQ ID NO: 230 - protein, 147 aa, synthetic construct
  REGION 1..147: note = Synthetic Polypeptide
KEFLLYLAGFVDGDGSIYARIFKGQHWKFKHHIRLTFNVTQKTQRRWFLDKLVDEIGVGYVTDYGSSSNYYLSEIKPLHNFLTQLQPFLKLKQKQANLVLKIIEQLPSAKESPDKFLEVCTWVDQIAALNDSKTRKTTSETVRAVLD

SEQ ID NO: 231 - protein, 147 aa, synthetic construct
  REGION 1..147: note = Synthetic Polypeptide
KEFLLYLAGFVDGDGSIFARIFKGQHWKFKHHIRLTFNVSQKTQRRWFLDKLVDEIGVGYVTDGGSSSNYYLSEIKPLHNFLTQLQPFLKLKQKQANLVLKIIEQLPSAKESPDKFLEVCTWVDQIAALNDSKTRKTTSETVRAVLD

SEQ ID NO: 232 - protein, 147 aa, synthetic construct
  REGION 1..147: note = Synthetic Polypeptide
KEFLLYLAGFVDGDGSIFARIFKGQHWKFKHHIRLTFNVSQKTQRRWFLDKLVDEIGVGYVYDHGSQSNYYLSEIKPLHNFLTQLQPFLKLKQKQANLVLKIIEQLPSAKESPDKFLEVCTWVDQIAALNDSKTRKTTSETVRAVLD

SEQ ID NO: 233 - protein, 147 aa, synthetic construct
  REGION 1..147: note = Synthetic Polypeptide
KEFLLYLAGFVDGDGSIYARIFKGQHWKFKHHIRLTFNVSQKTQRRWFLDKLVDEIGVGYVCDNGSNSNYYLSEIKPLHNFLTQLQPFLKLKQKQANLVLKIIEQLPSAKESPDKFLEVCTWVDQIAALNDSKTRKTTSETVRAVLD

SEQ ID NO: 234 - protein, 147 aa, synthetic construct
  REGION 1..147: note = Synthetic Polypeptide
KEFLLYLAGFVDGDGSIFARIFKGQHWKFKHHIRLTFNVSQKTQRRWFLDKLVDEIGVGYVADYGSTSNYYLSEIKPLHNFLTQLQPFLKLKQKQANLVLKIIEQLPSAKESPDKFLEVCTWVDQIAALNDSKTRKTTSETVRAVLD

SEQ ID NO: 235 - protein, 147 aa, synthetic construct
  REGION 1..147: note = Synthetic Polypeptide
KEFLLYLAGFVDGDGSIYARIFKGQHWKFKHHIRLTFCVSQKTQRRWFLDKLVDEIGVGYVADYGSMSNYYLSEIKPLHNFLTQLQPFLKLKQKQANLVLKIIEQLPSAKESPDKFLEVCTWVDQIAALNDSKTRKTTSETVRAVLD

SEQ ID NO: 236 - protein, 147 aa, synthetic construct
  REGION 1..147: note = Synthetic Polypeptide
KEFLLYLAGFVDGDGSIMARIFKGQHWKFKHYIRLTLCVRQKTQRRWFLDKLVDEIGVGYVCDSGSMSAYYLSEIKPLHNFLTQLQPFLKLKQKQANLVLKIIEQLPSAKESPDKFLEVCTWVDQIAALNDSKTRKTTSETVRAVLD

SEQ ID NO: 237 - protein, 147 aa, synthetic construct; identical to SEQ ID NO: 231
  REGION 1..147: note = Synthetic Polypeptide

SEQ ID NO: 238 - protein, 147 aa, synthetic construct; identical to SEQ ID NO: 192
  REGION 1..147: note = Synthetic Polypeptide

SEQ ID NO: 239 - protein, 147 aa, synthetic construct; identical to SEQ ID NO: 192
  REGION 1..147: note = Synthetic Polypeptide

SEQ ID NO: 240 - protein, 147 aa, synthetic construct; identical to SEQ ID NO: 192
  REGION 1..147: note = Synthetic Polypeptide

SEQ ID NO: 241 - protein, 147 aa, synthetic construct; identical to SEQ ID NO: 192
  REGION 1..147: note = Synthetic Polypeptide

SEQ ID NO: 242 - protein, 147 aa, synthetic construct; identical to SEQ ID NO: 192
  REGION 1..147: note = Synthetic Polypeptide

SEQ ID NO: 243 - protein, 147 aa, synthetic construct; identical to SEQ ID NO: 192
  REGION 1..147: note = Synthetic Polypeptide

SEQ ID NO: 244 - protein, 147 aa, synthetic construct; identical to SEQ ID NO: 192
  REGION 1..147: note = Synthetic Polypeptide

SEQ ID NO: 245 - protein, 147 aa, synthetic construct; identical to SEQ ID NO: 192
  REGION 1..147: note = Synthetic Polypeptide

SEQ ID NO: 246 - protein, 147 aa, synthetic construct; identical to SEQ ID NO: 192
  REGION 1..147: note = Synthetic Polypeptide

SEQ ID NO: 247 - protein, 147 aa, synthetic construct; identical to SEQ ID NO: 192
  REGION 1..147: note = Synthetic Polypeptide

SEQ ID NO: 248 - protein, 147 aa, synthetic construct; identical to SEQ ID NO: 192
  REGION 1..147: note = Synthetic Polypeptide

SEQ ID NO: 249 - protein, 147 aa, synthetic construct
  REGION 1..147: note = Synthetic Polypeptide
KEFLLYLAGFVDGDGSIYARIFKGQHWKFKHYIRLTFCVSQKTQRRWFLDKLVDEIGVGYVNDSGSCSNYYLSEIKPLHNFLTQLQPFLKLKQKQANLVLKIIEQLPSAKESPDKFLEVCTWVDQIAALNDSKTRKTTSETVRAVLD

SEQ ID NO: 250 - protein, 147 aa, synthetic construct; identical to SEQ ID NO: 249
  REGION 1..147: note = Synthetic Polypeptide

SEQ ID NO: 251 - protein, 147 aa, synthetic construct
  REGION 1..147: note = Synthetic Polypeptide
KEFLLYLAGFVDGDGSIYARIFKGQHWKFKHHIRLTFNVSQKTQRRWFLDKLVDEIGVGYVCDSGSQSNYYLSEIKPLHNFLTQLQPFLKLKQKQANLVLKIIEQLPSAKESPDKFLEVCTWVDQIAALNDSKTRKTTSETVRAVLD

SEQ ID NO: 252 - protein, 147 aa, synthetic construct
  REGION 1..147: note = Synthetic Polypeptide
KEFLLYLAGFVDGDGSIFARIFKGQHWKFKHHIRLTFCVSQKTQRRWFLDKLVDEIGVGYVVDNGSMSNYYLSEIKPLHNFLTQLQPFLKLKQKQANLVLKIIEQLPSAKESPDKFLEVCTWVDQIAALNDSKTRKTTSETVRAVLD

SEQ ID NO: 253 - protein, 147 aa, synthetic construct
  REGION 1..147: note = Synthetic Polypeptide
KEFLLYLAGFVDGDGSIYARIFKGQHWKFKHHIRLTFCVTQKTQRRWFLDKLVDEIGVGYVHDYGSCSNYYLSEIKPLHNFLTQLQPFLKLKQKQANLVLKIIEQLPSAKESPDKFLEVCTWVDQIAALNDSKTRKTTSETVRAVLD

SEQ ID NO: 254 - protein, 147 aa, synthetic construct; identical to SEQ ID NO: 194
  REGION 1..147: note = Synthetic Polypeptide

SEQ ID NO: 255 - protein, 147 aa, synthetic construct; identical to SEQ ID NO: 194
  REGION 1..147: note = Synthetic Polypeptide

SEQ ID NO: 256 - protein, 147 aa, synthetic construct; identical to SEQ ID NO: 194
  REGION 1..147: note = Synthetic Polypeptide

SEQ ID NO: 257 - protein, 147 aa, synthetic construct; identical to SEQ ID NO: 194
  REGION 1..147: note = Synthetic Polypeptide

SEQ ID NO: 258 - protein, 147 aa, synthetic construct; identical to SEQ ID NO: 197
  REGION 1..147: note = Synthetic Polypeptide

SEQ ID NO: 259 - protein, 147 aa, synthetic construct; identical to SEQ ID NO: 197
  REGION 1..147: note = Synthetic Polypeptide

SEQ ID NO: 260 - protein, 147 aa, synthetic construct; identical to SEQ ID NO: 197
  REGION 1..147: note = Synthetic Polypeptide

SEQ ID NO: 261 - protein, 147 aa, synthetic construct; identical to SEQ ID NO: 197
  REGION 1..147: note = Synthetic Polypeptide

SEQ ID NO: 262 - protein, 147 aa, synthetic construct; identical to SEQ ID NO: 197
  REGION 1..147: note = Synthetic Polypeptide

SEQ ID NO: 263 - protein, 147 aa, synthetic construct; identical to SEQ ID NO: 197
  REGION 1..147: note = Synthetic Polypeptide

SEQ ID NO: 264 - protein, 147 aa, synthetic construct; identical to SEQ ID NO: 197
  REGION 1..147: note = Synthetic Polypeptide

SEQ ID NO: 265 - protein, 147 aa, synthetic construct; identical to SEQ ID NO: 197
  REGION 1..147: note = Synthetic Polypeptide

SEQ ID NO: 266 - protein, 147 aa, synthetic construct; identical to SEQ ID NO: 197
  REGION 1..147: note = Synthetic Polypeptide

SEQ ID NO: 267 - protein, 147 aa, synthetic construct; identical to SEQ ID NO: 197
  REGION 1..147: note = Synthetic Polypeptide

SEQ ID NO: 268 - protein, 147 aa, synthetic construct; identical to SEQ ID NO: 197
  REGION 1..147: note = Synthetic Polypeptide

SEQ ID NO: 269 - protein, 147 aa, synthetic construct; identical to SEQ ID NO: 197
  REGION 1..147: note = Synthetic Polypeptide

SEQ ID NO: 270 - protein, 147 aa, synthetic construct; identical to SEQ ID NO: 197
  REGION 1..147: note = Synthetic Polypeptide

SEQ ID NO: 271 - protein, 147 aa, synthetic construct; identical to SEQ ID NO: 193
  REGION 1..147: note = Synthetic Polypeptide

SEQ ID NO: 272 - protein, 147 aa, synthetic construct
  REGION 1..147: note = Synthetic Polypeptide
KEFLLYLAGFVDGDGSIYACIEPKQTSKFKHRLCLRFHVSQKTQRRWFLDKLVDEIGVGYVHDNGSVSQYYLSEIKPLHNFLTQLQPFLKLKQKQANLVLKIIEQLPSAKESPDKFLEVCTWVDQIAALNDSKTRKTTSETVRAVLD

SEQ ID NO: 273 - protein, 147 aa, synthetic construct
  REGION 1..147: note = Synthetic Polypeptide
KEFLLYLAGFVDGDGSIYARIYPGQHYKFKHYLRLTFHVSQKTQRRWFLDKLVDEIGVGYVTDAGSVSQYFLSQIKPLHNFLTQLQPFLKLKQKQANLVLKIIEQLPSAKESPDKFLEVCTWVDQIAALNDSKTRKTTSETVRAVLD

SEQ ID NO: 274 - protein, 147 aa, synthetic construct
  REGION 1..147: note = Synthetic Polypeptide
KEFLLYLAGFVDGDGSIYARIEPRQTAKFKHYLRLTFHVSQKTQRRWFLDKLVDEIGVGYVIDAGSVSGYYLSEIKPLHNFLTQLQPFLKLKQKQANLVLKIIEQLPSAKESPDKFLEVCTWVDQIAALNDSKTRKTTSETVRAVLD

SEQ ID NO: 275 - protein, 147 aa, synthetic construct
  REGION 1..147: note = Synthetic Polypeptide
KEFLLYLAGFVDGDGSIYACIEPKQTSKFKHRLCLRFHVSQKTQRRWFLDKLVDEIGVGYVHDNGSVSQYYLSQIKPLHNFLTQLQPFLKLKQKQANLVLKIIEQLPSAKESPDKFLEVCTWVDQIAALNDSKTRKTTSETVRAVLD

SEQ ID NO: 276 - protein, 147 aa, synthetic construct
  REGION 1..147: note = Synthetic Polypeptide
KEFLLYLAGFVDGDGSIYARIEPRQTAKFKHQLRLTFHVSQKTQRRWFLDKLVDEIGVGYVTDWGSVSGYYLSQIKPLHNFLTQLQPFLKLKQKQANLVLKIIEQLPSAKESPDKFLEVCTWVDQIAALNDSKTRKTTSETVRAVLD

SEQ ID NO: 277 - protein, 147 aa, synthetic construct
  REGION 1..147: note = Synthetic Polypeptide
KEFLLYLAGFVDGDGSIYARIEPRQTVKFKHMLRLTFHVSQKTQRRWFLDKLVDEIGVGYVHDNGSVSQYYLSQIKPLHNFLTQLQPFLKLKQKQANLVLKIIEQLPSAKESPDKFLEVCTWVDQIAALNDSKTRKTTSETVRAVLD

SEQ ID NO: 278 - DNA, 6214 bp, synthetic construct
  misc_feature 1..6214: note = Synthetic Polynucleotide
  source 1..6214: mol_type = other DNA
cagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgttgatgcggttttggcagtacaccaatgggcgtggatagcggtttgactcacggggatttccaagtctccaccccattgacgtcaatgggagtttgtttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcaccgaagaagaagcgcaaggtgcatatgaatacaaaatataataaagagttcttactctacttagcagggtttgtagacggtgacggttccatctttgcccgtatctttaagggtcaacattggaagttcaagcactatattcgtttgaccttcacggtggctcagaagacacagcgccgttggttcctcgacaagctggtggacgagatcggtgtgggttacgtgactgactggggcagcgtgtcccattactatctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctgggcctcgatcattcctgagcaaggttataagttcaagcacaggctgcgtctctctttcactgtcgctcagaagacacagcgccgttggttcctcgacaagctggtggacgagatcggtgtgggttacgtggttgaccagggcagcgtctccgagtacaggctgtccgagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtaccagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgggcgaccaaaggtcgcccgacgcccgggcaaagcccgggcgtcgggcgacctttggtcgcccggcctcagtgagcgagcgagcgcgcagagagggagtggccaaccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 279 - DNA, 6214 bp, synthetic construct
  misc_feature 1..6214: note = Synthetic Polynucleotide
  source 1..6214: mol_type = other DNA
cagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgttgatgcggttttggcagtacaccaatgggcgtggatagcggtttgactcacggggatttccaagtctccaccccattgacgtcaatgggagtttgtttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcaccgaagaagaagcgcaaggtgcatatgaatacaaaatataataaagagttcttactctacttagcagggtttgtagacggtgacggttccatctatgcccgtatctttaagggtcaacattggaagttcaagcactatattcgtttgaccttctgtgtgcggcagaagacacagcgccgttggttcctcgacaagctggtggacgagatcggtgtgggttacgtgactgactctggcagcgtttccgcttactatctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctgggcctcgatcattcctgagcaaggttataagttcaagcacaggctgcgtctctctttcactgtcgctcagaagacacagcgccgttggttcctcgacaagctggtggacgagatcggtgtgggttacgtggttgaccagggcagcgtctccgagtacaggctgtccgagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtaccagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgggcgaccaaaggtcgcccgacgcccgggcaaagcccgggcgtcgggcgacctttggtcgcccggcctcagtgagcgagcgagcgcgcagagagggagtggccaaccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 280 - DNA, 5888 bp, synthetic construct
  misc_feature 1..5888: note = Synthetic Polynucleotide
  source 1..5888: mol_type = other DNA
cagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcaccgaagaagaagcgcaaggtgcatatgaatacaaaatataataaagagttcttactctacttagcagggtttgtagacggtgacggttccatctttgcccgtatctttaagggtcaacattggaagttcaagcactatattcgtttgaccttcacggtggctcagaagacacagcgccgttggttcctcgacaagctggtggacgagatcggtgtgggttacgtgactgactggggcagcgtgtcccattactatctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctgggcctcgatcattcctgagcaaggttataagttcaagcacaggctgcgtctctctttcactgtcgctcagaagacacagcgccgttggttcctcgacaagctggtggacgagatcggtgtgggttacgtggttgaccagggcagcgtctccgagtacaggctgtccgagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 281 - DNA, 5888 bp, synthetic construct
  misc_feature 1..5888: note = Synthetic Polynucleotide
  source 1..5888: mol_type = other DNA
cagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcaccgaagaagaagcgcaaggtgcatatgaatacaaaatataataaagagttcttactctacttagcagggtttgtagacggtgacggttccatctatgcccgtatctttaagggtcaacattggaagttcaagcactatattcgtttgaccttctgtgtgcggcagaagacacagcgccgttggttcctcgacaagctggtggacgagatcggtgtgggttacgtgactgactctggcagcgtttccgcttactatctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctgggcctcgatcattcctgagcaaggttataagttcaagcacaggctgcgtctctctttcactgtcgctcagaagacacagcgccgttggttcctcgacaagctggtggacgagatcggtgtgggttacgtggttgaccagggcagcgtctccgagtacaggctgtccgagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

(19) United States
(12) Patent Application Publication
(10) Pub. No.: US 2025/0121039 A1
(43) Pub. Date: Apr. 17, 2025

(54) TREATMENT OF RETINITIS PIGMENTOSA USING ENGINEERED MEGANUCLEASES

(71) Applicant: Precision BioSciences, Inc., Durham, NC (US)

(72) Inventors: Victor Bartsevich, Durham, NC (US); Derek Jantz, Durham, NC (US); J. Jefferson Smith, Morrisville, NC (US); Michael G. Nicholson, Chapel Hill, NC (US)

(73) Assignee: Precision BioSciences, Inc., Durham, NC (US)

(21) Appl. No.: 18/987,584

(22) Filed: Dec. 19, 2024

Related U.S. Application Data
(63) Continuation of application No. 18/351,633, filed on Jul. 13, 2023.
Application No. 18/351,633 is a continuation of application No. 17/585,527, filed on Jan. 26, 2022, now abandoned.
Application No. 17/585,527 is a continuation of application No. 16/932,653, filed on Jul. 17, 2020, now abandoned.
Application No. 16/932,653 is a continuation of application No. 16/791,687, filed on Feb. 14, 2020, now Pat. No. 10,758,595.
Application No. 16/791,687 is a continuation of application No. 15/758,366, filed on Mar. 8, 2018, now Pat. No. 10,603,363.
(60) Provisional application No. 62/215,460, filed on Sep. 8, 2015.

(51) Int. Cl. A61K 38/46; A61K 48/00; A61P 27/00; C12N 9/22; C12N 15/52; C12N 15/861; C12N 15/90

(52) U.S. Cl. CPC A61K 38/465; A61K 48/0058; C12N 9/22; C12N 15/52; C12N 15/861; C12N 15/907; A61P 27/00; C12N 2750/14143

(57) ABSTRACT

Disclosed are recombinant meganucleases engineered to recognize and cleave recognition sequences present in a mutant RHO P23H allele. The invention further relates to the use of such recombinant meganucleases in methods for treating retinitis pigmentosa, wherein the mutant RHO P23H allele is preferentially targeted, cleaved, and inactivated.

### CROSS REFERENCE TO RELATED APPLICATIONS

This application is a continuation of U.S. application Ser. No. 18/351,633, filed Jul. 13, 2023, which is a continuation of U.S. application Ser. No. 17/585,527, filed Jan. 26, 2022, now abandoned, which is a continuation of U.S. application Ser. No. 16/932,653, filed Jul. 17, 2020, now abandoned, which is a continuation of U.S. application Ser. No. 16/791,687, filed Feb. 14, 2020, now U.S. Pat. No. 10,758,595, which is a continuation of U.S. application Ser. No. 15/758,366, filed Mar. 8, 2018, now U.S. Pat. No. 10,603,363, which is a National Stage Entry of PCT/US2016/050809, filed Sep. 8, 2016, which claims the benefit of priority under 35 U.S.C. § 119 (e) to U.S. Provisional Application No. 62/215,460, filed Sep. 8, 2015, the contents of which are incorporated herein in their entirety by reference.

### FIELD OF THE INVENTION

The invention relates to the field of molecular biology and recombinant nucleic acid technology. In particular, the invention relates to recombinant meganucleases engineered to recognize and cleave a recognition sequence found in a human rhodopsin gene allele. The invention further relates to the use of such recombinant meganucleases in methods for treating retinitis pigmentosa.

### REFERENCE TO AN ELECTRONIC SEQUENCE LISTING

The instant application contains an electronic sequence listing which has been submitted in xml (ST.26) format via USPTO Patent Center, hereby incorporated by reference in its entirety. Said xml copy, created on Jul. 11, 2023, is named “P89339 1440US.C4 (1089.1) Seq List ST.26.xml” and is 461,767 bytes in size.

### BACKGROUND OF THE INVENTION

Retinitis pigmentosa (RP) is an inherited degenerative eye disease that causes severe vision impairment due to the progressive degeneration of photoreceptor cells in the retina. RP is characterized by an initial decline in rod photoreceptor cells, resulting in compromised peripheral and dim light vision. Progressive rod degeneration is followed by abnormalities in the retinal pigment epithelium and deterioration of cone photoreceptor cells. As the disease advances, patients experience nyctalopia, progressive tunnel vision, and eventual blindness. RP affects approximately 1 in 3000 people and can occur alone or together with other systemic disorders. Currently, RP has no effective treatment.

The genetic causes of RP have been identified as autosomal dominant, autosomal recessive, X-linked, or maternally acquired. The autosomal dominant form of RP represents 30-40% of cases (Ma et al. (2105), Scientific Reports. 18 (5:9236): 1-6), and has been associated with mutations in genes expressed in rod photoreceptor cells and the retinal pigment epithelium. The human rhodopsin gene (RHO) was the first gene shown to contribute to the pathogenesis of autosomal dominant RP and remains the most common gene associated with this form of the disease (McWilliam et al. (1989) Genomics. 5:619-622; Dryja et al. (1990) Nature. 343:364-366; Farrar et al. (1990) EMBO Journal. 21:857-864). Indeed, RHO mutations are associated with 30-40% of autosomal dominant RP cases worldwide, and are observed in approximately 26.5% of cases in the United States (Illing et al. (2002) Journal of Bio. Chem. 277 (37): 34150-34160).

Rhodopsin is an essential photopigment expressed in retinal rod photoreceptor cells that is responsible for the conversion of light stimuli into electrical signals in the first step of phototransduction. Rhodopsin is expressed as a light-sensitive G-protein-coupled receptor that consists of an opsin protein moiety bound to an 11-cis-retinal chromophore, and represents the main component of the disk membranes of rod photoreceptor cell outer segments.

The first RHO mutation shown to contribute to autosomal dominant RP was a C to A mutation at position 68 of the RHO gene coding sequence, which confers a proline to histidine substitution at position 23 (P23H) of the encoded protein. This mutation is referred to herein as the “RHO P23H mutation,” and a RHO allele comprising the mutation is referred to herein as a “mutant RHO P23H allele.” The RHO P23H mutation is the most frequently reported RHO mutation in autosomal dominant RP cases in North America (Mao et al. (2011) Human Gene Therapy. 22:567-575), and patients having a single mutant RHO P23H allele can develop RP despite the presence of a functional wild-type RHO allele.

Rhodopsin proteins that comprise the P23H substitution fold improperly, accumulate in the endoplasmic reticulum of rod photoreceptor cells, and do not reconstitute with the 11-cis-retinal chromophore. In many cases of autosomal dominant RP, misfolded P23H rhodopsin contributes to rod photoreceptor cell degeneration and death. Accumulated P23H rhodopsin undergoes proteasomal and lysosomal degradation and has been shown to stimulate the ER-associated unfolded protein response, which can induce ER stress and cellular apoptosis (Lin et al. (2007), Science. 318:944-949; Gorbatyuk et al. (2010) PNAS U.S.A. 107 (13): 5961-5966). Misfolding of P23H rhodopsin may also contribute to cell death by interfering with the transport or function of wild-type rhodopsin (Illing et al., 2002, Lin et al., 2007). Furthermore, P23H rhodopsin has been shown to exhibit delayed dephosphorylation, and cell death may result from abnormal cytosolic Ca²⁺ levels (Saito et al. (2008) Clin. Opthamol. 2:821-828).

Multiple strategies have been pursued to treat autosomal dominant RP, including nutritional therapies, pharmaceuticals, and gene therapy. Gene therapy approaches have adopted either an indirect or a direct strategy for treating autosomal dominant RP. Indirect approaches have aimed to promote the survival of rod photoreceptor cells without directly affecting the expression of pathogenic mutant proteins For example, gene therapy has been used to introduce neurotrophic factors, such as GDNF, and anti-apoptotic proteins, such as XIAP, in retinal cells in order to inhibit apoptosis in rod photoreceptor cells.

By contrast, direct approaches in gene therapy have sought to modulate the levels of proteins that directly contribute to the pathogenesis of autosomal dominant RP. In the context of RHO-associated autosomal dominant RP, one strategy has been to enhance the proteasomal degradation of P23H rhodopsin, though no significant success has been made in animal models. Another strategy has utilized targeted RNA-based therapy to silence a mutant RHO allele while maintaining expression of the functional wild-type allele. Such approaches have used ribozymes and RNA interference (RNAi) to target specific mRNA transcripts produced by a mutant RHO P23H transgene in rats.

Further strategies have pursued a “suppression and replacement” approach by non-specifically silencing both the wild-type RHO allele and the mutant RHO allele, while concurrently delivering a replacement copy of wild-type RHO to express the wild-type protein. For example, O'Reilly et al. utilized adeno-associated virus (AAV) vectors to deliver and express short hairpin RNAs designed to target and suppress both the wild-type and mutant RHO alleles in heterozygous Pro23His^{+/-} mice, while also delivering and expressing a RHO replacement gene (O'Reilly et al. (2007) Amer. J. of Human Genetics. 81:127-135). Palfi et al. similarly demonstrated the use of AAV vectors to deliver a RHO replacement gene to Rho./− knockout mice (Palfi et al. (2010) Human Gene Therapy. 21:311-323). However, in such approaches, toxicity and off-target effects may be induced if RHO replacement levels are too high. Furthermore, off-target effects of RNAi approaches are a known complication, and it has been shown that siRNAs greater than 21 base pairs in length can induce retinal degeneration in animal models (Kleinman et al. (2012) Mol. Ther. 20 (1): 101-108).

The use of engineered nucleases for cleaving DNA targets in the human RHO gene was previously disclosed in U.S. Patent Publication No. US 2012/0204282 A1 by Zhang (“the Zhang application”). The Zhang application disclosed several approaches for targeting and modulating the expression of mutant RHO alleles. Specifically, the Zhang application discussed the use of engineered DNA binding domains, such as zinc finger proteins (ZFP) and TAL effector (TALE) proteins, as repressors of RHO gene expression. The Zhang application also described fusion proteins comprising a ZFP or TALE binding domain operably linked to a regulatory or functional domain. The functional domain could be a transcriptional repressor domain that downregulates RHO gene expression. Alternatively, the functional domain could be a transcriptional activation domain. Further, the functional domain could comprise a nuclease domain. When linked to a nuclease domain, the resulting fusion proteins include zinc finger nucleases (ZFNs) and TALE-nucleases (TALENs).

In addition to ZFNs and TALENs, the Zhang application discusses the use of meganucleases for targeting and inhibiting the expression of wild-type and/or mutant RHO alleles. The Zhang application describes the use of such meganucleases for disrupting RHO gene expression via non-homologous end joining (NHEJ) at the recognition sequence, and for introducing a replacement wild-type RHO gene sequence to express the wild-type rhodopsin protein. However, the recognition sequences in the RHO gene that are identified by the Zhang application are limited to three pairs of ZFN target sites found in the wild-type RHO gene (see, Zhang application at Table 2).

The use of engineered meganucleases for cleaving DNA targets in the RHO gene was also disclosed in U.S. Patent Publication No. US 2013/0183282 by Lemaire and Arnould (“the Lemaire application”). The Lemaire application disclosed meganucleases designed to target various regions of the RHO gene for use in one of three gene therapy strategies. The first strategy is gene correction, wherein the engineered meganucleases are specific for a recognition sequence in the vicinity of a specified mutation, induce a double-strand break at that site, and rely on homologous recombination of a corresponding non-mutant allelic sequence into the genome. The second strategy disclosed in the Lemaire application is exon knock-in, wherein a functional protein is reconstituted by using a meganuclease to introduce a synthetic wild-type coding sequence into the genome while preventing the expression of the pathologic mutation. The third strategy disclosed in the Lemaire application is gene inactivation by mutagenesis, which relies a meganuclease to induce a double-strand break at a target recognition sequence in the genome, and NHEJ at the cleavage site to induce a mutation.

Accordingly, there is still a need in the art for methods that can preferentially target and inactivate the RHO P23H allele for treatment of RP.

### SUMMARY OF THE INVENTION

The present invention provides recombinant meganucleases that are engineered to recognize and cleave P23H recognition sequences. The present invention further provides methods comprising the delivery of such a recombinant meganuclease, or genes encoding such a recombinant meganuclease, to the cells of a patient having RP, such that the recombinant meganuclease (or encoded recombinant meganuclease) preferentially targets and cleaves a P23H recognition sequence present on the mutant RHO P23H allele. NHEJ occurs at the cleavage site, resulting in mutagenesis and disruption of the mutant RHO P23H allele, while the functional wild-type RHO allele remains intact to express wild-type rhodopsin in rod photoreceptor cells of the retina. Preferential inactivation of the mutant RHO P23H allele, and disruption of P23H rhodopsin expression, is expected to delay, prevent, or reverse the progression of RP in patients.

Thus, in some embodiments, the present invention provides recombinant meganucleases that are engineered to recognize and cleave a P23H recognition sequence, which is present in the mutant RHO P23H allele but not in the wild-type RHO allele. The present invention further provides the use of such a recombinant meganuclease in a method for treating RP, preferably autosomal dominant RP, wherein the mutant RHO P23H allele is preferentially targeted and cleaved. In this manner, expression of P23H rhodopsin is suppressed due to NHEJ at the meganuclease cleavage site, while the functional wild-type RHO allele remains intact to express wild-type rhodopsin in rod photoreceptor cells of the retina.

Thus, the invention includes the use of site-specific, rare-cutting, homing endonucleases (also called “meganucleases”) that are engineered to recognize specific DNA sequences in a locus of interest. Homing endonucleases are a group of naturally-occurring nucleases which recognize 15-40 base pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38:49-95). Homing endonucleases are commonly grouped into four families: the LAGLIDADG (SEQ ID NO:96) family, the GIY-YIG family, the His-Cys box family and the HNH family. These families are characterized by structural motifs, which affect catalytic activity and recognition sequence. For instance, members of the LAGLIDADG (SEQ ID NO: 96) family are characterized by having either one or two copies of the conserved LAGLIDADG (SEQ ID NO:96) motif (Chevalier et al. (2001), Nucleic Acids Res. 29 (18): 3757-3774). The LAGLIDADG (SEQ ID NO:96) homing endonucleases with a single copy of the LAGLIDADG (SEQ ID NO:96) motif form homodimers, whereas members with two copies of the LAGLIDADG (SEQ ID NO:96) motif are found as monomers.

Methods for producing engineered, site-specific recombinant meganucleases are known in the art. I-CreI (SEQ ID NO:95) is a member of the LAGLIDADG (SEQ ID NO:96) family of homing endonucleases which recognizes and cuts a 22 base pair recognition sequence in the chloroplast chromosome of the algae Chlamydomonas reinhardtii. Genetic selection techniques have been used to modify the wild-type I-CreI cleavage site preference (Sussman et al. (2004), J. Mol. Biol. 342:31-41; Chames et al. (2005), Nucleic Acids Res. 33: €178; Seligman et al. (2002), Nucleic Acids Res. 30:3870-9, Arnould et al. (2006), J. Mol. Biol. 355:443-58). More recently, a method of rationally-designing mono-LAGLIDADG (SEQ ID NO: 96) homing endonucleases was described which is capable of comprehensively redesigning I-CreI and other homing endonucleases to target widely-divergent DNA sites, including sites in mammalian, yeast, plant, bacterial, and viral genomes (WO 2007/047859).

As first described in WO 2009/059195, I-CreI and its engineered derivatives are normally dimeric but can be fused into a single polypeptide using a short peptide linker that joins the C-terminus of a first subunit to the N-terminus of a second subunit (see also Li, et al. (2009) Nucleic Acids Res. 37:1650-62; Grizot, et al. (2009) Nucleic Acids Res. 37:5405-19.) Thus, a functional “single-chain” meganuclease can be expressed from a single transcript. Such engineered meganucleases exhibit an extremely low frequency of off-target cutting. By delivering a gene encoding a single-chain meganuclease to retinal cells, and preferably to rod photoreceptor cells, it is possible to specifically and preferentially target, cleave, and inactivate the mutant RHO P23H allele, thus suppressing expression of P23H rhodopsin.

Thus, in one aspect, the invention provides a recombinant meganuclease that recognizes and cleaves a recognition sequence comprising the mutation which encodes P23H substitution in the P23H mutant allele. In some embodiments, the recognition sequence is selected from the SEQ ID NOs: 1-4 (i.e., the P23H recognition sequences). The recombinant meganuclease comprises a first subunit and a second subunit, wherein the first subunit recognizes a first recognition half-site of one of the P23H recognition sequences and comprises an (a) an amino acid sequence having at least 80% sequence identity to residues 198-344 of any one of SEQ ID NOs: 6-69 or residues 7-153 of any one of SEQ ID NOs: 70-93; and (b) a first hypervariable (HVR1) region which determines specificity for the first recognition half-site. The recombinant meganuclease further comprises a second subunit that recognizes a second half-site of one of the P23H recognition sequences and comprises: (a) an amino acid sequence having at least 80% sequence identity to residues 7-153 of any one of SEQ ID NOs: 6-69 or residues 198-344 of any one of SEQ ID NOs: 70-93; and (b) a second hypervariable (HVR2) region which determines specificity for the second recognition half-site. In some embodiments, the P23H recognition sequence is SEQ ID NO:1 and the recombinant meganuclease comprises a first subunit and a second subunit, wherein the first subunit recognizes a first recognition half-site of one of SEQ ID NO: 1 and comprises (a) an amino acid sequence having at least 80% sequence identity to residues 198-344 of any one of SEQ ID NOs: 6-69 or residues 7-153 of any one of SEQ ID NOs: 70-93; and (b) a first hypervariable (HVR1) region which determines specificity for the first recognition half-site. The recombinant meganuclease further comprises a second subunit that recognizes a second half-site of SEQ ID NO: 1 and comprises: (a) an amino acid sequence having at least 80% sequence identity to residues 7-153 of any one of SEQ ID NOs: 6-69 or residues 198-344 of any one of SEQ ID NOs: 70-93; and (b) a second hypervariable (HVR2) region which determines specificity for the second recognition half-site.

In some embodiments, the P23H recognition sequence is SEQ ID NO: 1 and the first subunit comprises an amino acid sequence having at least 85%, 90%, or 95% sequence identity to residues 198-344 of any one of SEQ ID NOs: 6-69 or residues 7-153 of any one of SEQ ID NOs: 70-93, and the second subunit comprises an amino acid sequence having at least 85%, 90%, or 95% sequence identity to residues 7-153 of any one of SEQ ID NOs: 6-69 or residues 198-344 of any one of SEQ ID NOs: 70-93.

In other embodiments, the P23H recognition sequence is SEQ ID NO: 1 and the HVRI region comprises W or Y at a position corresponding to: (a) position 215 of any one of SEQ ID NOs: 6-69; or (b) position 24 of any one of SEQ ID NOs: 70-93. In other embodiments, the P23H recognition sequence is SEQ ID NO: 1 and the HVRI region comprises I at a position corresponding to: (a) position 219 of any one of SEQ ID NOs: 6-69; or (b) position 28 of any one of SEQ ID NOs: 70-93. In other embodiments, the P23H recognition sequence is SEQ ID NO:1 and the HVRI region comprises V at a position corresponding to: (a) position 259 of any one of SEQ ID NOs: 6-69; or (b) position 68 of any one of SEQ ID NOs: 70-93. In other embodiments, the P23H recognition sequence is SEQ ID NO: 1 and the HVRI region comprises one or more of W or Y, I, and V at positions corresponding to (a) positions 215, 219, and 259, respectively, of any one of SEQ ID NOs: 6-69; or (b) positions 24, 28, and 68, respectively, of any one of SEQ ID NOs: 70-93.

In other embodiments, the P23H recognition sequence is SEQ ID NO: 1 and the HVR2 region comprises Y or M at a position corresponding to (a) position 24 of any one of SEQ ID NOs: 6-69; or (b) position 215 of any one of SEQ ID NOs: 70-93. In other embodiments, the P23H recognition sequence is SEQ ID NO:1 and the HVR2 region comprises F at a position corresponding to (a) position 28 of any one of SEQ ID NOs: 6-69; or (b) position 219 of any one of SEQ ID NOs: 70-93. In other embodiments, the P23H recognition sequence is SEQ ID NO:1 and the HVR2 region comprises H at a position corresponding to (a) position 44 of any one of SEQ ID NOs: 6-69; or (b) position 235 of any one of SEQ ID NOs: 70-93. In other embodiments, the P23H recognition sequence is SEQ ID NO: 1 and the HVR2 region comprises S at a position corresponding to (a) position 46 of any one of SEQ ID NOs: 6-69; or (b) position 237 of any one of SEQ ID NOs: 70-93. In other embodiments, the P23H recognition sequence is SEQ ID NO:1 and the HVR2 region comprises W at a position corresponding to (a) position 70 of any one of SEQ ID NOs: 6-69; or (b) position 261 of any one of SEQ ID NOs: 70-93. In other embodiments, the P23H recognition sequence is SEQ ID NO: 1 and the HVR2 region comprises one or more of Y or M, F, H, S, and W at positions corresponding to (a) positions 24, 28, 44, 46, and 70, respectively, of any one of SEQ ID NOs: 6-69; or (b) positions 215, 219, 235, 237, and 261, respectively, of any one of SEQ ID NOs: 70-93.

In some embodiments, the recombinant meganuclease is a single-chain meganuclease comprising a linker, wherein the linker covalently joins the first subunit and the second subunit.

In some embodiments, the P23H recognition sequence is SEQ ID NO: 1 and the first subunit comprises residues 198-344 of any one of SEQ ID NOs: 6-69 or residues 7-153 of any one of SEQ ID NOs: 70-93. In some embodiments, the P23H recognition sequence is SEQ ID NO: 1 and the second subunit comprises residues 7-153 of any one of SEQ ID NOs: 6-69 or residues 198-344 of any one of SEQ ID NOs: 70-93.

In some specific embodiments, the P23H recognition sequence is SEQ ID NO: 1 and the meganuclease comprises the amino acid sequence of any one of SEQ ID NOs: 6-93.

In some embodiments, the recombinant meganuclease preferentially recognizes and cleaves one of the P23H recognition sequences (SEQ ID NOs: 1-4) relative to the corresponding 22 base pair recognition sequence present in the wild-type RHO allele (e.g., SE ID NO: 1 relative to SEQ ID NO:5).

In another aspect, the invention provides an isolated polynucleotide comprising a nucleic acid sequence encoding a recombinant meganuclease described herein.

In another aspect, the invention provides a recombinant DNA construct comprising an isolated polynucleotide, wherein the isolated polynucleotide comprises a nucleic acid sequence encoding a recombinant meganuclease described herein. In some embodiments, the recombinant DNA construct encodes a viral vector. In some such embodiments, the viral vector can be a retroviral vector, a lentiviral vector, an adenoviral vector, or an adeno-associated viral (AAV) vector. In some particular embodiment, the recombinant DNA construct encodes a recombinant AAV vector.

In another aspect, the invention provides a viral vector comprising an isolated polynucleotide, wherein the isolated polynucleotide comprises a nucleic acid sequence encoding a recombinant meganuclease described herein. In some embodiments, the viral vector can be a retroviral vector, a lentiviral vector, an adenoviral vector, or an AAV vector. In some particular embodiments, the viral vector can be a recombinant AAV vector.

In another aspect, the invention provides a pharmaceutical composition for treatment of a subject having RP, preferably autosomal dominant RP caused by the P23H mutation. The pharmaceutical composition can comprise a pharmaceutically acceptable carrier and: (a) a nucleic acid encoding a recombinant meganuclease described herein, wherein the recombinant meganuclease is expressed in a target cell in vivo; or (b) a recombinant meganuclease protein described herein; wherein the recombinant meganuclease has specificity for one of the P23H recognition sequences (SEQ ID NOs: 1-4) in the target cell.

In some embodiments, the nucleic acid encoding the recombinant meganuclease can be an mRNA.

In other embodiments, the pharmaceutical composition comprises a recombinant DNA construct comprising the nucleic acid.

In some embodiments, the pharmaceutical composition comprises a viral vector comprising the nucleic acid. In one such embodiment, the viral vector can be a retroviral vector, a lentiviral vector, an adenoviral vector, or an AAV vector. In some particular embodiments, the viral vector can be a recombinant AAV vector.

In another aspect, the invention provides a recombinant meganuclease described herein for use as a medicament. The invention further provides the use of a recombinant meganuclease described herein in the manufacture of a medicament for treating RP, preferably autosomal dominant RP caused by the P23H mutation, and preferably for a subject that is heterozygous for the P23H mutant allele and a functional, normal or wild-type allele.

In another aspect, the invention provides an isolated polynucleotide for use as a medicament, wherein the isolated polynucleotide comprises a nucleic acid sequence encoding a recombinant meganuclease described herein. The invention further provides the use of an isolated polynucleotide in the manufacture of a medicament for treating RP, preferably autosomal dominant RP caused by the P23H mutation, and preferably for a subject that is heterozygous for the P23H mutant allele and a functional, normal or wild-type allele, wherein the isolated polynucleotide comprises a nucleic acid sequence encoding a recombinant meganuclease described herein.

In another aspect, the invention provides a recombinant AAV vector for use as a medicament, wherein the recombinant AAV vector comprises an isolated polynucleotide, and wherein the isolated polynucleotide comprises a nucleic acid sequence encoding a recombinant meganuclease described herein. The invention further provides the use of a recombinant AAV vector in the manufacture of a medicament for treating RP, preferably autosomal dominant RP caused by the P23H mutation, and preferably for a subject that is heterozygous for the P23H mutant allele and a functional, normal or wild-type allele wherein the recombinant AAV vector comprises an isolated polynucleotide, and wherein the isolated polynucleotide comprises a nucleic acid sequence encoding a recombinant meganuclease described herein.

In another aspect, the invention provides a method for treating RP caused by the P23H mutation in a subject in need thereof, and preferably for a subject that is heterozygous for the P23H mutant allele and a functional, normal or wild-type allele. The method comprises contacting the DNA of a target cell of said subject with a recombinant meganuclease described herein that recognizes and cleaves the a recognition sequence including the P23H mutation (e.g, SEQ ID NOs: 1-4). The target cell of the subject comprises the P23H recognition sequence in a RHO gene allele, and cleavage of the recognition sequence inhibits expression of the RHO gene allele.

In some embodiments, the method is for treating autosomal dominant RP caused by the P23H mutation, and preferably for a subject that is heterozygous for the P23H mutant allele and a functional, normal or wild-type allele.

In some embodiments, the target cell in the subject is a retinal target cell. In preferred embodiments, the retinal cell is a rod photoreceptor cell.

In some embodiments of the method, a nucleic acid encoding the recombinant meganuclease is introduced into the target cell. In some such embodiments, the nucleic acid can be an mRNA. In other such embodiments, the nucleic acid can be introduced into the cell using a recombinant DNA construct. In yet other such embodiments, the nucleic acid can be introduced into the target cell using a viral vector such as a retroviral vector, a lentiviral vector, an adenoviral vector, or an AAV vector. In some particular embodiment, a gene encoding the recombinant meganuclease is delivered to the target cell using a recombinant AAV vector.

In other embodiments of the method, a recombinant meganuclease protein of the invention is introduced into the target cell.

In the various embodiments of the method, the recombinant meganuclease protein, or a nucleic acid encoding the recombinant meganuclease, can be administered to the subject in a pharmaceutical composition described herein.

In another aspect, the invention provides a method for producing a genetically-modified cell. The method comprises: (a) obtaining a cell comprising at least one P23H RHO allele; and (b) introducing into the cell: (i) a nucleic acid sequence encoding a recombinant meganuclease of the invention, wherein the recombinant meganuclease is expressed in the cell; or (ii) a recombinant meganuclease protein; wherein the recombinant meganuclease has specificity for a P23H recognition sequence present on the P23H RHO allele(s); and wherein the recombinant meganuclease recognizes and cleaves the P23H recognition sequence; and wherein expression of the P23H RHO allele is disrupted by non-homologous end joining at the cleavage site. Preferably the cell is heterozygous for the P23H mutant allele and a functional, normal or wild-type allele prior to the modification.

In some embodiments of the method, the cell can be a eukaryotic cell. In some such embodiments, the eukaryotic cell can be a pluripotent cell. In such embodiments, the pluripotent cell can be an induced pluripotent stem (iPS) cell. In some particular embodiments, the iPS cell can be a human iPS cell.

In other embodiments of the method, the nucleic acid can be an mRNA.

In some embodiment of the method, the nucleic acid can be introduced into the cell using a recombinant DNA construct.

In some embodiments of the method, the nucleic acid can be introduced into the cell using a viral vector. In some such embodiments, the viral vector can be a retroviral vector, a lentiviral vector, an adenoviral vector, or an AAV vector. In some particular embodiments, the viral vector can be a recombinant AAV vector.

In another aspect, the invention provides a genetically-modified cell, wherein the genetically-modified cell comprises a wild-type RHO allele and a disrupted P23H allele, wherein the genetically-modified cell expresses a wild-type RHO protein and does not express a P23H RHO protein, and wherein the genetically-modified cell is produced according to the methods of the invention described herein. In some embodiments, the disrupted P23H allele includes a deletion mutation caused by cleavage with the meganuclease and NHEJ. In some particular embodiments, the genetically-modified cell can be a pluripotent cell, an iPS cell, or a human iPS cell.

In another aspect, the invention provides a pharmaceutical composition for treatment of a subject having RP caused by the P23H mutation, and preferably for a subject that is heterozygous for the P23H mutant allele and a functional, normal or wild-type allele. In different embodiments, the pharmaceutical composition can comprise a pharmaceutically acceptable carrier and any genetically-modified cell of the invention, and/or any genetically-modified cell produced according to the methods of the invention, which comprises a wild-type RHO allele that expresses wild-type RHO protein and a disrupted P23H allele which does not express P23H RHO protein. In some embodiments, the disrupted P23H allele includes a deletion mutation caused by cleavage with the meganuclease and NHEJ.

In another aspect, the invention provides a method for treating RP caused by the P23H mutation in a subject in need thereof, and preferably for a subject that is heterozygous for the P23H mutant allele and a functional, normal or wild-type allele. The method comprises administering to the subject a pharmaceutical composition described herein which comprises a pharmaceutically acceptable carrier and a genetically-modified cell of the invention, which comprises a wild-type RHO allele that expresses wild-type RHO protein and a disrupted P23H allele which does not express P23H RHO protein. In some embodiments, the disrupted P23H allele includes a deletion mutation caused by cleavage with the meganuclease and NHEJ.

In some embodiments of the method, the genetically-modified cell can be delivered to a target tissue. Such target tissues can include the eye, and particularly the retina.

Further, in some embodiments of the method, the genetically-modified cell can be a genetically-modified iPS cell. In such embodiments, the genetically-modified iPS cell can differentiate into a cell which expresses wild-type RHO protein when it is delivered to the target tissue. In some particular embodiments, the genetically-modified iPS cell can differentiate into a retinal cell, and particularly into a rod photoreceptor cell, which expresses the wild-type rhodopsin protein but not the P23H rhodopsin protein.

### BRIEF DESCRIPTION OF THE FIGURES

FIG. 1. P23H recognition sequence. A) Alignment of the 22 base pair P23H recognition sequence of SEQ ID NO: 1 with the corresponding 22 base pair recognition sequence present in the wild-type human RHO gene allele (SEQ ID NO:5). These sequences span nucleotides 49 to 70 of the P23H mutant or wild-type RHO gene coding sequences (SEQ ID NOs: 98 and 97, respectively). The C68A nucleotide substitution within the P23H recognition sequence is highlighted. B) The P23H recognition sequence of SEQ ID NO: 1 comprises two recognition half-sites, referred to as RHO1 and RHO2. Each recognition half-site comprises 9 base pairs as shown. Half-sites in the recognition sequence are separated by a 4 base pair central region. C) The recombinant meganuclease of the invention comprises two subunits, wherein the first subunit binds to the a first half-site (e.g., RHO1) of a P23H recognition sequence (e.g., SEQ ID NO: 1) and the second subunit binds to a second half-site (e.g., RHO2). In embodiments where the recombinant meganuclease is a single-chain meganuclease, the first subunit can be positioned as either the N-terminal or C-terminal subunit. Likewise, the second subunit can be positioned as either the N-terminal or C-terminal subunit.

FIGS. 2A-2F. Amino acid alignment of RHO1-binding subunits. A-F) Recombinant meganucleases encompassed by the invention comprise one subunit that binds the 9 base pair RHO1 recognition half-site of SEQ ID NO:1. Amino acid sequence alignments are provided for the RHO1-binding subunits (SEQ ID NOs: 102-188) of the recombinant meganucleases set forth in SEQ ID NOs: 6-93. As shown, the RHO1-binding subunit of SEQ ID NOs: 6-69 comprises residues 198-344, whereas the RHO1-binding subunit of SEQ ID NOs: 70-93 comprises residues 7-153. Each RHO1-binding subunit comprises a 56 amino acid hypervariable region as indicated. Variable residues within the hypervariable region are shaded, with the most frequent amino acids at each position further highlighted; the most prevalent residues are bolded, whereas the second most prevalent are bolded and italicized. Residues outside of the hypervariable region are identical in each subunit (but this is not required), with the exception of a Q or E residue at position 80 or position 271 (see, U.S. Pat. No. 8,021,867). Nearly all RHO1-binding subunits provided in FIG. 2A-2F share at least 90% sequence identity to the RHO1-binding subunit (residues 198-344) of the RHO2-L3-59 meganuclease (SEQ ID NO:102). Residue numbers shown are those of SEQ ID NOs: 6-93.

FIGS. 3A-3F. Amino acid alignment of RHO2-binding subunits. A-F) Recombinant meganucleases encompassed by the invention comprise one subunit that binds the 9 base pair RHO2 recognition half-site of SEQ ID NO:1. Amino acid sequence alignments are provided for the RHO2-binding subunits (SEQ ID NOs: 190-277) of the recombinant meganucleases set forth in SEQ ID NOs: 6-93. As shown, the RHO2-binding subunit of SEQ ID NOs: 6-69 comprises residues 7-153, whereas the RHO2-binding subunit of SEQ ID NOs: 70-93 comprises residues 198-344. Each RHO2-binding subunit comprises a 56 amino acid hypervariable region as indicated. Variable residues within the hypervariable region are shaded, with the most frequent amino acids at each position further highlighted; the most prevalent residues are bolded, whereas the second most prevalent are bolded and italicized. Residues outside of the hypervariable region are identical in each subunit (but this is not required), with the exception of a Q or E residue at position 80 or position 271 (see, U.S. Pat. No. 8,021,867). Nearly all RHO2-binding subunits provided in FIGS. 3A-3F share at least 90% sequence identity to the RHO2-binding subunit (residues 7-153) of the RHO2-L3-59 meganuclease (SEQ ID NO: 190). Residue numbers shown are those of SEQ ID NOs: 6-93.

FIG. 4. Schematic of reporter assay in CHO cells for evaluating recombinant meganucleases targeting a P23H recognition sequence. For the recombinant meganucleases described herein, a CHO cell line was produced in which a reporter cassette was integrated stably into the genome of the cell. The reporter cassette comprised, in 5′ to 3′ order: an SV40 Early Promoter; the 5′ ⅔ of the GFP gene; the recognition sequence for an engineered meganuclease of the invention, for example a P23H recognition sequence of any one of SEQ ID NOs: 1-4; the recognition sequence for the CHO-23/24 meganuclease (WO 2012/167192); and the 3′ ⅔ of the GFP gene. Cells stably transfected with this cassette did not express GFP in the absence of a DNA break-inducing agent. Meganucleases were introduced by transduction of plasmid DNA or mRNA encoding each meganuclease. When a DNA break was induced at either of the meganuclease recognition sequences, the duplicated regions of the GFP gene recombined with one another to produce a functional GFP gene. The percentage of GFP-expressing cells could then be determined by flow cytometry as an indirect measure of the frequency of genome cleavage by the meganucleases.

FIG. 5. Efficiency of recombinant meganucleases for recognizing and cleaving a P23H recognition sequence in a CHO cell reporter assay. A)-J) Each of the recombinant meganucleases set forth in SEQ ID NOs: 6-93 were engineered to target a P23H recognition sequence and were screened for efficacy in the CHO cell reporter assay. The results shown provide the percentage of GFP-expressing cells observed in each assay, which indicates the efficacy of each meganuclease for cleaving the P23H recognition sequence or the CHO-23/24 recognition sequence. A negative control (RHO 1-2 bs) was further included in each assay.

FIG. 6. Time course of recombinant meganuclease efficacy in CHO cell reporter assay. Recombinant meganucleases encompassed by the invention were evaluated in the CHO reporter assay, with the percentage of GFP-expressing cells determined 1, 4, 6, and 8 days after introduction of meganuclease-encoding mRNA into the CHO reporter cells. A CHO-23/24 meganuclease was included at each time point as a positive control.

FIG. 7. Selectivity of recombinant meganucleases. A)-F) The selectivity of recombinant meganucleases for a P23H recognition sequence (SEQ ID NO:1) versus the corresponding wild-type RHO recognition sequence (SEQ ID NO:5) was determined using the CHO cell reporter assay. Recombinant meganucleases encompassed by the invention were introduced into cells comprising a P23H recognition sequence (“RHO 1-2 cells,” gray bars) or the corresponding wild-type RHO recognition sequence (“RHO 3-4 cells,” black bars) to determine if they could discriminate between the mutant and wild-type targets.

FIG. 8. Generation and expression of recombinant AAV vectors. A) Diagram of recombinant AAV vector genome shown with inverted terminal repeats (ITRs) at the 5′ and 3′ ends. The vector comprises the coding sequence for the recombinant meganuclease RHO-1/2-L2-49 (SEQ ID NO:8) operably linked to a cytomegalovirus-early (CMV) promoter. The nuclease expression cassette was incorporated into a “packaging” plasmid that was used in conjunction with an Ad helper plasmid to produce recombinant AAV capable of delivering genes encoding the RHO-1/2-L2-49 meganuclease. B) Immunoblot of RHO-1/2-L2-49 meganuclease expression in recombinant AAV-transduced CHO cells after 24 hours.

FIG. 9. Efficiency and selectivity of a RHO-1/2-L2-49 meganuclease for a P23H recognition sequence (SEQ ID NO:1) when expressed by recombinant AAV in a CHO cell reporter assay. CHO cells harboring the wild-type recognition sequence (SEQ ID NO:5; “RHO 3-4 cells”, black bars) or the P23H recognition sequence (SEQ ID NO: 1; “RHO 1-2 cells,” gray bars) were transduced with three different doses of AAV2 vector encoding the RHO-1/2-L2-49 meganuclease operably linked to a CMV promoter.

FIG. 10. RHO-1/2-L2-49 meganuclease stability in CHO reporter cells following treatment with cycloheximide.

FIG. 11. A) Vector map of the pDS CMV RHO2_L3_59 plasmid (SEQ ID NO: 278). B) Vector map of the pDS CMV RHO2_L5_14 plasmid (SEQ ID NO:279).

FIG. 12. CHO GFFP reporter assay demonstrating specificity of RHO 1-2 meganucleases for RHO 1-2 recognition sequence. CHO-K cells (controls), WT RHO cells (comprising wild-type RHO sequence of SEQ ID NO:5), or P23H RHO cells (comprising P23H RHO sequence of SEQ ID NO: 1) were transduced with low, medium, or high titers of recombinant AAV vectors encoding a GFP protein, the RHO2-L3-59 meganuclease (SEQ ID NO: 6), or the RHO2-L5-14 meganuclease (SEQ ID NO:7). The percent of GFP-positive cells was determined in each cell line following transduction as a measure of recognition sequence cleavage.

FIG. 13. Western blot analysis of meganuclease expression in CHO RHO 1-2 cells following transduction of recombinant AAV vectors. P23H RHO cells, comprising the RHO 1-2 recognition sequence, were transduced with 1e8, 1e9, or 2e9 viral genomes per cell of recombinant AAV vectors encoding the RHO2-L3-59 meganuclease (SEQ ID NO:6) or the RHO2-L5-14 meganuclease (SEQ ID NO:7). Cell lysates were analyzed for expression of GFP as a measure of RHO 1-2 recognition sequence cleavage (top panel), meganuclease expression (bottom panel), and β-actin as a loading control. Lanes 1-6 were transduced to express the RHO2-L3-59 meganuclease. Lanes 7-12 were transduced to express the RHO2-L5-14 meganuclease.

FIG. 14. Vector map of the pDS GRKI RHO2_L5_14 plasmid (SEQ ID NO:281).

FIG. 15. Western blot analysis of meganuclease expression in mouse retinal cells following subretinal AAV injection. Wild-type mice were administered a recombinant AAV vector encoding the RHO2-L5-14 meganuclease (SEQ ID NO:7) by subretinal injection. Retinal cells were obtained from the left (OS) and right (OD) eye of five mice and cell lysates were analyzed by Western blot for the expression of the RHO2-L5-14 meganuclease and β-actin.

### BRIEF DESCRIPTION OF THE SEQUENCES

SEQ ID NO: 1 sets forth the nucleotide sequence of one P23H recognition sequence (RHO-1/2).

SEQ ID NO:2 sets forth the nucleotide sequence of one P23H recognition sequence (RHO-9/10).

SEQ ID NO:3 sets forth the nucleotide sequence of one P23H recognition sequence (RHO-11/12).

SEQ ID NO:4 sets forth the nucleotide sequence of one P23H recognition sequence (RHO-13/14).

SEQ ID NO:5 sets forth the nucleotide sequence of the corresponding recognition sequence found in the wild-type RHO allele (i.e., the RHO 3-4 recognition sequence).

SEQ ID NO: 6 sets forth the amino acid sequence of the RHO2-L3-59 meganuclease.

SEQ ID NO: 7 sets forth the amino acid sequence of the RHO2-L5-14 meganuclease.

SEQ ID NO: 8 sets forth the amino acid sequence of the RHO-1/2-L2-49 meganuclease.

SEQ ID NO: 9 sets forth the amino acid sequence of the RHO 1-2x.179 meganuclease.

SEQ ID NO: 10 sets forth the amino acid sequence of the RHO 1-2x.4 meganuclease. SEQ ID NO: 11 sets forth the amino acid sequence of the RHO 1-2x.207 meganuclease.

SEQ ID NO: 12 sets forth the amino acid sequence of the RHO 1-2x.277 meganuclease.

SEQ ID NO: 13 sets forth the amino acid sequence of the RHO 1-2x.292 meganuclease.

SEQ ID NO: 14 sets forth the amino acid sequence of the RHO 1-2x.324 meganuclease.

SEQ ID NO: 15 sets forth the amino acid sequence of the RHO 1-2x.371 meganuclease.

SEQ ID NO: 16 sets forth the amino acid sequence of the RHO 1-2x.164 meganuclease.

SEQ ID NO: 17 sets forth the amino acid sequence of the RHO 1-2x.181 meganuclease.

SEQ ID NO: 18 sets forth the amino acid sequence of the RHO 1-2x.184 meganuclease.

SEQ ID NO: 19 sets forth the amino acid sequence of the RHO-1/2-L1-21 meganuclease.

SEQ ID NO: 20 sets forth the amino acid sequence of the RHO-1/2-L1-43 meganuclease.

SEQ ID NO: 21 sets forth the amino acid sequence of the RHO-1/2-L1-45 meganuclease.

SEQ ID NO: 22 sets forth the amino acid sequence of the RHO-1/2-L1-60 meganuclease.

SEQ ID NO: 23 sets forth the amino acid sequence of the RHO-1/2-L1-61 meganuclease.

SEQ ID NO: 24 sets forth the amino acid sequence of the RHO-1/2-L1-58 meganuclease.

SEQ ID NO: 25 sets forth the amino acid sequence of the RHO-1/2-L1-7 meganuclease.

SEQ ID NO: 26 sets forth the amino acid sequence of the RHO-1/2-L1-13 meganuclease.

SEQ ID NO: 27 sets forth the amino acid sequence of the RHO-1/2-L1-18 meganuclease.

SEQ ID NO: 28 sets forth the amino acid sequence of the RHO-1/2-L1-70 meganuclease.

SEQ ID NO: 29 sets forth the amino acid sequence of the RHO-1/2-L1-86 meganuclease.

SEQ ID NO: 30 sets forth the amino acid sequence of the RHO-1/2-L2-13 meganuclease.

SEQ ID NO: 31 sets forth the amino acid sequence of the RHO-1/2-L2-24 meganuclease.

SEQ ID NO: 32 sets forth the amino acid sequence of the RHO-1/2-L2-37 meganuclease.

SEQ ID NO: 33 sets forth the amino acid sequence of the RHO-1/2-L2-58 meganuclease.

SEQ ID NO: 34 sets forth the amino acid sequence of the RHO-1/2-L2-31 meganuclease.

SEQ ID NO: 35 sets forth the amino acid sequence of the RHO-1/2-L2-29 meganuclease.

SEQ ID NO: 36 sets forth the amino acid sequence of the RHO-1/2-L2-61 meganuclease.

SEQ ID NO: 37 sets forth the amino acid sequence of the RHO2-L3-2 meganuclease.

SEQ ID NO: 38 sets forth the amino acid sequence of the RHO2-L3-3 meganuclease.

SEQ ID NO: 39 sets forth the amino acid sequence of the RHO2-L3-5 meganuclease.

SEQ ID NO: 40 sets forth the amino acid sequence of the RHO2-L3-10 meganuclease.

SEQ ID NO: 41 sets forth the amino acid sequence of the RHO2-L3-11 meganuclease.

SEQ ID NO: 42 sets forth the amino acid sequence of the RHO2-L3-12 meganuclease.

SEQ ID NO: 43 sets forth the amino acid sequence of the RHO2-L3-13 meganuclease.

SEQ ID NO: 44 sets forth the amino acid sequence of the RHO2-L3-28 meganuclease.

SEQ ID NO: 45 sets forth the amino acid sequence of the RHO2-L3-29 meganuclease.

SEQ ID NO: 46 sets forth the amino acid sequence of the RHO2-L3-57 meganuclease.

SEQ ID NO: 47 sets forth the amino acid sequence of the RHO2-L3-80 meganuclease.

SEQ ID NO: 48 sets forth the amino acid sequence of the RHO2-L3-85 meganuclease.

SEQ ID NO: 49 sets forth the amino acid sequence of the RHO2-L3-86 meganuclease.

SEQ ID NO: 50 sets forth the amino acid sequence of the RHO2-L3-92 meganuclease.

SEQ ID NO: 51 sets forth the amino acid sequence of the RHO2-L3-4 meganuclease.

SEQ ID NO: 52 sets forth the amino acid sequence of the RHO2-L3-20 meganuclease.

SEQ ID NO: 53 sets forth the amino acid sequence of the RHO2-L3-72 meganuclease.

SEQ ID NO: 54 sets forth the amino acid sequence of the RHO1-L1-4 meganuclease.

SEQ ID NO: 55 sets forth the amino acid sequence of the RHO1-L1-8 meganuclease.

SEQ ID NO: 56 sets forth the amino acid sequence of the RHO1-L1-13 meganuclease.

SEQ ID NO: 57 sets forth the amino acid sequence of the RHO1-L1-19 meganuclease.

SEQ ID NO: 58 sets forth the amino acid sequence of the RHO1-L1-58 meganuclease.

SEQ ID NO: 59 sets forth the amino acid sequence of the RHO1-L1-69 meganuclease.

SEQ ID NO: 60 sets forth the amino acid sequence of the RHO1-L1-80 meganuclease.

SEQ ID NO: 61 sets forth the amino acid sequence of the RHO1-L1-82 meganuclease.

SEQ ID NO: 62 sets forth the amino acid sequence of the RHO1-L1-73 meganuclease.

SEQ ID NO: 63 sets forth the amino acid sequence of the RHO1-L1-85 meganuclease.

SEQ ID NO: 64 sets forth the amino acid sequence of the RHO1-L1-86 meganuclease.

SEQ ID NO: 65 sets forth the amino acid sequence of the RHO-1/2-LA-10 meganuclease.

SEQ ID NO: 66 sets forth the amino acid sequence of the RHO-1/2-LA-29 meganuclease.

SEQ ID NO: 67 sets forth the amino acid sequence of the RHO-1/2-LA-65 meganuclease.

SEQ ID NO: 68 sets forth the amino acid sequence of the RHO-1/2-LA-66 meganuclease.

SEQ ID NO: 69 sets forth the amino acid sequence of the RHO-1/2-LA-85 meganuclease.

SEQ ID NO: 70 sets forth the amino acid sequence of the RHO 1-2x.216 meganuclease.

SEQ ID NO: 71 sets forth the amino acid sequence of the RHO 1-2x.241 meganuclease.

SEQ ID NO: 72 sets forth the amino acid sequence of the RHO 1-2x.94 meganuclease.

SEQ ID NO: 73 sets forth the amino acid sequence of the RHO 1-2x.95 meganuclease.

SEQ ID NO: 74 sets forth the amino acid sequence of the RHO 1-2x.1 meganuclease.

SEQ ID NO: 75 sets forth the amino acid sequence of the RHO 1-2x.60 meganuclease.

SEQ ID NO: 76 sets forth the amino acid sequence of the RHO 1-2x.74 meganuclease.

SEQ ID NO: 77 sets forth the amino acid sequence of the RHO 1-2x.88 meganuclease.

SEQ ID NO: 78 sets forth the amino acid sequence of the RHO 1-2x.294 meganuclease.

SEQ ID NO: 79 sets forth the amino acid sequence of the RHO 1-2x.302 meganuclease.

SEQ ID NO: 80 sets forth the amino acid sequence of the RHO 1-2x.306 meganuclease.

SEQ ID NO: 81 sets forth the amino acid sequence of the RHO 1-2x.338 meganuclease.

SEQ ID NO: 82 sets forth the amino acid sequence of the RHO 1-2x.348 meganuclease.

SEQ ID NO: 83 sets forth the amino acid sequence of the RHO 1-2x.356 meganuclease.

SEQ ID NO: 84 sets forth the amino acid sequence of the RHO 1-2x.364 meganuclease.

SEQ ID NO: 85 sets forth the amino acid sequence of the RHO 1-2x.142 meganuclease.

SEQ ID NO: 86 sets forth the amino acid sequence of the RHO 1-2x.177 meganuclease.

SEQ ID NO: 87 sets forth the amino acid sequence of the RHO 1-2x.148 meganuclease.

SEQ ID NO: 88 sets forth the amino acid sequence of the RHO 1-2x.20 meganuclease.

SEQ ID NO: 89 sets forth the amino acid sequence of the RHO 1-2x.55 meganuclease.

SEQ ID NO: 90 sets forth the amino acid sequence of the RHO 1-2x.197 meganuclease.

SEQ ID NO: 91 sets forth the amino acid sequence of the RHO 1-2x.252 meganuclease.

SEQ ID NO: 92 sets forth the amino acid sequence of the RHO 1-2x.372 meganuclease.

SEQ ID NO: 93 sets forth the amino acid sequence of the RHO 1-2x.151 meganuclease.

SEQ ID NO:94 sets forth the amino acid sequence of the wild-type I-CreI meganuclease.

SEQ ID NO:95 sets forth the amino acid sequence of the LAGLIDADG motif of the

I-CreI meganuclease.

SEQ ID NO:96 sets forth the nucleic acid sequence of the coding region for wild-type human rhodopsin.

SEQ ID NO:97 sets forth the nucleic acid sequence of the coding region for mutant P23H rhodopsin.

SEQ ID NO:98 sets forth the nucleic acid sequence of the wild-type human rhodopsin gene.

SEQ ID NO:99 sets forth the nucleic acid sequence of the human rhodopsin gene comprising a C68A mutation that encodes a P23H substitution in rhodopsin.

SEQ ID NO: 100 sets forth the amino acid sequence of wild-type human rhodopsin.

SEQ ID NO:101 sets for the amino acid sequence of mutant P23H rhodopsin.

SEQ ID NO: 102 sets forth residues 198-344 of the RHO2-L3-59 meganuclease.

SEQ ID NO: 103 sets forth residues 198-344 of the RHO2-L5-14 meganuclease.

SEQ ID NO: 104 sets forth residues 198-344 of the RHO-1/2-L2-49 meganuclease.

SEQ ID NO: 105 sets forth residues 198-344 of the RHO 1-2x.179 meganuclease.

SEQ ID NO: 106 sets forth residues 198-344 of the RHO 1-2x.4 meganuclease.

SEQ ID NO: 107 sets forth residues 198-344 of the RHO 1-2x.207 meganuclease.

SEQ ID NO: 108 sets forth residues 198-344 of the RHO 1-2x.277 meganuclease.

SEQ ID NO: 109 sets forth residues 198-344 of the RHO 1-2x.292 meganuclease.

SEQ ID NO: 110 sets forth residues 198-344 of the RHO 1-2x.324 meganuclease.

SEQ ID NO: 111 sets forth residues 198-344 of the RHO 1-2x.371 meganuclease.

SEQ ID NO: 112 sets forth residues 198-344 of the RHO 1-2x.164 meganuclease.

SEQ ID NO: 113 sets forth residues 198-344 of the RHO 1-2x.181 meganuclease.

SEQ ID NO: 114 sets forth residues 198-344 of the RHO 1-2x.184 meganuclease.

SEQ ID NO: 115 sets forth residues 198-344 of the RHO-1/2-L1-21 meganuclease.

SEQ ID NO: 116 sets forth residues 198-344 of the RHO-1/2-L1-43 meganuclease.

SEQ ID NO: 117 sets forth residues 198-344 of the RHO-1/2-L1-45 meganuclease.

SEQ ID NO: 118 sets forth residues 198-344 of the RHO-1/2-L1-60 meganuclease.

SEQ ID NO: 119 sets forth residues 198-344 of the RHO-1/2-L1-61 meganuclease.

SEQ ID NO: 120 sets forth residues 198-344 of the RHO-1/2-L1-58 meganuclease.

SEQ ID NO: 121 sets forth residues 198-344 of the RHO-1/2-L1-7 meganuclease.

SEQ ID NO: 122 sets forth residues 198-344 of the RHO-1/2-L1-13 meganuclease.

SEQ ID NO: 123 sets forth residues 198-344 of the RHO-1/2-L1-18 meganuclease. SEQ ID NO: 124 sets forth residues 198-344 of the RHO-1/2-L1-70 meganuclease.

SEQ ID NO: 125 sets forth residues 198-344 of the RHO-1/2-L1-86 meganuclease.

SEQ ID NO: 126 sets forth residues 198-344 of the RHO-1/2-L2-13 meganuclease. SEQ ID NO: 127 sets forth residues 198-344 of the RHO-1/2-L2-24 meganuclease.

SEQ ID NO: 128 sets forth residues 198-344 of the RHO-1/2-L2-37 meganuclease. SEQ ID NO: 129 sets forth residues 198-344 of the RHO-1/2-L2-58 meganuclease.

SEQ ID NO: 130 sets forth residues 198-344 of the RHO-1/2-L2-31 meganuclease.

SEQ ID NO: 131 sets forth residues 198-344 of the RHO-1/2-L2-29 meganuclease. SEQ ID NO: 132 sets forth residues 198-344 of the RHO-1/2-L2-61 meganuclease.

SEQ ID NO: 133 sets forth residues 198-344 of the RHO2-L3-2 meganuclease.

SEQ ID NO: 134 sets forth residues 198-344 of the RHO2-L3-3 meganuclease.

SEQ ID NO: 135 sets forth residues 198-344 of the RHO2-L3-5 meganuclease.

SEQ ID NO: 136 sets forth residues 198-344 of the RHO2-L3-10 meganuclease.

SEQ ID NO: 137 sets forth residues 198-344 of the RHO2-L3-11 meganuclease.

SEQ ID NO: 138 sets forth residues 198-344 of the RHO2-L3-12 meganuclease.

SEQ ID NO: 139 sets forth residues 198-344 of the RHO2-L3-13 meganuclease.

SEQ ID NO: 140 sets forth residues 198-344 of the RHO2-L3-28 meganuclease.

SEQ ID NO: 141 sets forth residues 198-344 of the RHO2-L3-29 meganuclease.

SEQ ID NO: 142 sets forth residues 198-344 of the RHO2-L3-57 meganuclease.

SEQ ID NO: 143 sets forth residues 198-344 of the RHO2-L3-80 meganuclease.

SEQ ID NO: 144 sets forth residues 198-344 of the RHO2-L3-85 meganuclease.

SEQ ID NO: 145 sets forth residues 198-344 of the RHO2-L3-86 meganuclease.

SEQ ID NO: 146 sets forth residues 198-344 of the RHO2-L3-92 meganuclease.

SEQ ID NO: 147 sets forth residues 198-344 of the RHO2-L3-4 meganuclease.

SEQ ID NO: 148 sets forth residues 198-344 of the RHO2-L3-20 meganuclease.

SEQ ID NO: 149 sets forth residues 198-344 of the RHO2-L3-72 meganuclease.

SEQ ID NO: 150 sets forth residues 198-344 of the RHO1-L1-4 meganuclease.

SEQ ID NO: 151 sets forth residues 198-344 of the RHO1-L1-8 meganuclease.

SEQ ID NO: 152 sets forth residues 198-344 of the RHO1-L1-13 meganuclease.

SEQ ID NO: 153 sets forth residues 198-344 of the RHO1-L1-19 meganuclease.

SEQ ID NO: 154 sets forth residues 198-344 of the RHO1-L1-58 meganuclease.

SEQ ID NO: 155 sets forth residues 198-344 of the RHO1-L1-69 meganuclease.

SEQ ID NO: 156 sets forth residues 198-344 of the RHO1-L1-80 meganuclease.

SEQ ID NO: 157 sets forth residues 198-344 of the RHO1-L1-82 meganuclease.

SEQ ID NO: 158 sets forth residues 198-344 of the RHO1-L1-73 meganuclease.

SEQ ID NO: 159 sets forth residues 198-344 of the RHO1-L1-85 meganuclease.

SEQ ID NO: 160 sets forth residues 198-344 of the RHO1-L1-86 meganuclease.

SEQ ID NO: 161 sets forth residues 198-344 of the RHO-1/2-L4-10 meganuclease. SEQ ID NO: 162 sets forth residues 198-344 of the RHO-1/2-L4-29 meganuclease.

SEQ ID NO: 163 sets forth residues 198-344 of the RHO-1/2-L4-65 meganuclease.

SEQ ID NO: 164 sets forth residues 198-344 of the RHO-1/2-L4-66 meganuclease.

SEQ ID NO: 165 sets forth residues 198-344 of the RHO-1/2-LA-85 meganuclease.

SEQ ID NO: 166 sets forth residues 7-153 of the RHO 1-2x.216 meganuclease.

SEQ ID NO: 167 sets forth residues 7-153 of the RHO 1-2x.241 meganuclease.

SEQ ID NO: 168 sets forth residues 7-153 of the RHO 1-2x.94 meganuclease.

SEQ ID NO: 169 sets forth residues 7-153 of the RHO 1-2x.95 meganuclease.

SEQ ID NO: 170 sets forth residues 7-153 of the RHO 1-2x. 1 meganuclease.

SEQ ID NO: 171 sets forth residues 7-153 of the RHO 1-2x.60 meganuclease.

SEQ ID NO: 172 sets forth residues 7-153 of the RHO 1-2x.74 meganuclease.

SEQ ID NO: 173 sets forth residues 7-153 of the RHO 1-2x.88 meganuclease.

SEQ ID NO: 174 sets forth residues 7-153 of the RHO 1-2x.294 meganuclease.

SEQ ID NO: 175 sets forth residues 7-153 of the RHO 1-2x.302 meganuclease.

SEQ ID NO: 176 sets forth residues 7-153 of the RHO 1-2x.306 meganuclease.

SEQ ID NO: 177 sets forth residues 7-153 of the RHO 1-2x.338 meganuclease.

SEQ ID NO: 178 sets forth residues 7-153 of the RHO 1-2x.348 meganuclease.

SEQ ID NO: 179 sets forth residues 7-153 of the RHO 1-2x.356 meganuclease.

SEQ ID NO: 180 sets forth residues 7-153 of the RHO 1-2x.364 meganuclease.

SEQ ID NO: 181 sets forth residues 7-153 of the RHO 1-2x.142 meganuclease.

SEQ ID NO: 182 sets forth residues 7-153 of the RHO 1-2x. 177 meganuclease.

SEQ ID NO: 183 sets forth residues 7-153 of the RHO 1-2x.148 meganuclease.

SEQ ID NO: 184 sets forth residues 7-153 of the RHO 1-2x.20 meganuclease.

SEQ ID NO: 185 sets forth residues 7-153 of the RHO 1-2x.55 meganuclease.

SEQ ID NO: 186 sets forth residues 7-153 of the RHO 1-2x.197 meganuclease.

SEQ ID NO: 187 sets forth residues 7-153 of the RHO 1-2x.252 meganuclease.

SEQ ID NO: 188 sets forth residues 7-153 of the RHO 1-2x.372 meganuclease.

SEQ ID NO: 189 sets forth residues 7-153 of the RHO 1-2x. 151 meganuclease.

SEQ ID NO: 190 sets forth residues 7-153 of the RHO2-L3-59 meganuclease.

SEQ ID NO: 191 sets forth residues 7-153 of the RHO2-L5-14 meganuclease.

SEQ ID NO: 192 sets forth residues 7-153 of the RHO-1/2-L2-49 meganuclease.

SEQ ID NO: 193 sets forth residues 7-153 of the RHO 1-2x.179 meganuclease.

SEQ ID NO: 194 sets forth residues 7-153 of the RHO 1-2x.4 meganuclease.

SEQ ID NO: 195 sets forth residues 7-153 of the RHO 1-2x.207 meganuclease.

SEQ ID NO: 196 sets forth residues 7-153 of the RHO 1-2x.277 meganuclease.

SEQ ID NO: 197 sets forth residues 7-153 of the RHO 1-2x.292 meganuclease.

SEQ ID NO: 198 sets forth residues 7-153 of the RHO 1-2x.324 meganuclease.

SEQ ID NO: 199 sets forth residues 7-153 of the RHO 1-2x.371 meganuclease.

SEQ ID NO: 200 sets forth residues 7-153 of the RHO 1-2x.164 meganuclease.

SEQ ID NO: 201 sets forth residues 7-153 of the RHO 1-2x.181 meganuclease.

SEQ ID NO: 202 sets forth residues 7-153 of the RHO 1-2x.184 meganuclease.

SEQ ID NO: 203 sets forth residues 7-153 of the RHO-1/2-L1-21 meganuclease.

SEQ ID NO: 204 sets forth residues 7-153 of the RHO-1/2-L1-43 meganuclease.

SEQ ID NO: 205 sets forth residues 7-153 of the RHO-1/2-L1-45 meganuclease.

SEQ ID NO: 206 sets forth residues 7-153 of the RHO-1/2-L1-60 meganuclease.

SEQ ID NO: 207 sets forth residues 7-153 of the RHO-1/2-L1-61 meganuclease.

SEQ ID NO: 208 sets forth residues 7-153 of the RHO-1/2-L1-58 meganuclease.

SEQ ID NO: 209 sets forth residues 7-153 of the RHO-1/2-L1-7 meganuclease.

SEQ ID NO: 210 sets forth residues 7-153 of the RHO-1/2-L1-13 meganuclease.

SEQ ID NO: 211 sets forth residues 7-153 of the RHO-1/2-L1-18 meganuclease.

SEQ ID NO: 212 sets forth residues 7-153 of the RHO-1/2-L1-70 meganuclease.

SEQ ID NO: 213 sets forth residues 7-153 of the RHO-1/2-L 1-86 meganuclease.

SEQ ID NO: 214 sets forth residues 7-153 of the RHO-1/2-L2-13 meganuclease.

SEQ ID NO: 215 sets forth residues 7-153 of the RHO-1/2-L2-24 meganuclease.

SEQ ID NO: 216 sets forth residues 7-153 of the RHO-1/2-L2-37 meganuclease.

SEQ ID NO: 217 sets forth residues 7-153 of the RHO-1/2-L2-58 meganuclease.

SEQ ID NO: 218 sets forth residues 7-153 of the RHO-1/2-L2-31 meganuclease.

SEQ ID NO: 219 sets forth residues 7-153 of the RHO-1/2-L2-29 meganuclease.

SEQ ID NO: 220 sets forth residues 7-153 of the RHO-1/2-L2-61 meganuclease.

SEQ ID NO: 221 sets forth residues 7-153 of the RHO2-L3-2 meganuclease.

SEQ ID NO: 222 sets forth residues 7-153 of the RHO2-L3-3 meganuclease.

SEQ ID NO: 223 sets forth residues 7-153 of the RHO2-L3-5 meganuclease.

SEQ ID NO: 224 sets forth residues 7-153 of the RHO2-L3-10 meganuclease.

SEQ ID NO: 225 sets forth residues 7-153 of the RHO2-L3-11 meganuclease.

SEQ ID NO: 226 sets forth residues 7-153 of the RHO2-L3-12 meganuclease.

SEQ ID NO: 227 sets forth residues 7-153 of the RHO2-L3-13 meganuclease.

SEQ ID NO: 228 sets forth residues 7-153 of the RHO2-L3-28 meganuclease.

SEQ ID NO: 229 sets forth residues 7-153 of the RHO2-L3-29 meganuclease.

SEQ ID NO: 230 sets forth residues 7-153 of the RHO2-L3-57 meganuclease.

SEQ ID NO: 231 sets forth residues 7-153 of the RHO2-L3-80 meganuclease.

SEQ ID NO: 232 sets forth residues 7-153 of the RHO2-L3-85 meganuclease.

SEQ ID NO: 233 sets forth residues 7-153 of the RHO2-L3-86 meganuclease.

SEQ ID NO: 234 sets forth residues 7-153 of the RHO2-L3-92 meganuclease.

SEQ ID NO: 235 sets forth residues 7-153 of the RHO2-L3-4 meganuclease.

SEQ ID NO: 236 sets forth residues 7-153 of the RHO2-L3-20 meganuclease.

SEQ ID NO: 237 sets forth residues 7-153 of the RHO2-L3-72 meganuclease.

SEQ ID NO: 238 sets forth residues 7-153 of the RHO1-L1-4 meganuclease.

SEQ ID NO: 239 sets forth residues 7-153 of the RHO1-L1-8 meganuclease.

SEQ ID NO: 240 sets forth residues 7-153 of the RHO1-L1-13 meganuclease.

SEQ ID NO: 241 sets forth residues 7-153 of the RHO1-L1-19 meganuclease.

SEQ ID NO: 242 sets forth residues 7-153 of the RHO1-L1-58 meganuclease.

SEQ ID NO: 243 sets forth residues 7-153 of the RHO1-L1-69 meganuclease.

SEQ ID NO: 244 sets forth residues 7-153 of the RHO1-L1-80 meganuclease.

SEQ ID NO: 245 sets forth residues 7-153 of the RHO1-L1-82 meganuclease.

SEQ ID NO: 246 sets forth residues 7-153 of the RHO1-L1-73 meganuclease.

SEQ ID NO: 244 sets forth residues 7-153 of the RHO1-L1-85 meganuclease.

SEQ ID NO: 248 sets forth residues 7-153 of the RHO1-L1-86 meganuclease.

SEQ ID NO: 249 sets forth residues 7-153 of the RHO-1/2-L4-10 meganuclease.

SEQ ID NO: 250 sets forth residues 7-153 of the RHO-1/2-L4-29 meganuclease.

SEQ ID NO: 251 sets forth residues 7-153 of the RHO-1/2-L4-65 meganuclease.

SEQ ID NO: 252 sets forth residues 7-153 of the RHO-1/2-L4-66 meganuclease.

SEQ ID NO: 253 sets forth residues 7-153 of the RHO-1/2-L4-85 meganuclease.

SEQ ID NO: 254 sets forth residues 198-344 of the RHO 1-2x.216 meganuclease.

SEQ ID NO: 255 sets forth residues 198-344 of the RHO 1-2x.241 meganuclease.

SEQ ID NO: 256 sets forth residues 198-344 of the RHO 1-2x.94 meganuclease.

SEQ ID NO: 257 sets forth residues 198-344 of the RHO 1-2x.95 meganuclease.

SEQ ID NO: 258 sets forth residues 198-344 of the RHO 1-2x.1 meganuclease.

SEQ ID NO: 259 sets forth residues 198-344 of the RHO 1-2x.60 meganuclease.

SEQ ID NO:260 sets forth residues 198-344 of the RHO 1-2x.74 meganuclease.

SEQ ID NO: 261 sets forth residues 198-344 of the RHO 1-2x.88 meganuclease.

SEQ ID NO: 262 sets forth residues 198-344 of the RHO 1-2x.294 meganuclease.

SEQ ID NO: 263 sets forth residues 198-344 of the RHO 1-2x.302 meganuclease.

SEQ ID NO: 264 sets forth residues 198-344 of the RHO 1-2x.306 meganuclease.

SEQ ID NO: 265 sets forth residues 198-344 of the RHO 1-2x.338 meganuclease.

SEQ ID NO: 266 sets forth residues 198-344 of the RHO 1-2x.348 meganuclease.

SEQ ID NO: 267 sets forth residues 198-344 of the RHO 1-2x.356 meganuclease.

SEQ ID NO: 268 sets forth residues 198-344 of the RHO 1-2x.364 meganuclease.

SEQ ID NO: 269 sets forth residues 198-344 of the RHO 1-2x.142 meganuclease.

SEQ ID NO: 270 sets forth residues 198-344 of the RHO 1-2x.177 meganuclease.

SEQ ID NO: 271 sets forth residues 198-344 of the RHO 1-2x.148 meganuclease.

SEQ ID NO: 272 sets forth residues 198-344 of the RHO 1-2x.20 meganuclease.

SEQ ID NO: 273 sets forth residues 198-344 of the RHO 1-2x.55 meganuclease.

SEQ ID NO: 274 sets forth residues 198-344 of the RHO 1-2x.197 meganuclease.

SEQ ID NO: 275 sets forth residues 198-344 of the RHO 1-2x.252 meganuclease.

SEQ ID NO: 276 sets forth residues 198-344 of the RHO 1-2x.372 meganuclease.

SEQ ID NO: 277 sets forth residues 198-344 of the RHO 1-2x. 151 meganuclease.

SEQ ID NO: 278 sets forth the nucleic acid sequence of the pDS CMV RHO2_L3_59 plasmid.

SEQ ID NO: 279 sets forth the nucleic acid sequence of the pDS CMV RHO2_L5_14 plasmid.

SEQ ID NO: 280 sets forth the nucleic acid sequence of the pDS GRK1 RHO2_L3_59 plasmid.

SEQ ID NO: 281 sets forth the nucleic acid sequence of the pDS GRKI RHO2_LS_14 plasmid.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “meganuclease” refers to an endonuclease that binds double-stranded DNA at a recognition sequence that is greater than 12 base pairs. Preferably, the recognition sequence for a meganuclease of the invention is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859). A meganuclease as used herein binds to double-stranded DNA as a heterodimer. A meganuclease may also be a “single-chain meganuclease” in which a pair of DNA-binding domains are joined into a single polypeptide using a peptide linker.

As used herein, the term “single-chain meganuclease” refers to a polypeptide comprising a pair of meganuclease subunits joined by a linker. A single-chain meganuclease has the organization: N-terminal subunit-Linker-C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease. Methods for producing single-chain meganuclease variants of I-CreI are known in the art (e.g., WO 2009/059195; Li, et al. (2009) Nucleic Acids Res. 37:1650-62; Grizot, et al. (2009) Nucleic Acids Res. 37:5405-19). The term “homing endonuclease” is synonymous with the term “meganuclease.”

As used herein, the term “linker” refers to an exogenous peptide sequence used to join two meganuclease subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. No. 8,445,251. In some embodiments, a linker may have an amino acid sequence comprising residues 154-195 of any one of SEQ ID NOs: 6-93.

As used herein, with respect to a protein, the term “recombinant” means having an altered amino acid sequence as a result of the application of genetic engineering techniques to nucleic acids which encode the protein, and cells or organisms which express the protein. With respect to a nucleic acid, the term “recombinant” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. The mutant RHO P23H allele and the P23H rhodopsin protein are distinguishable from the wild-type RHO allele and rhodopsin protein. Further, wild-type homing endonucleases are distinguishable from recombinant or non-naturally-occurring meganucleases.

As used herein with respect to recombinant proteins, the term “modification” means any insertion, deletion or substitution of an amino acid residue in the recombinant sequence relative to a reference sequence (e.g., a wild-type or a native sequence).

As used herein, the term “recognition sequence” refers to a DNA sequence that is bound and cleaved by a meganuclease. In the case of a recombinant meganuclease of the invention, a recognition sequence comprises a pair of inverted, 9 base pair “half-sites” or “recognition half-sites” which are separated by four base pairs. In the case of a single-chain meganuclease, the N-terminal subunit of the protein contacts a first half-site and the C-terminal subunit of the protein contacts a second half-site. Cleavage by a meganuclease produces four base pair 3′ “overhangs”. “Overhangs”, or “sticky ends” are short, single-stranded DNA segments that can be produced by meganuclease cleavage of a double-stranded DNA sequence. In the case of meganucleases of the present invention, the overhang comprises bases 10-13 of the 22 base pair recognition sequence.

As used herein, the term “target site” or “target sequence” refers to a region of the chromosomal DNA of a cell comprising a recognition sequence for a meganuclease.

As used herein, the term “DNA-binding affinity” or “binding affinity” means the tendency of a meganuclease to non-covalently associate with a reference DNA molecule (e.g., a recognition sequence or an arbitrary sequence). Binding affinity is measured by a dissociation constant, K_{d}. As used herein, a meganuclease has “altered” binding affinity if the K_{d} of the recombinant meganuclease for a reference recognition sequence is increased or decreased by a statistically significant (p<0.05) amount relative to a reference meganuclease.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases such as meganucleases can be used to effectively knock-out a gene in a population of cells.

As used herein with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7 (1-2): 203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3. Similarly, the percent identity can be established based upon an alignment of the sequences which maximizes identity between aligned amino acid residues or nucleotides, and which is a function of the number of identical residues or nucleotides divided by the number of total residues or nucleotides in the larger of the two sequences.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first proteins corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, the term “recognition half-site,” “recognition sequence half-site,” or simply “half-site” means a nucleic acid sequence in a double-stranded DNA molecule which is recognized by a monomer of a homodimeric or heterodimeric meganuclease, or by one subunit of a single-chain meganuclease.

As used herein, the term “preferentially” refers to the specificity of a recombinant meganuclease for recognizing and cleaving a particular target recognition sequence in the genome relative to a second, reference recognition sequence. By way of example, a recombinant meganuclease of the invention may preferentially recognize and cleave a P23H recognition sequence (e.g., SEQ ID NO: 1) with greater efficiency than it recognizes and cleaves the corresponding wild-type recognition sequence (e.g., SEQ ID NO: 5), as determined by methods known in the art, including those methods provided in the examples herein. In some embodiments, a recombinant meganuclease of the invention may recognize and cleave a P23H recognition sequence with greater than about 5%, 10%, 15%, 20%, 25%, 30%, 40%, 50%, 60%, 70%, 80%, 90%, or 100% more efficiency than it recognizes and cleaves the corresponding wild-type recognition sequence. In other embodiments, a recombinant meganuclease of the invention may recognize and cleave a P23H recognition sequence with greater than about 1-fold, 2-fold, 5-fold, 10-fold, 50-fold, 100-fold, or 1000-fold more efficiency than it recognizes and cleaves the corresponding wild-type recognition sequence.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of any one of SEQ ID NOs: 6-93. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-18 residues that exhibit variability and can be modified to influence base preference and/or DNA-binding affinity. In some embodiments, variable residues within a hypervariable region correspond to one or more of positions 24, 26, 28, 29, 30, 32, 33, 38, 39, 40, 42, 44, 46, 68, 70, 73, 75, and 77 of any one of SEQ ID NOs: 6-93. In other embodiments, variable residues within a hypervariable region correspond to one or more of positions 215, 217, 219, 220, 221, 223, 224, 229, 230, 231, 233, 235, 237, 259, 261, 264, 266, and 268 of any one of SEQ ID NOs: 6-93.

As used herein, the terms “RHO,” “RHO gene,” “rhodopsin gene,” and “wild-type RHO allele” are used interchangeably and refer to the human rhodopsin gene, preferably the gene identified by NCBI Reference Sequence NG_009115.1 (SEQ ID NO: 98). The terms “mutant RHO allele” and “mutant RHO P23H allele” are used interchangeably and refer to a RHO allele sequence comprising a C68A mutation (SEQ ID NO: 99), which results in a P23H substitution in the encoded protein. The terms “rhodopsin” and “wild-type rhodopsin” are used interchangeably and refer to the protein encoded by the wild-type rhodopsin gene, particularly the protein identified by NCBI Reference Sequence NP_000530.1 (SEQ ID NO: 100). The term “P23H rhodopsin” refers to the mutant rhodopsin protein comprising a P23H substitution, particularly the protein set forth in SEQ ID NO: 101.

The terms “recombinant DNA construct,” “recombinant construct,” “expression cassette,” “expression construct,” “chimeric construct,” “construct,” and “recombinant DNA fragment” are used interchangeably herein and are nucleic acid fragments. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in that art suitable for delivering a gene encoding a meganuclease of the invention to a target cell. The skilled artisan is well aware of the genetic elements that must be present on the vector in order to successfully transform, select and propagate host cells comprising any of the isolated nucleotides or nucleic acid sequences of the invention.

As used herein, a “vector” can also refer to a viral vector. Viral vectors can include, without limitation, retroviral vectors, lentiviral vectors, adenoviral vectors, and adeno-associated viral vectors (AAV).

As used herein, a “target cell” refers to a cell that comprises at least one RHO allele comprising a P23H recognition sequence (e.g., one of SEQ ID NOs: 1-4). Such target cells can express mutant RHO P23H protein. Target cells can include, without limitation, cells of the eye, preferably cells in the posterior segment of the eye, and even more preferably cells of the retina, including rod photoreceptor cells comprising a P23H recognition sequence in at least one RHO gene allele.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first proteins corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, the terms “treatment” or “treating a subject” refers to the administration of recombinant meganuclease of the invention, or a nucleic acid encoding recombinant meganuclease of the invention, to a subject having RP for the purpose of providing partial or complete relief of one or more symptoms of RP. In some aspects, recombinant meganuclease of the invention, or a nucleic acid encoding the same, is administered during treatment in the form of a pharmaceutical composition of the invention. Preferably the subject is heterozygous for the P23H mutant allele and a functional, normal or wild-type allele.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and 2≤if the variable is inherently continuous.

### 2.1 Principle of Targeting and Inactivating the Mutant RHO P23H Allele

The present invention is based, in part, on the hypothesis that autosomal dominant RP can be corrected or prevented by targeting, cleaving, and inactivating a mutant RHO P23H allele, which encodes the pathogenic P23H rhodopsin protein. Surprisingly, recombinant meganucleases can be engineered to recognize and cleave a P23H recognition sequence (e.g., one of SEQ ID NOs: 1-4) present in the mutant RHO P23H allele. Such recombinant meganucleases can preferentially target and cleave the mutant RHO P23H allele relative to the corresponding wild-type allele (SEQ ID NO:96). NHEJ at the cleavage site results in mutagenesis and disruption of the mutant RHO P23H allele, while the functional wild-type RHO allele remains intact to express wild-type rhodopsin in rod photoreceptor cells of the retina. Preferential inactivation of the mutant RHO P23H allele, and disruption of P23H rhodopsin expression, is expected to prevent, delay, or reverse the progression of RP in patients.

### 2.2 Meganucleases for Recognizing and Cleaving the P23H Recognition Sequence

Meganucleases can make a site-specific DNA break in the genome of a living cell, and such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of meganucleases to induce mutagenesis via NHEJ can be used to target a specific mutation or a sequence present in a wild-type allele.

In preferred embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs.

Recombinant meganucleases of the invention have been engineered to recognize and cleave a P23H recognition sequence (e.g., one of SEQ ID NOs: 1-4). Such meganucleases preferentially cleave the P23H recognition sequence on the mutant RHO P23H allele relative to the corresponding wild-type RHO recognition sequence (SEQ ID NO: 96). Exemplary recombinant meganucleases of the invention are provided in SEQ ID NOs: 6-93, which are collectively referred to herein as “RHO 1-2 meganucleases” or “RHO 1/2 meganucleases.

Recombinant meganucleases of the invention comprise a first subunit, comprising a first hypervariable (HVR1) region, and a second subunit, comprising a second hypervariable (HVR2) region. Further, the first subunit binds to a first recognition half-site in a P23H recognition sequence (e.g., the RHO1 half-site), and the second subunit binds to a second recognition half-site in the P23H recognition sequence (e.g., the RHO2 half-site). In embodiments where the recombinant meganuclease is a single-chain meganuclease, the first and second subunits can be oriented such that the first subunit is positioned as the N-terminal subunit, and the second subunit is positioned as the C-terminal subunit (e.g., SEQ ID NOs: 70-93). In alternative embodiments, the first and second subunits can be oriented such that the first subunit is positioned as the C-terminal subunit, and the second subunit is positioned as the N-terminal subunit (e.g., SEQ ID NOs: 6-69). Exemplary recombinant meganucleases of the invention are provided in Table 1.

TABLE 1
Exemplary recombinant meganucleases engineered to recognize
and cleave the P23H recognition sequence (SEQ ID NO: 1)

| | | RHO1 | RHO1 | *RHO1 | RHO2 | RHO2 | *RHO2 |
|---|---|---|---|---|---|---|---|
| | AA | Subunit | Subunit | Subunit | Subunit | Subunit | Subunit |
| Meganuclease | SEQ ID | Residues | SEQ ID | % | Residues | SEQ ID | % |
| RHO2-L3-59 | 6 | 198-344 | 102 | 100 | 7-153 | 187 | 100 |
| RHO2-L5-14 | 7 | 198-344 | 103 | 100 | 7-153 | 188 | 96.6 |
| RHO-1/2-L2-49 | 8 | 198-344 | 104 | 100 | 7-153 | 189 | 96.6 |
| RHO 1-2x.179 | 9 | 198-344 | 105 | 100 | 7-153 | 190 | 93.2 |
| RHO 1-2x.4 | 10 | 198-344 | 106 | 92.52 | 7-153 | 191 | 91.16 |
| RHO 1-2x.207 | 11 | 198-344 | 107 | 93.2 | 7-153 | 192 | 91.84 |
| RHO 1-2x.277 | 12 | 198-344 | 108 | 93.2 | 7-153 | 193 | 91.84 |
| RHO 1-2x.292 | 13 | 198-344 | 109 | 94.56 | 7-153 | 194 | 90.48 |
| RHO 1-2x.324 | 14 | 198-344 | 110 | 92.52 | 7-153 | 195 | 91.16 |
| RHO 1-2x.371 | 15 | 198-344 | 111 | 93.2 | 7-153 | 196 | 91.16 |
| RHO 1-2x.164 | 16 | 198-344 | 112 | 91.16 | 7-153 | 197 | 90.48 |
| RHO 1-2x.181 | 17 | 198-344 | 113 | 95.24 | 7-153 | 198 | 90.48 |
| RHO 1-2x.184 | 18 | 198-344 | 114 | 90.48 | 7-153 | 199 | 88.44 |
| RHO-1/2-L1-21 | 19 | 198-344 | 115 | 100 | 7-153 | 200 | 93.2 |
| RHO-1/2-L1-43 | 20 | 198-344 | 116 | 100 | 7-153 | 201 | 92.52 |
| RHO-1/2-L1-45 | 21 | 198-344 | 117 | 100 | 7-153 | 202 | 92.52 |
| RHO-1/2-L1-60 | 22 | 198-344 | 118 | 100 | 7-153 | 203 | 92.52 |
| RHO-1/2-L1-61 | 23 | 198-344 | 119 | 100 | 7-153 | 204 | 92.52 |
| RHO-1/2-L1-58 | 24 | 198-344 | 120 | 100 | 7-153 | 205 | 92.52 |
| RHO-1/2-L1-7 | 25 | 198-344 | 121 | 100 | 7-153 | 206 | 92.52 |
| RHO-1/2-L1-13 | 26 | 198-344 | 122 | 100 | 7-153 | 207 | 92.52 |
| RHO-1/2-L1-18 | 27 | 198-344 | 123 | 100 | 7-153 | 208 | 92.52 |
| RHO-1/2-L1-70 | 28 | 198-344 | 124 | 100 | 7-153 | 209 | 92.52 |
| RHO-1/2-L1-86 | 29 | 198-344 | 125 | 100 | 7-153 | 210 | 92.52 |
| RHO-1/2-L2-13 | 30 | 198-344 | 126 | 100 | 7-153 | 211 | 95.24 |
| RHO-1/2-L2-24 | 31 | 198-344 | 127 | 100 | 7-153 | 212 | 95.24 |
| RHO-1/2-L2-37 | 32 | 198-344 | 128 | 100 | 7-153 | 213 | 93.88 |
| RHO-1/2-L2-58 | 33 | 198-344 | 129 | 100 | 7-153 | 214 | 93.2 |
| RHO-1/2-L2-31 | 34 | 198-344 | 130 | 100 | 7-153 | 215 | 93.2 |
| RHO-1/2-L2-29 | 35 | 198-344 | 131 | 100 | 7-153 | 216 | 93.2 |
| RHO-1/2-L2-61 | 36 | 198-344 | 132 | 100 | 7-153 | 217 | 93.88 |
| RHO2-L3-2 | 37 | 198-344 | 133 | 100 | 7-153 | 218 | 94.56 |
| RHO2-L3-3 | 38 | 198-344 | 134 | 100 | 7-153 | 219 | 95.24 |
| RHO2-L3-5 | 39 | 198-344 | 135 | 100 | 7-153 | 220 | 96.6 |
| RHO2-L3-10 | 40 | 198-344 | 136 | 100 | 7-153 | 221 | 94.56 |
| RHO2-L3-11 | 41 | 198-344 | 137 | 100 | 7-153 | 222 | 95.24 |
| RHO2-L3-12 | 42 | 198-344 | 138 | 100 | 7-153 | 223 | 95.24 |
| RHO2-L3-13 | 43 | 198-344 | 139 | 100 | 7-153 | 224 | 95.24 |
| RHO2-L3-28 | 44 | 198-344 | 140 | 100 | 7-153 | 225 | 95.24 |
| RHO2-L3-29 | 45 | 198-344 | 141 | 100 | 7-153 | 226 | 94.56 |
| RHO2-L3-57 | 46 | 198-344 | 142 | 100 | 7-153 | 227 | 95.24 |
| RHO2-L3-80 | 47 | 198-344 | 143 | 100 | 7-153 | 228 | 95.92 |
| RHO2-L3-85 | 48 | 198-344 | 144 | 100 | 7-153 | 229 | 95.24 |
| RHO2-L3-86 | 49 | 198-344 | 145 | 100 | 7-153 | 230 | 94.56 |
| RHO2-L3-92 | 50 | 198-344 | 146 | 100 | 7-153 | 231 | 95.24 |
| RHO2-L3-4 | 51 | 198-344 | 147 | 100 | 7-153 | 232 | 94.56 |
| RHO2-L3-20 | 52 | 198-344 | 148 | 100 | 7-153 | 233 | 94.56 |
| RHO2-L3-72 | 53 | 198-344 | 149 | 100 | 7-153 | 234 | 95.92 |
| RHO1-L1-4 | 54 | 198-344 | 150 | 96.6 | 7-153 | 235 | 96.6 |
| RHO1-L1-8 | 55 | 198-344 | 151 | 95.24 | 7-153 | 236 | 96.6 |
| RHO1-L1-13 | 56 | 198-344 | 152 | 95.24 | 7-153 | 237 | 96.6 |
| RHO1-L1-19 | 57 | 198-344 | 153 | 95.24 | 7-153 | 238 | 96.6 |
| RHO1-L1-58 | 58 | 198-344 | 154 | 95.92 | 7-153 | 239 | 96.6 |
| RHO1-L1-69 | 59 | 198-344 | 155 | 95.92 | 7-153 | 240 | 96.6 |
| RHO1-L1-80 | 60 | 198-344 | 156 | 95.92 | 7-153 | 241 | 96.6 |
| RHO1-L1-82 | 61 | 198-344 | 157 | 95.92 | 7-153 | 245 | 96.6 |
| RHO1-L1-73 | 62 | 198-344 | 158 | 96.6 | 7-153 | 246 | 96.6 |
| RHO1-L1-85 | 63 | 198-344 | 159 | 96.6 | 7-153 | 247 | 96.6 |
| RHO1-L1-86 | 64 | 198-344 | 160 | 96.6 | 7-153 | 248 | 96.6 |
| RHO-1/2-L4-10 | 65 | 198-344 | 161 | 95.24 | 7-153 | 249 | 95.24 |
| RHO-1/2-L4-29 | 66 | 198-344 | 162 | 95.24 | 7-153 | 250 | 95.24 |
| RHO-1/2-L4-65 | 67 | 198-344 | 163 | 95.92 | 7-153 | 251 | 94.56 |
| RHO-1/2-L4-66 | 68 | 198-344 | 164 | 97.28 | 7-153 | 252 | 95.24 |
| RHO-1/2-L4-85 | 69 | 198-344 | 165 | 96.6 | 7-153 | 253 | 94.56 |
| RHO 1-2x.216 | 70 | 7-153 | 166 | 93.2 | 198-344 | 254 | 91.16 |
| RHO 1-2x.241 | 71 | 7-153 | 167 | 93.88 | 198-344 | 255 | 91.16 |
| RHO 1-2x.94 | 72 | 7-153 | 168 | 91.84 | 198-344 | 256 | 91.16 |
| RHO 1-2x.95 | 73 | 7-153 | 169 | 91.16 | 198-344 | 257 | 91.16 |
| RHO 1-2x.1 | 74 | 7-153 | 170 | 93.2 | 198-344 | 258 | 90.48 |
| RHO 1-2x.60 | 75 | 7-153 | 171 | 93.2 | 198-344 | 259 | 90.48 |
| RHO 1-2x.74 | 76 | 7-153 | 172 | 93.88 | 198-344 | 260 | 90.48 |
| RHO 1-2x.88 | 77 | 7-153 | 173 | 91.16 | 198-344 | 261 | 90.48 |
| RHO 1-2x.294 | 78 | 7-153 | 174 | 93.88 | 198-344 | 262 | 90.48 |
| RHO 1-2x.302 | 79 | 7-153 | 175 | 93.2 | 198-344 | 263 | 90.48 |
| RHO 1-2x.306 | 80 | 7-153 | 176 | 92.52 | 198-344 | 264 | 90.48 |
| RHO 1-2x.338 | 81 | 7-153 | 177 | 93.2 | 198-344 | 265 | 90.48 |
| RHO 1-2x.348 | 82 | 7-153 | 178 | 92.52 | 198-344 | 266 | 90.48 |
| RHO 1-2x.356 | 83 | 7-153 | 179 | 91.16 | 198-344 | 267 | 90.48 |
| RHO 1-2x.364 | 84 | 7-153 | 180 | 93.2 | 198-344 | 268 | 90.48 |
| RHO 1-2x.142 | 85 | 7-153 | 181 | 93.2 | 198-344 | 269 | 90.48 |
| RHO 1-2x.177 | 86 | 7-153 | 182 | 94.56 | 198-344 | 270 | 90.48 |
| RHO 1-2x.148 | 87 | 7-153 | 183 | 91.84 | 198-344 | 271 | 93.2 |
| RHO 1-2x.20 | 88 | 7-153 | 184 | 93.2 | 198-344 | 272 | 89.12 |
| RHO 1-2x.55 | 89 | 7-153 | 185 | 92.52 | 198-344 | 273 | 92.52 |
| RHO 1-2x.197 | 90 | 7-153 | 186 | 95.24 | 198-344 | 274 | 91.84 |
| RHO 1-2x.252 | 91 | 7-153 | 187 | 93.2 | 198-344 | 275 | 88.44 |
| RHO 1-2x.372 | 92 | 7-153 | 188 | 94.56 | 198-344 | 276 | 91.84 |
| RHO 1-2x.151 | 93 | 7-153 | 189 | 94.56 | 198-344 | 277 | 90.48 |

*“RHO1 Subunit %” and “RHO2 Subunit %” represent the amino acid sequence identity between the RHO 1-binding and RHO2-binding subunit regions of each meganuclease and the RHO 1-binding and RHO2-binding subunit regions, respectively, of the RHO2-L3-59 meganuclease.

### 2.3 Methods for Delivering and Expressing Recombinant Meganucleases

Treating RP using the invention requires that a recombinant meganuclease can be expressed in cells in the appropriate tissues. The target tissue(s) for delivery of recombinant meganucleases of the invention are cells of the eye, preferably cells in the posterior segment of the eye, and even more preferably cells of the retina, including rod photoreceptor cells. Recombinant meganucleases can be delivered as purified protein or as RNA or DNA encoding the meganucleases. In one embodiment, recombinant meganuclease proteins, or mRNA or vector encoding recombinant meganucleases, are supplied to target cells (e.g., cells in the retina) via injection directly to the target tissue. For example, delivery of RNA, DNA, or recombinant AAV vectors to the eye via subretinal or intravitreal injection is described in the art (see for example, Martin et al. (2002) Methods. 28:267-275; Hauswirth et al. (2008) Human Gene Therapy. 19 (10): 979-990; Johnson et al. (2008) Molecular Vision. 14:2211-2226). Alternatively, meganuclease protein, mRNA, or DNA can be delivered systemically via the circulatory system.

In some embodiments, recombinant meganuclease proteins, or DNA/mRNA encoding recombinant meganucleases, are formulated for systemic administration, or administration to target tissues, in a pharmaceutical carrier in accordance with known techniques. Sec, e.g., Remington, The Science And Practice of Pharmacy (21^{st} ed. 2005). In the manufacture of a pharmaceutical formulation according to the invention, proteins/RNA/mRNA are typically admixed with a pharmaceutically acceptable carrier. The carrier must, of course, be acceptable in the sense of being compatible with any other ingredients in the formulation and must not be deleterious to the patient. The carrier can be a solid or a liquid, or both, and can be formulated with the compound as a unit-dose formulation.

In some embodiments, recombinant meganuclease proteins, or DNA/mRNA encoding recombinant meganucleases, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med Res. Rev. 25:679-736), MPG (Simeoni, et al. (2003) Nucleic Acids Res. 31:2717-2724), Pep-1 (Deshayes et al. (2004) Biochemistry 43:7698-7706, and HSV-1 VP-22 (Deshayes et al. (2005) Cell Mol Life Sci. 62:1839-49. In an alternative embodiment, recombinant meganucleases, or DNA/mRNA encoding recombinant meganucleases, are coupled covalently or non-covalently to an antibody that recognizes a specific cell-surface receptor expressed on target cells such that the meganuclease protein/DNA/mRNA binds to and is internalized by the target cells. Alternatively, recombinant meganuclease protein/DNA/mRNA can be coupled covalently or non-covalently to the natural ligand (or a portion of the natural ligand) for such a cell-surface receptor. (McCall, et al. (2014) Tissue Barriers. 2 (4): e944449; Dinda, et al. (2013) Curr Pharm Biotechnol. 14:1264-74; Kang, et al. (2014) Curr Pharm Biotechnol. 15 (3): 220-30; Qian et al. (2014) Expert Opin Drug Metah Toxicol. 10 (11): 1491-508). Examples of targeting ligands to direct delivery to cells in the eye include RGD (Pollinger et al. (2013) PNAS. 110 (15): 6115-6120), transferrin (Lajunen et al. (2014) Eur J Pharm Sci. 62:23-32), and hyaluronic acid (Martens et al. (2015) J Control Release. 202:83-92).

In some embodiments, recombinant meganuclease proteins, or DNA/mRNA encoding recombinant meganucleases, are encapsulated within biodegradable hydrogels for injection or implantation within the desired region of the eye (e.g., intravitreal or subconjunctival injection). Hydrogels can provide sustained and tunable release of the therapeutic payload to the desired region of the eye without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, recombinant meganuclease proteins, or DNA/mRNA encoding recombinant meganucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the recombinant meganuclease proteins, mRNA, or DNA can be attached to or encapsulated with the nanoparticle core. This increases the copy number of the protein/mRNA/DNA that is delivered to each cell and, so, increases the intracellular expression of each recombinant meganuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012), Biomaterials. 33 (30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the meganuclease proteins or DNA/mRNA encoding the meganucleases are encapsulated within liposomes or complexed using cationic lipids (see, e.g., Lipofectamine™, Life Technologies Corp., Carlsbad, CA; Zuris et al. (2015), Nat Biotechnol. 33:73-80; Mishra et al. (2011), J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, recombinant meganuclease proteins, or DNA/mRNA encoding recombinant meganucleases, are encapsulated within polymeric scaffolds (e.g., PLGA) or complexed using cationic polymers (e.g., PEI, PLL) (Tamboli et al. (2011), Ther Deliv. 2 (4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, recombinant meganuclease proteins, or DNA/mRNA encoding recombinant meganucleases, are combined with amphiphilic molecules that self-assemble into micelles (Tong et al. (2007), J Gene Med. 9 (11): 956-66). Polymeric micelles may include a micellar shell formed with a hydrophilic polymer (e.g., polyethyleneglycol) that can prevent aggregation, mask charge interactions, and reduce nonspecific interactions within the vitreous fluid.

In some embodiments, recombinant meganuclease proteins, or DNA/mRNA encoding recombinant meganucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in US Patent Application Nos. 2002/0045667 and 2004/0043041, and U.S. Pat. Nos. 6,015,832, 6,506,803, 6,635,676, and 6,559,189, each of which is incorporated herein by reference in its entirety.

In some embodiments, recombinant meganuclease proteins, or DNA/mRNA encoding recombinant meganucleases, are covalently attached to, or non-covalently associated with, multifunctional polymer conjugates, DNA dendrimers, and polymeric dendrimers (Mastorakos et al. (2015), Nanoscale. 7 (9): 3845-56; Cheng et al. (2008), J Pharm Sci. 97 (1): 123-43). The dendrimer generation can control the payload capacity and size, and can provide a high drug payload capacity. Moreover, display of multiple surface groups can be leveraged to improve stability, reduce nonspecific interactions, and enhance cell-specific targeting and drug release.

In some embodiments, genes encoding a recombinant meganuclease are delivered using a viral vector. Such vectors are known in the art and include lentiviral vectors, adenoviral vectors, and adeno-associated virus (AAV) vectors (reviewed in Vannucci, et al. (2013), New Microbiol. 36:1-22). In some embodiments, the viral vectors are injected directly into target tissues (Bosch, et al. (2000), Mol Ther. 1:63-70; Greig et al. (2014), PLOS One. November 13;9 (11): c112268). In alternative embodiments, the viral vectors are delivered systemically via the circulatory system. It is known in the art that different AAV vectors tend to localize to different tissues. In retinal target tissues, effective transduction of retinal photoreceptor cells has been shown, for example, with AAV serotypes 1, 2, 5, 8, and 9 (Petrs-Silva et al. (2014), Clinical Ophthalmology. 8:127-136). AAV vectors can also be self-complementary such that they do not require second-strand DNA synthesis in the host cell (McCarty et al. (2001), Gene Ther. 8:1248-54).

In one embodiment, a viral vector used for meganuclease gene delivery is a self-limiting viral vector. A self-limiting viral vector can have limited persistence time in a cell or organism due to the presence of a recognition sequence for a recombinant meganuclease within the vector. Thus, a self-limiting viral vector can be engineered to provide coding for a promoter, a recombinant meganuclease described herein, and a meganuclease recognition site within the ITRs. The self-limiting viral vector delivers the meganuclease gene to a cell, tissue, or organism, such that the meganuclease is expressed and able to cut the genome of the cell at an endogenous recognition sequence within the genome. The delivered meganuclease will also find its target site within the self-limiting viral vector itself, and cut the vector at this target site. Once cut, the 5′ and 3′ ends of the viral genome will be exposed and degraded by exonucleases, thus killing the virus and ceasing production of the recombinant meganuclease.

If the recombinant meganuclease genes are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a preferred embodiment, meganuclease genes are operably linked to a promoter that drives gene expression preferentially in the target cells. Examples of retina and/or rod photoreceptor cell-specific promoters include, without limitation, the human rhodopsin kinase promoter, the proximal mouse opsin promoter (mOP), the human G-protein-coupled receptor protein kinase 1 promoter (hGRK1), and the human interphotoreceptor retinoid-binding protein (IRBP) promoter (Khani et al. (2007), Invest. Ophthamol. Vis. Sci. 48 (9): 3954-3961); Beltran et al. (2010), Gene Therapy. 17 (9): 1162-1174); Yokoyama et al (1992), Exp. Eye Res. 55 (2): 225-233), as well as rod photoreceptor cell-specific promoters disclosed in U.S. Patent Publication No. US 2014/0287510.

In some embodiments, the invention provides a pharmaceutical composition comprising a pharmaceutically acceptable carrier and a recombinant meganuclease of the invention, or a pharmaceutically acceptable carrier and an isolated polynucleotide comprising a nucleic acid encoding a recombinant meganuclease of the invention. Such pharmaceutical compositions can be prepared in accordance with known techniques. Sec, e.g., Remington, The Science And Practice of Pharmacy (21^{st} ed. 2005). In the manufacture of a pharmaceutical formulation according to the invention, endonuclease polypeptides (or DNA/RNA encoding the same) are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents or biological molecules useful in the treatment of a disease in the subject. Likewise, the additional agent(s) and/or biological molecule(s) can be co-administered as a separate composition.

It is envisioned that a single treatment will permanently inactivate the mutant P23H RHO allele in a percentage of patient target cells. If the frequency of P23H allele inactivation is low, however, or if a large percentage of target cells need to be corrected, it may be necessary to perform multiple treatments on each patient.

### 2.4 Recombinant Meganuclease Variants

Embodiments of the invention encompass the recombinant meganucleases described herein, and variants thereof. Further embodiments of the invention encompass isolated polynucleotides comprising a nucleic acid sequence encoding the recombinant meganucleases described herein, and variants of such polynucleotides.

In particular, the invention provides variants of the meganucleases of SEQ ID Nos: 6-93 in which the hypervariable regions have been modified such that the meganucleases preferentially recognize and cleave one of SEQ ID NOs: 2-4 relative to the corresponding wild-type sequences in the wild-type RHO gene (SEQ ID NO: 98). Such additional variants meganucleases can be produced by routine experimentation according to the methods described in WO2007/047859, U.S. Pat. Nos. 8,021,867, 8,119,361, 8,119,381, 8,124,369, 8,129,134, 8,133,697, 8,143,015, 8,143,016, 8,148,098, 8,163,514, 8,304,222, 8,377,674 and 8,445,251, and discussed below.

As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein; i.e., the ability to preferentially recognize and cleave a P23H recognition sequence (e.g., one of SEQ ID NOs: 1-4), as described herein. Such variants may result, for example, from human manipulation. Biologically active variants of a native polypeptide of the embodiments (e.g., SEQ ID NOs: 6-93), or biologically active variants of the RHO1- and RHO2-binding subunits described herein (e.g., SEQ ID NOs: 99-274), will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide or native subunit, as determined by sequence alignment programs and parameters described elsewhere herein. A biologically active variant of a polypeptide or subunit of the embodiments may differ from that polypeptide or subunit by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. Sec, for example, Kunkel (1985), Proc. Natl. Acad. Sci. USA 82:488-492; Kunkel et al. (1987), Methods in Enzymol. 154:367-382; U.S. Pat. No. 4,873,192; Walker and Gaastra, eds. (1983), Techniques in Molecular Biology (MacMillan Publishing Company, New York) and the references cited therein. Guidance as to appropriate amino acid substitutions that do not affect biological activity of the protein of interest may be found in the model of Dayhoff et al. (1978), Atlas of Protein Sequence and Structure (Natl. Biomed. Res. Found., Washington, D.C.), herein incorporated by reference. Conservative substitutions, such as exchanging one amino acid with another having similar properties, may be optimal.

A substantial number of amino acid modifications to the DNA recognition domain of the wild-type I-CreI meganuclease have previously been identified (e.g., U.S. Pat. No. 8,021,867) which, singly or in combination, result in recombinant meganucleases with specificities altered at individual bases within the DNA recognition sequence half-site, such that the resulting rationally-designed meganucleases have half-site specificities different from the wild-type enzyme. Table 2 provides potential substitutions that can be made in a recombinant meganuclease monomer or subunit to enhance specificity based on the base present at each half-site position (−1 through −9) of a recognition half-site.

TABLE 2

| | Favored Sense-Strand Base | | | | | | | | | | |
|---|---|---|---|---|---|---|---|---|---|---|---|
| Posn. | A | C | G | T | A/T | A/C | A/G | C/T | G/T | A/G/T | A/C/G/T |

| | | | | | | | | | | |
|---|---|---|---|---|---|---|---|---|---|---|
| −1 | Y75 | R70* | K70 | Q70* | | | | T46* | | G70 |
| | L75* | H75* | E70* | C70 | | | | | | A70 |
| | C75* | R75* | E75* | L70 | | | | | | S70 |
| | Y139* | H46* | E46* | Y75* | | | | | | G46* |
| | C46* | K46* | D46* | Q75* | | | | | | |
| | A46* | R46* | | H75* | | | | | | |
| | | | | H139 | | | | | | |
| | | | | Q46* | | | | | | |
| | | | | H46* | | | | | | |
| −2 | Q70 | E70 | H70 | Q44* | C44* | | | | | |
| | T44* | D70 | D44* | | | | | | | |
| | A44* | K44* | E44* | | | | | | | |
| | V44* | R44* | | | | | | | | |
| | I44* | | | | | | | | | |
| | L44* | | | | | | | | | |
| | N44* | | | | | | | | | |
| −3 | Q68 | E68 | R68 | M68 | | H68 | | Y68 | K68 | |
| | C24* | F68 | | C68 | | | | | | |
| | I24* | K24* | | L68 | | | | | | |
| | | R24* | | F68 | | | | | | |
| −4 | A26* | E77 | R77 | | | | | S77 | | S26* |
| | Q77 | K26* | E26* | | | | | Q26* | | |
| −5 | | E42 | R42 | | | K28* | C28* | | | M66 |
| | | | | | | | Q42 | | | K66 |
| −6 | Q40 | E40 | R40 | C40 | A40 | | | | | S40 |
| | C28* | R28* | | I40 | A79 | | | | | S28* |
| | | | | V40 | A28* | | | | | |
| | | | | C79 | H28* | | | | | |
| | | | | I79 | | | | | | |
| | | | | V79 | | | | | | |
| | | | | Q28* | | | | | | |
| −7 | N30* | E38 | K38 | I38 | | | C38 | | | H38 |
| | Q38 | K30* | R38 | L38 | | | | | | N38 |
| | | R30* | E30* | | | | | | | Q30* |
| −8 | F33 | E33 | F33 | L33 | | R32* | R33 | | | |
| | Y33 | D33 | H33 | V33 | | | | | | |
| | | | | I33 | | | | | | |
| | | | | F33 | | | | | | |
| | | | | C33 | | | | | | |
| −9 | | E32 | R32 | L32 | | | | D32 | | S32 |
| | | | K32 | V32 | | | | I32 | | N32 |
| | | | | A32 | | | | | | H32 |
| | | | | C32 | | | | | | Q32 |
| | | | | | | | | | | T32 |

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a recombinant meganuclease of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its ability to preferentially recognize and cleave a P23H recognition sequence (e.g., one of SEQ ID NOs: 1-4).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Evaluation of Meganucleases that Recognize and Cleave a

### P23H Recognition Sequence

1. Meganucleases that Recognize and Cleave a P23H Recognition Sequence

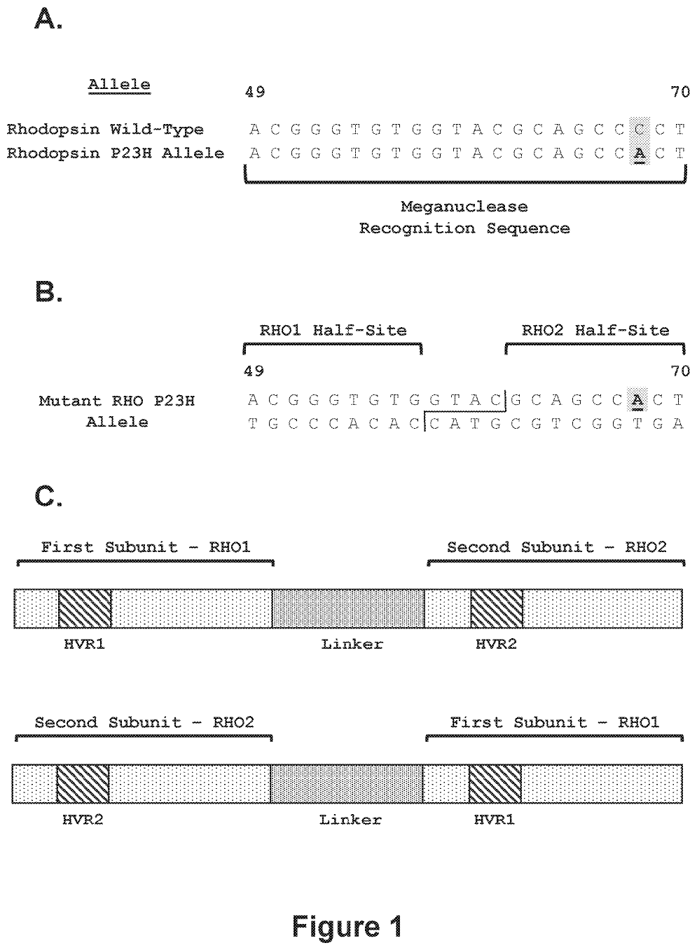

Recombinant meganucleases (SEQ ID NOs: 6-93), collectively referred to herein as “RHO 1-2 meganucleases,” were engineered to recognize and cleave one of the P23H recognition sequences (i.e, SEQ ID NO:1), which is present in the mutant RHO P23H allele (see, FIG. 1A). Each RHO 1-2 recombinant meganuclease comprises an N-terminal nuclease-localization signal derived from SV40, a first meganuclease subunit, a linker sequence, and a second meganuclease subunit. A first subunit in each RHO 1-2 meganuclease binds to the RHO1 recognition half-site of SEQ ID NO:1, while a second subunit binds to the RHO2 recognition half-site (see, FIG. 1B).

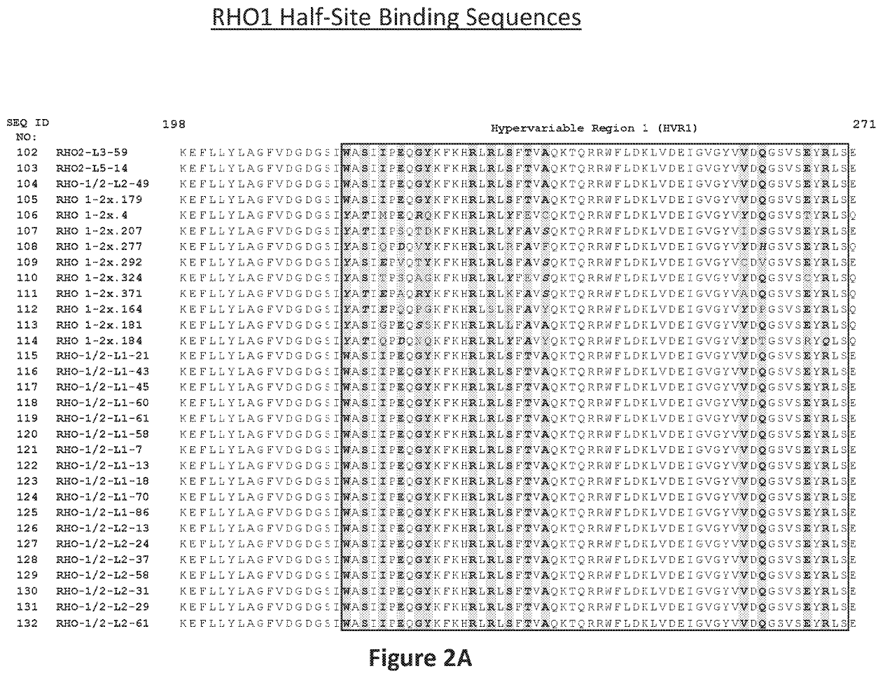

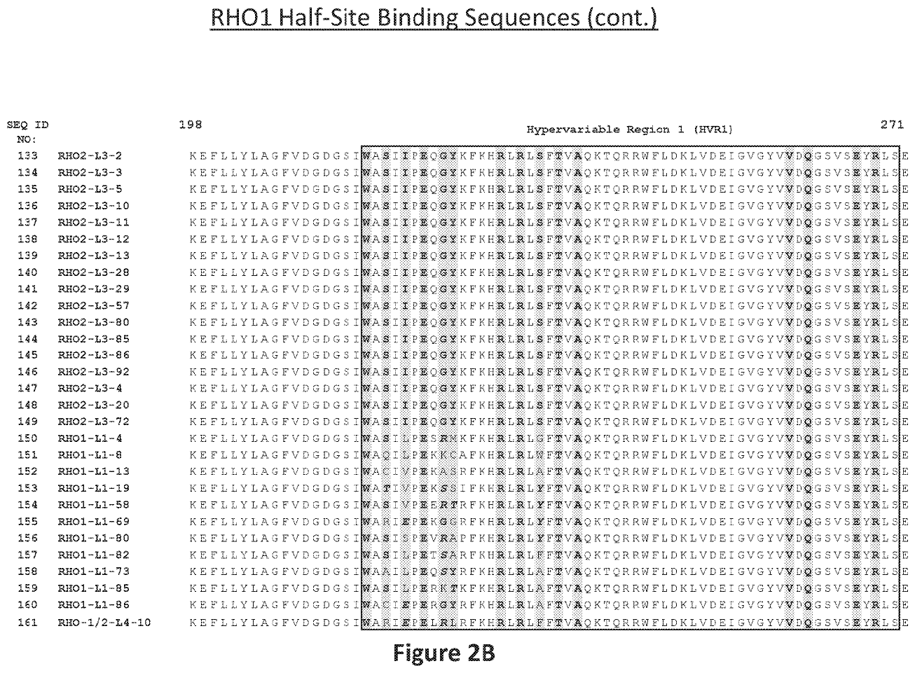

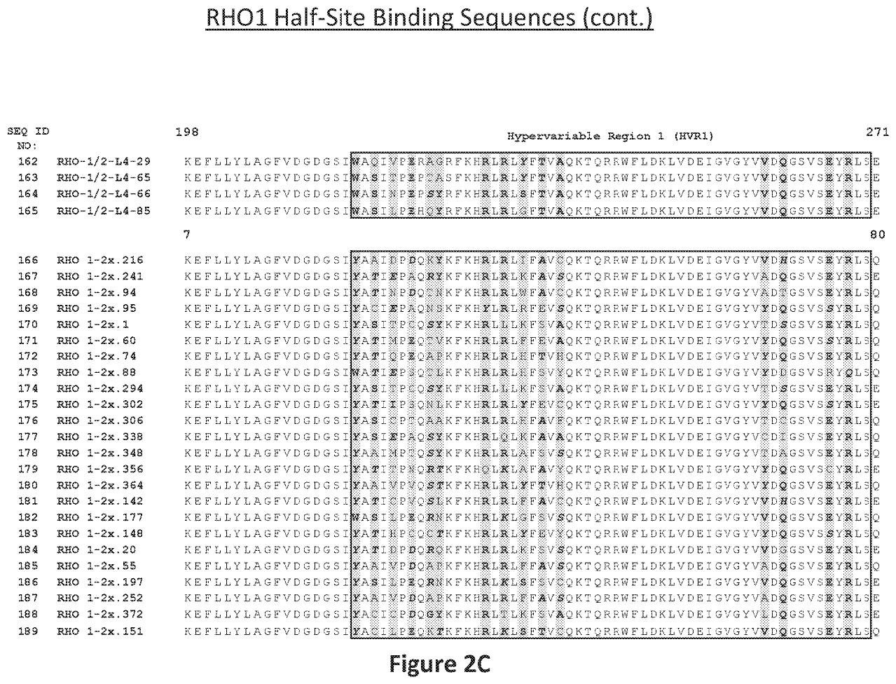

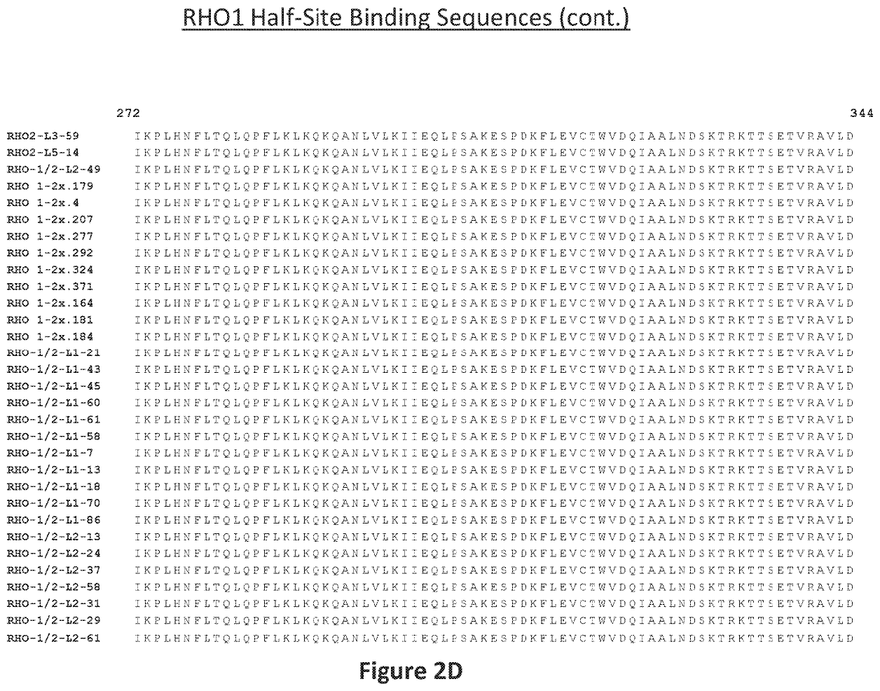

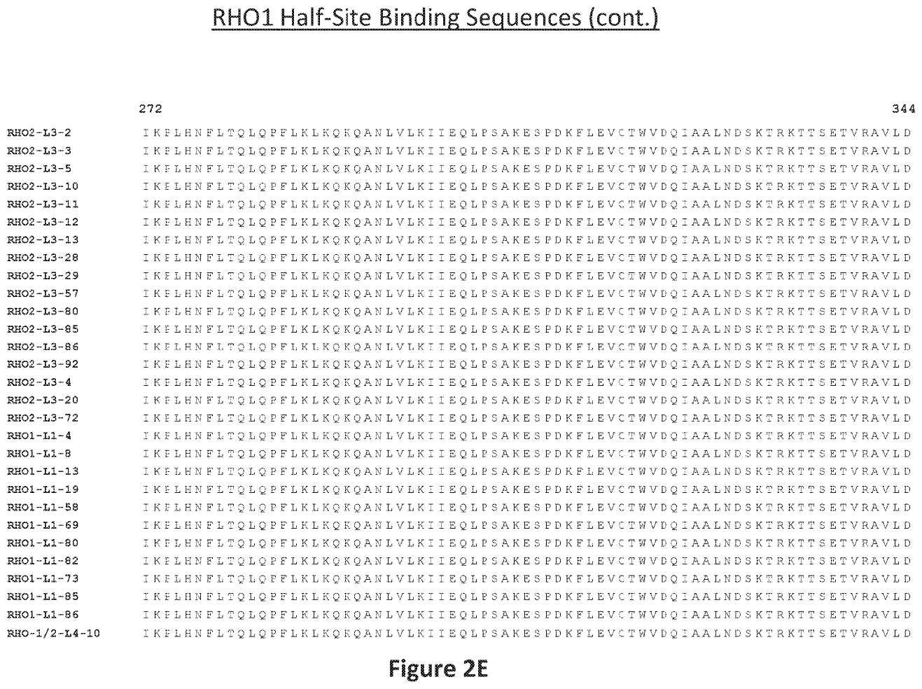

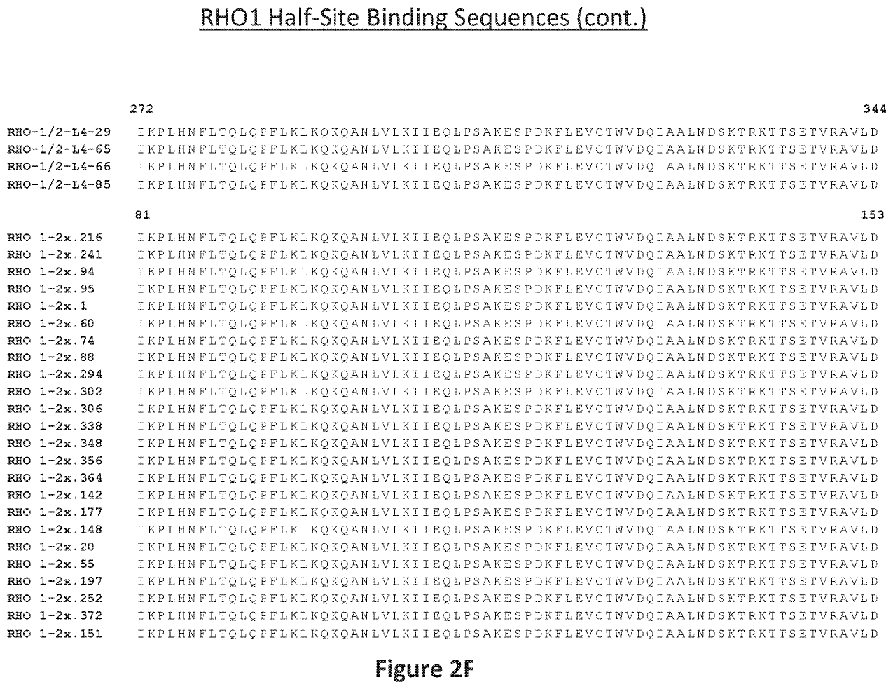

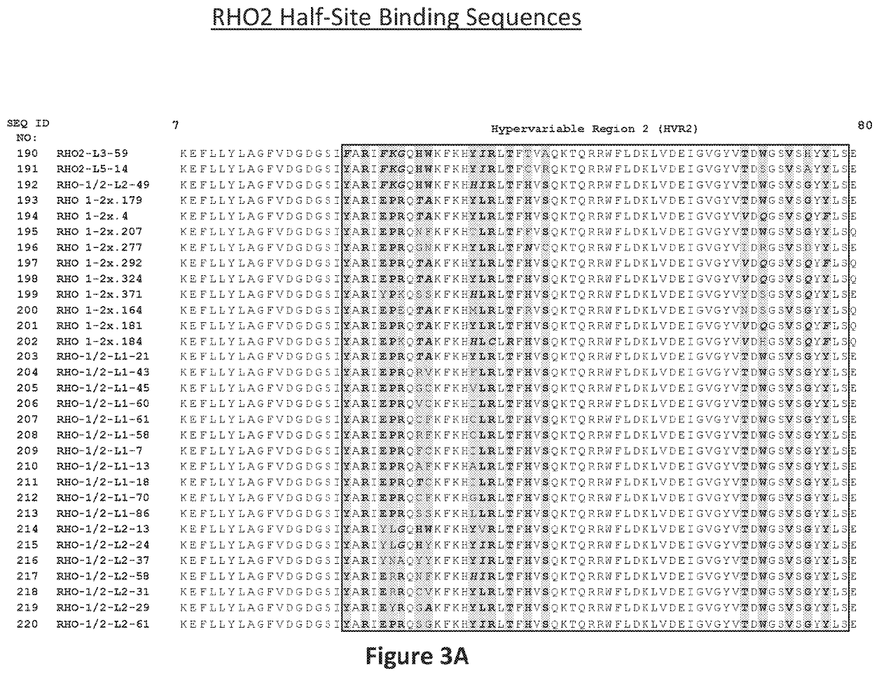

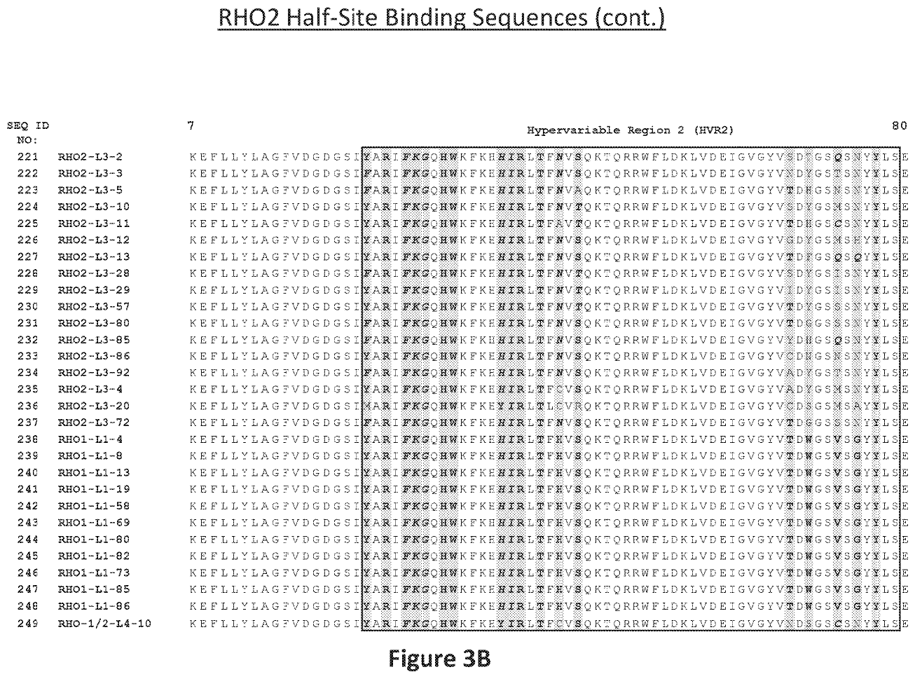

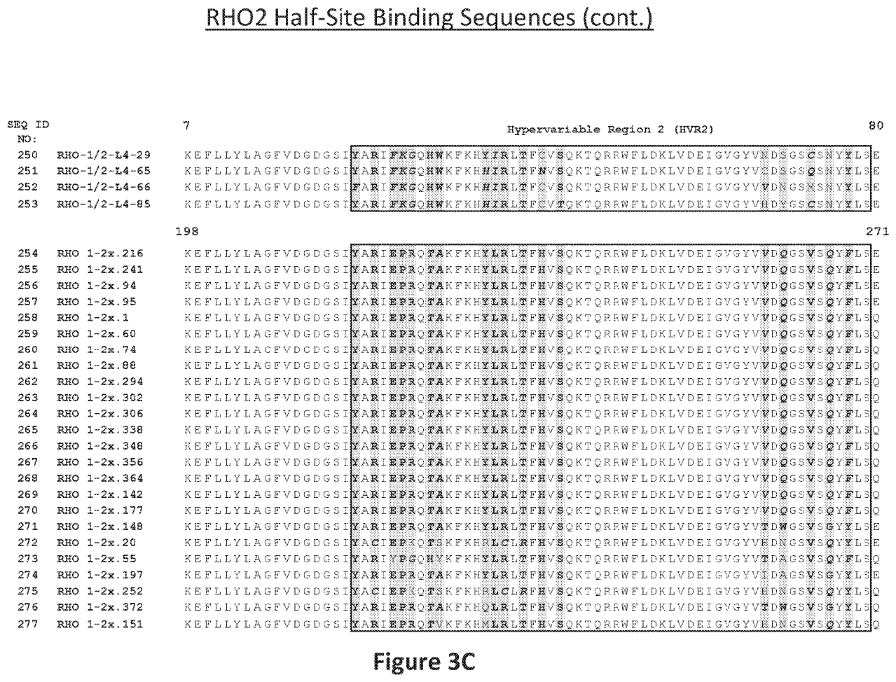

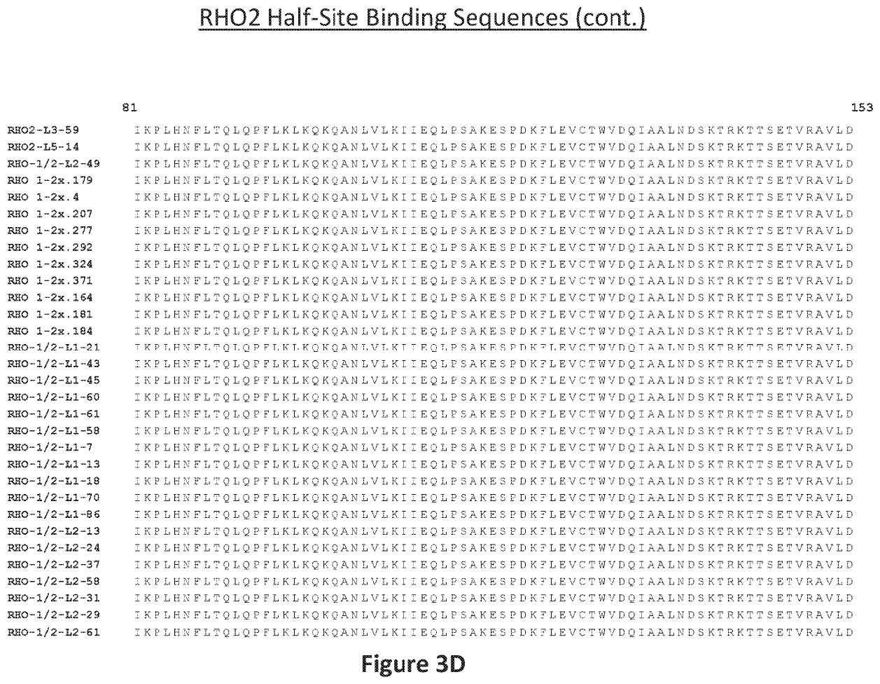

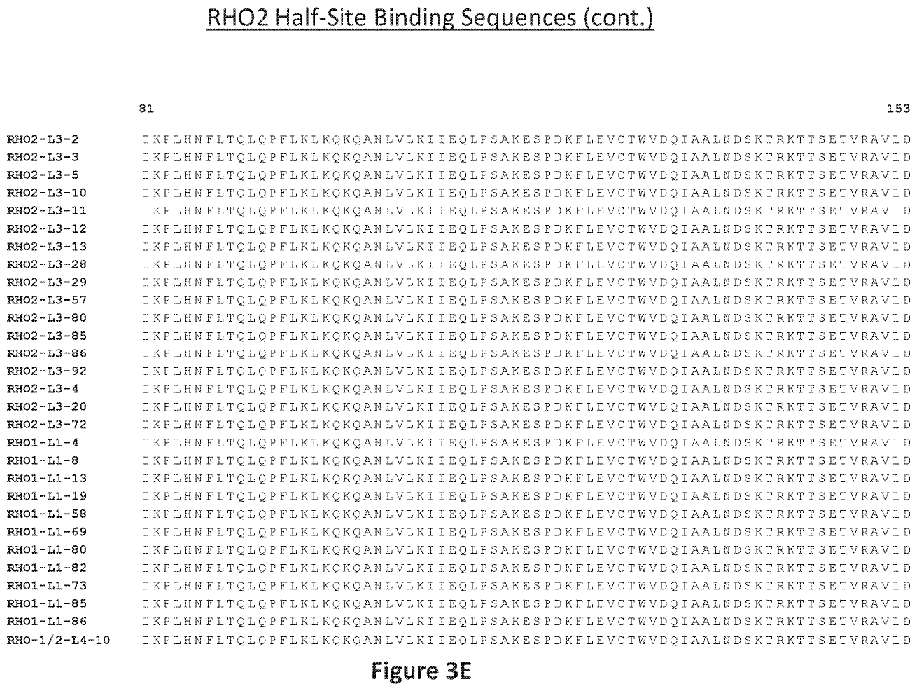

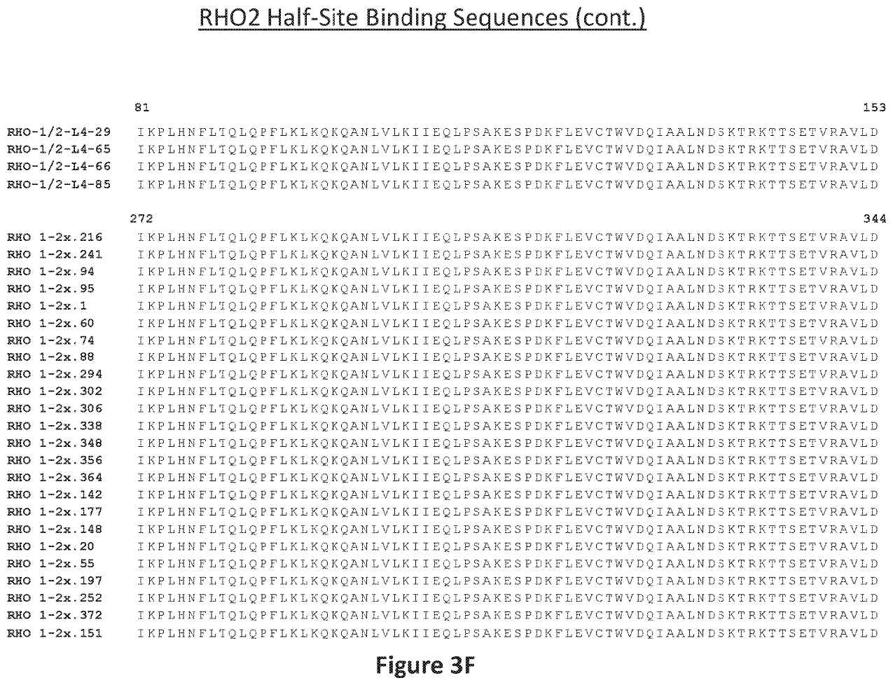

As illustrated in FIGS. 2 and 3, RHO1-binding subunits and RHO2-binding subunits each comprise a 56 base pair hypervariable region, referred to as HVRI and HVR2, respectively. RHO1-binding subunits are identical outside of the HVRI region except at position 80 or position 271 (comprising a Q or E residue), and are highly conserved within the HVRI region. Similarly, RHO2-binding subunits are also identical outside of the HVR2 region except at position 80 or position 271 (comprising a Q or E residue), and are highly conserved within the HVR2 region.

The RHO1-binding regions of SEQ ID NOs: 6-93 are illustrated in FIGS. 2A-2F and are provided as SEQ ID NOs: 102-189, respectively. Nearly all of SEQ ID NOs: 102-189 share at least 90% sequence identity to SEQ ID NO: 102, which is the RHO1-binding region of the meganuclease RHO2-L3-59 (SEQ ID NO:6). RHO2-binding regions of SEQ ID NOs: 6-93 are illustrated in FIGS. 3A-3F and are provided as SEQ ID NOs: 190-277, respectively. Nearly all of SEQ ID NOs: 190-277 share at least 90% sequence identity to SEQ ID NO: 190, which is the RHO2-binding region of the meganuclease RHO2-L3-59 (SEQ ID NO:6).

### 2. Cleavage of a P23H Recognition Sequence in a CHO Cell Reporter Assay

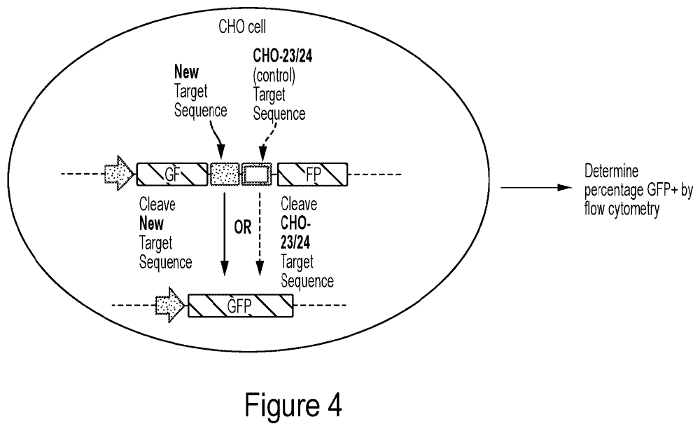

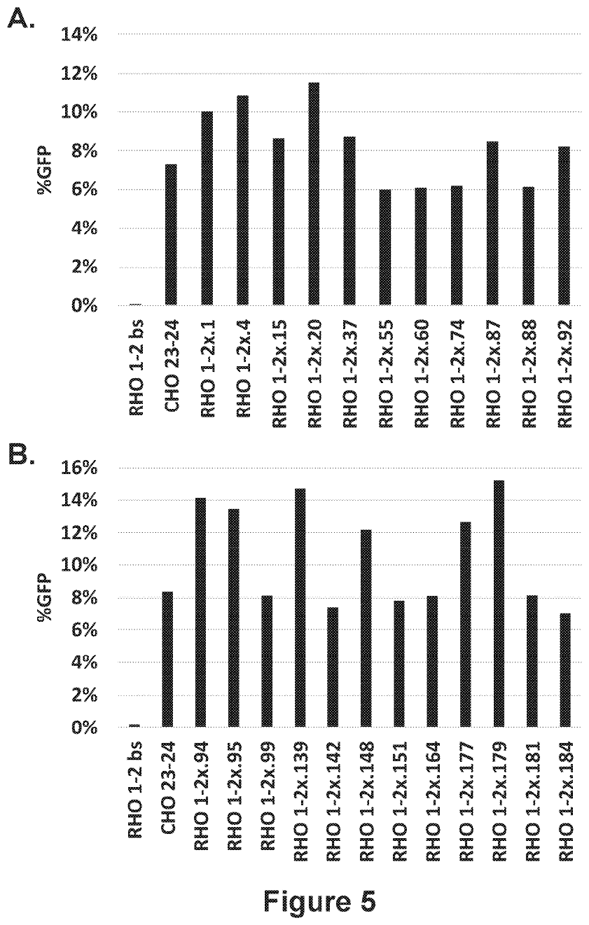

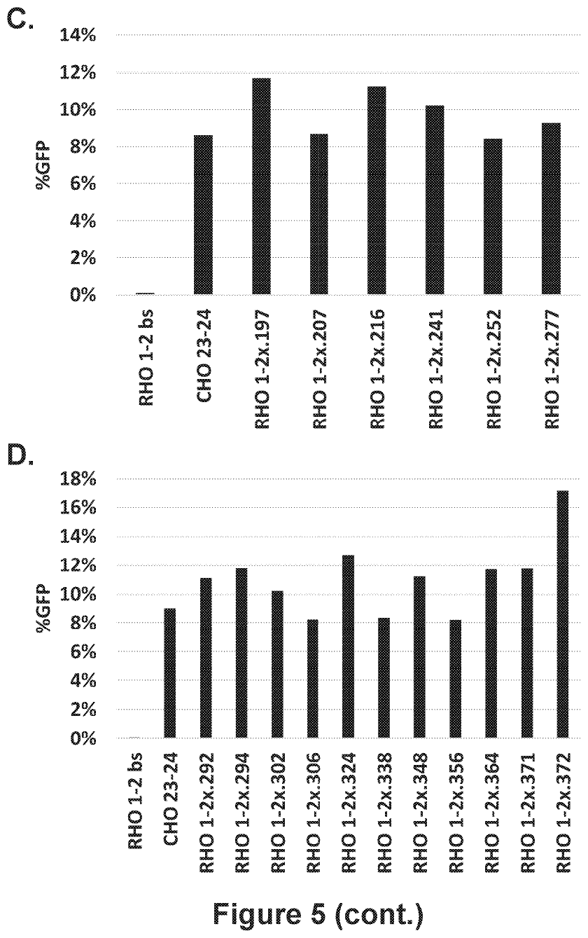

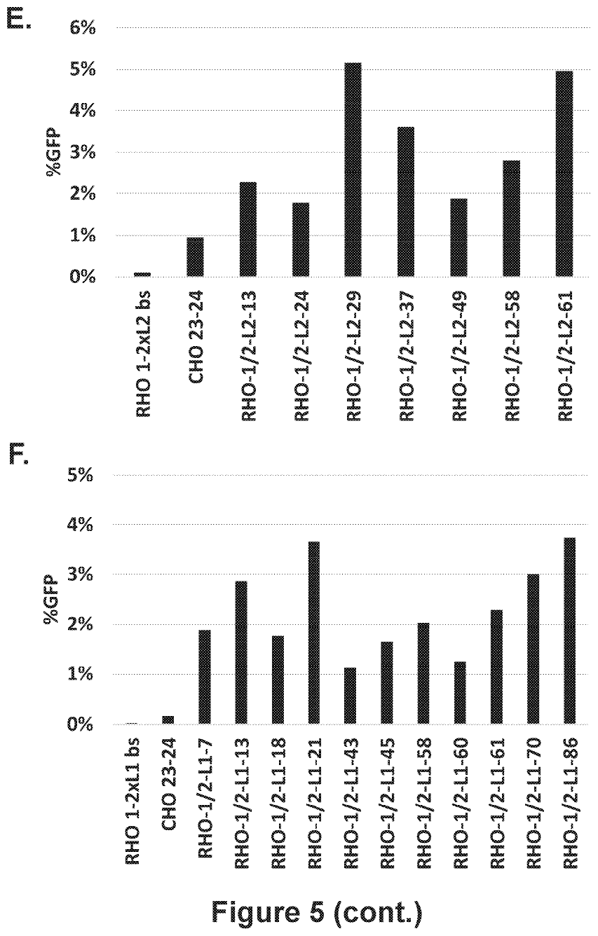

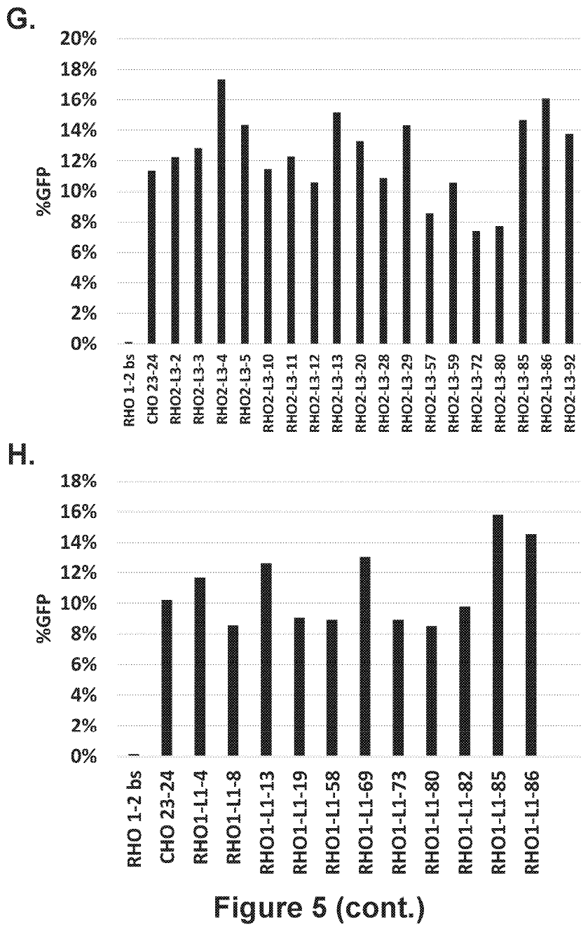

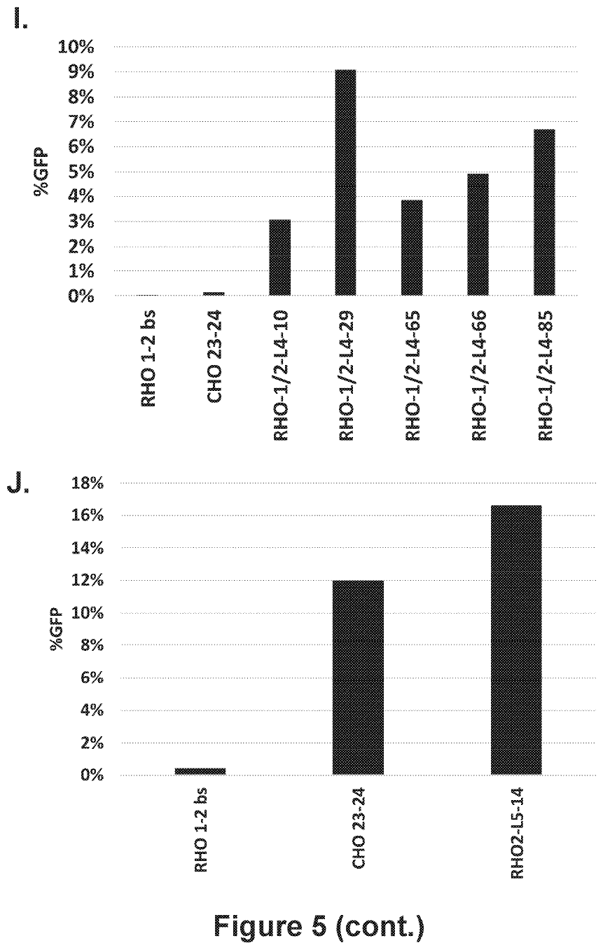

To determine whether RHO 1-2 meganucleases could recognize and cleave the P23H recognition sequence of SEQ ID NO:1, each RHO 1-2 meganuclease was evaluated using the CHO cell reporter assay previously described (see WO2012/167192, FIG. 4). To perform the assay, a pair of CHO cell reporter lines were produced which carried a non-functional Green Fluorescent Protein (GFP) gene expression cassette integrated into the genome of the cell. The GFP gene in each cell line was interrupted by a pair of recognition sequences such that intracellular cleavage of either recognition sequence by a meganuclease would stimulate a homologous recombination event resulting in a functional GFP gene. In both cell lines, one of the recognition sequences was derived from the RHO gene (either SEQ ID NO: 1 or SEQ ID NO: 5) and the second recognition sequence was specifically recognized by a control meganuclease called “CHO-23/24”. CHO reporter cells comprising the P23H recognition sequence of SEQ ID NO: 1 and the CHO-23/24 recognition sequence are referred to herein as “RHO 1-2 cells.” RHO 1-2 cells were transfected with plasmid DNA encoding one of the RHO 1-2 meganucleases (SEQ ID NOs: 6-93) or encoding the CHO-23/34 meganuclease. Approximately 4×105 CHO cells were transfected with 50 ng of plasmid DNA in a 96-well plate using Lipofectamine 2000 (ThermoFisher) according to the manufacturer's instructions. At 48 hours post-transfection, cells were evaluated by flow cytometry to determine the percentage of GFP-positive cells compared to an untransfected negative control (RHO 1-2 bs). All RHO 1-2 meganucleases were found to produce GFP-positive cells in cell lines comprising the P23H recognition sequence at frequencies significantly exceeding the negative control and comparable to or exceeding the CHO-23/24 positive control, indicating that each RHO 1-2 meganuclease was able to efficiently recognize and cleave the intended P23H recognition sequence in a cell (see, FIG. 5).

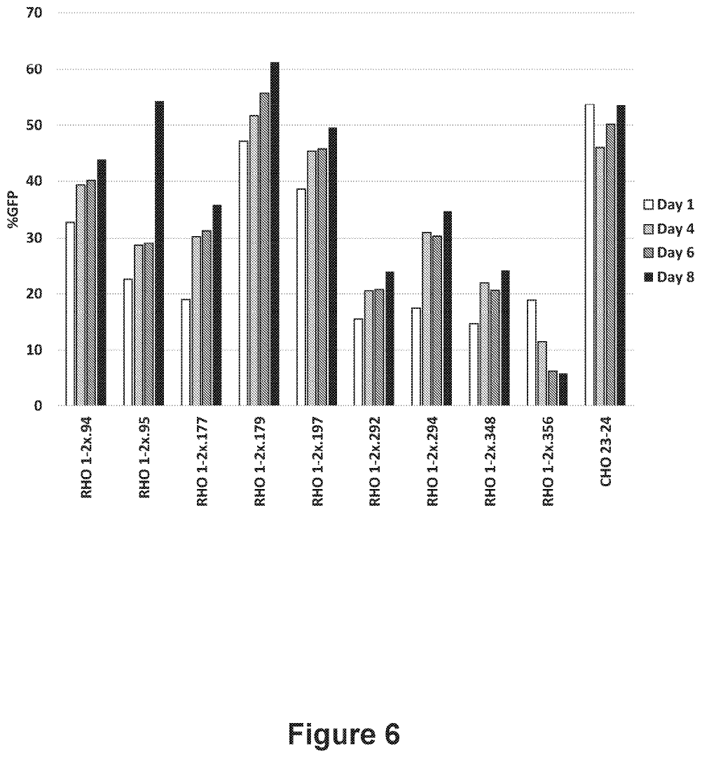

The efficacy of RHO 1-2 recombinant meganucleases was also determined in a time-dependent manner 1, 4, 6, and 8 days after introduction of the meganucleases into RHO 1-2 cells. In this study, RHO 1-2 cells (1.0×106) were electroporated with 1×106 copies of meganuclease mRNA per cell using a BioRad Gene Pulser Xcell according to the manufacturer's instructions. At 48 hours post-transfection, cells were evaluated by flow cytometry to determine the percentage of GFP-positive cells. A CHO-23/24 meganuclease was also included at each time point as a positive control. As shown in FIG. 6, the efficacy of RHO 1-2 meganucleases persisted over the 8 day period evaluated.

### 3. Conclusions

These studies demonstrated that RHO 1-2 meganucleases (SEQ ID NOs: 6-93) encompassed by the invention can target and cleave the P23H recognition sequence of SEQ ID NO: 1 in cells.

### Example 2

### Specificity of Meganucleases for a P23H Recognition Sequence

### 1. CHO Reporter Cells Comprising a Corresponding Wild-Type RHO Sequence

To determine the specificity of RHO 1-2 meganucleases (SEQ ID NOs: 6-93) for the P23H recognition sequence of SEQ ID NO:1, a CHO reporter cell line was generated as previously described that comprises the corresponding wild-type RHO recognition sequence (SEQ ID NO:5), referred to herein as the “RHO 3-4 recognition sequence,” and the CHO-23/24 recognition sequence. The resulting cells are referred to herein as “RHO 3-4 cells.”

### 2. Specificity of RHO 1-2 Meganucleases

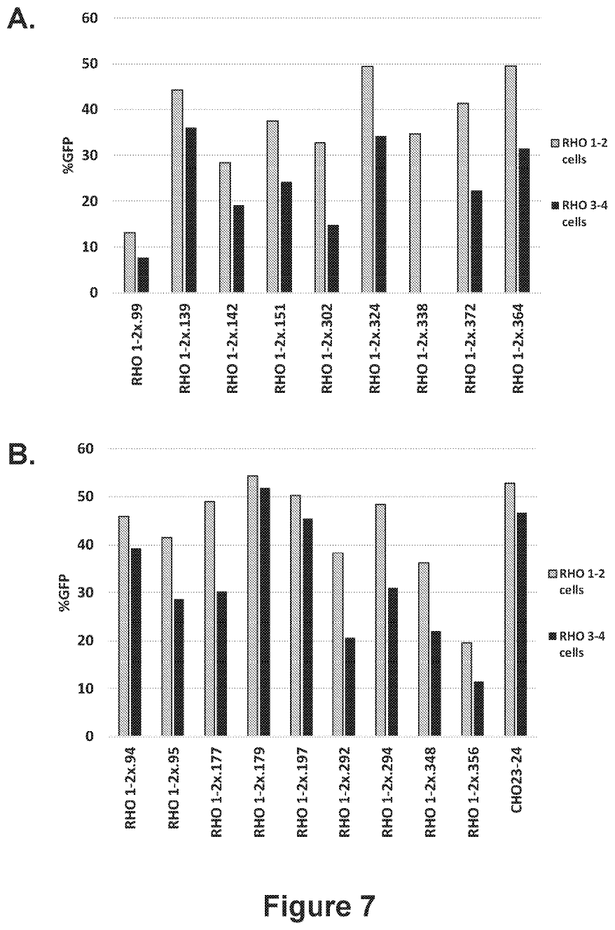

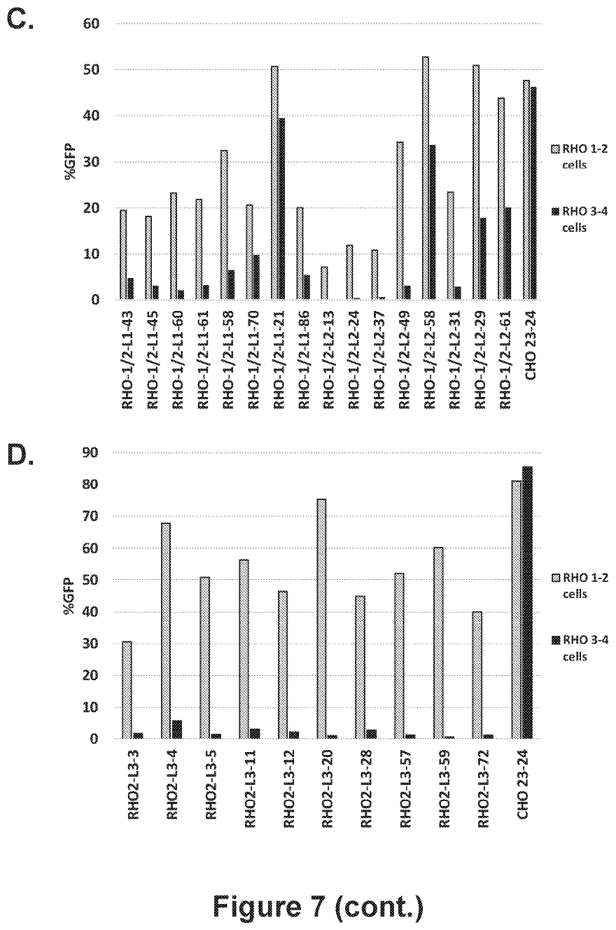

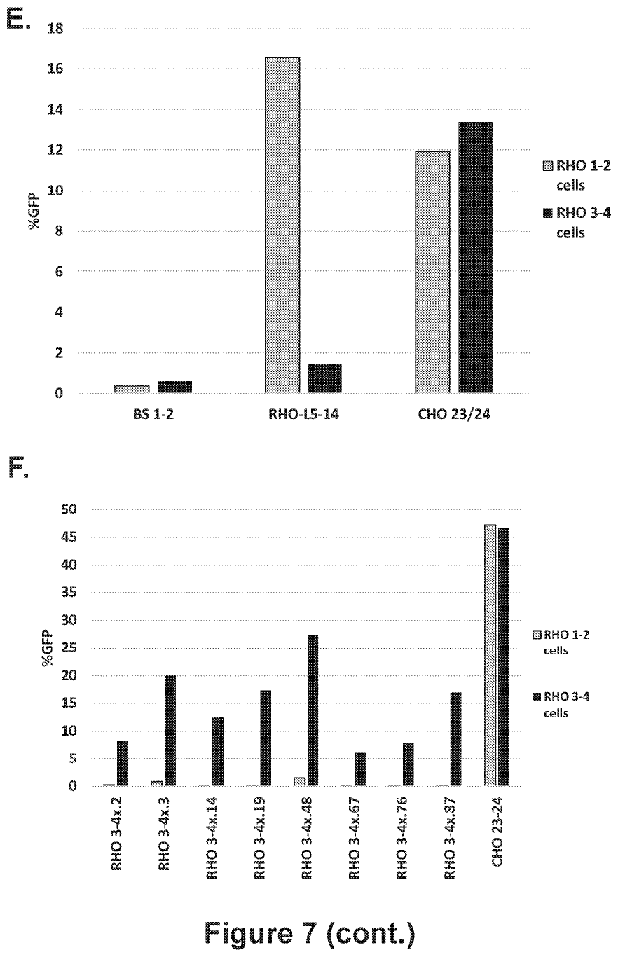

RHO 1-2 meganucleases were introduced into RHO 1-2 cells or RHO 3-4 cells to determine if the recombinant meganucleases could discriminate between the P23H recognition sequence of SEQ ID NO: 1 and the corresponding wild-type recognition sequence (SEQ ID NO: 5). RHO 1-2 meganuclease mRNA was introduced into RHO 1-2 cells or RHO 3-4 cells via electroporation of mRNA as previously described, and the percent GFP-positive cells was determined after 3-5 days. As shown in FIG. 7, each of the RHO 1-2 meganucleases tested preferentially cleaved the P23H recognition sequence relative to the RHO 3-4 recognition sequence, as evidenced by higher percentages of GFP-positive RHO 1-2 cells than RHO 3-4 cells (see, FIG. 7A-7E). In subsequent experiments, it was shown that recombinant meganucleases could be developed that preferentially target and cleave the wild-type RHO 3-4 recognition sequence (SEQ ID NO:5). This set of meganucleases, when evaluated using the CHO cell reporter assay described above, were found to yield a higher percentage of GFP-positive RHO 3-4 cells than RHO 1-2 cells (see, FIG. 7F).

### 3. Conclusions

We have demonstrated that RHO 1-2 meganucleases encompassed by the invention preferentially cleave the P23H recognition sequence of SEQ ID NO:1 relative to the corresponding wild-type RHO 3-4 recognition sequence (SEQ ID NO:5).

### Example 3

### Generation and Expression of Recombinant AAV Vectors for Expressing Recombinant Meganucleases

### 1. Recombinant AAV Vectors for Expressing RHO 1-2 Meganucleases

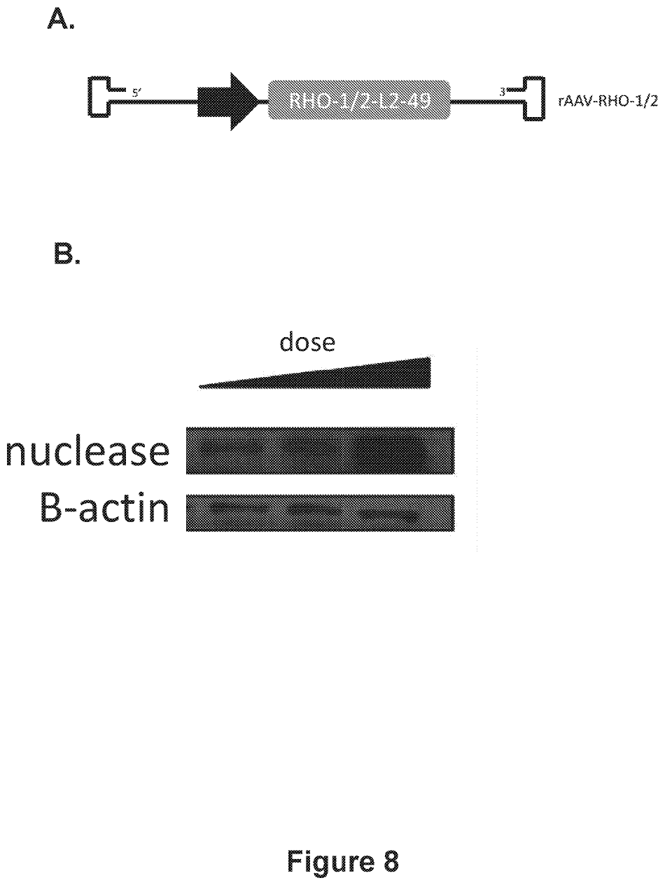

Recombinant AAV vectors were designed to express RHO 1-2 meganucleases in CHO reporter cells. As shown in FIG. 8A, the recombinant AAV vectors comprise, from 5′ to 3′, a first inverted terminal repeat (ITR), a CMV promoter operably linked to a nucleotide sequence encoding a meganuclease, and a second inverted terminal repeat. In this study, a coding sequence for the RHO-1/2-L2-49 meganuclease was incorporated into the recombinant AAV vector.

### 2. Expression of a RHO 1-2 Meganuclease Following Recombinant AAV Transduction

A recombinant AAV vector (rAAV-RHO-1/2) was prepared for expression of the RHO-1/2-L2-49 meganuclease in RHO 1-2 cells using a standard triple-transfection protocol in HEK-293 cells (Drittanti et al. (2001), J Gene Med. 3:59-71). RHO 1-2 CHO reporter cells were transduced with the rAAV-RHO-1/2 vector at three concentrations of virus. At 24 hours post-transduction, cells were lysed and analyzed by Western blot using a polyclonal meganuclease-specific antibody or a B-actin-specific control antibody. As shown in FIG. 8B, RHO-1/2-L2-49 expression was observed at all three concentrations of virus.

### 3. Specificity of AAV-Delivered RHO 1-2 Meganuclease

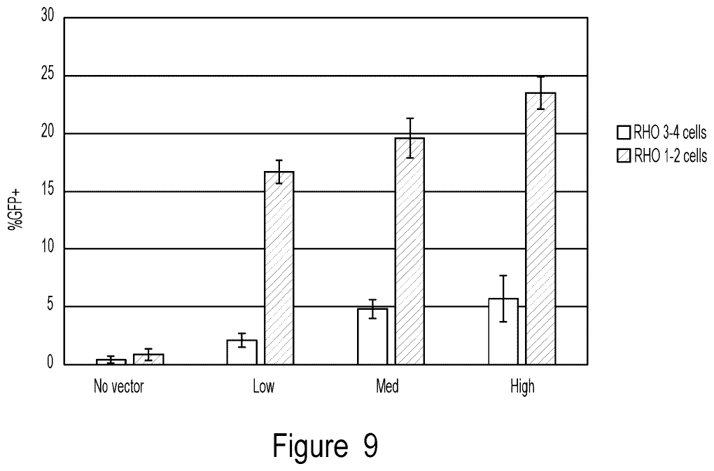

The rAAV-RHO-1/2 vector was transduced into RHO 1-2 cells or RHO 3-4 cells to demonstrate the specificity of AAV-delivered RHO-1/2-L2-49 meganuclease for recognizing and cleaving the P23H recognition sequence of SEQ ID NO: 1 relative to the corresponding wild-type RHO 3-4 recognition sequence (SEQ ID NO:5). As shown in FIG. 9, after three days post-transduction, the AAV-delivered RHO-1/2-L2-49 meganuclease induced a higher percentage of GFP-positive RHO 1-2 cells than RHO 3-4 cells at all concentrations of virus used, indicating that the meganuclease preferentially recognizes and cleaves the mutant sequence.

### 4. Persistence of AAV-Delivered RHO 1-2 Meganuclease Expression

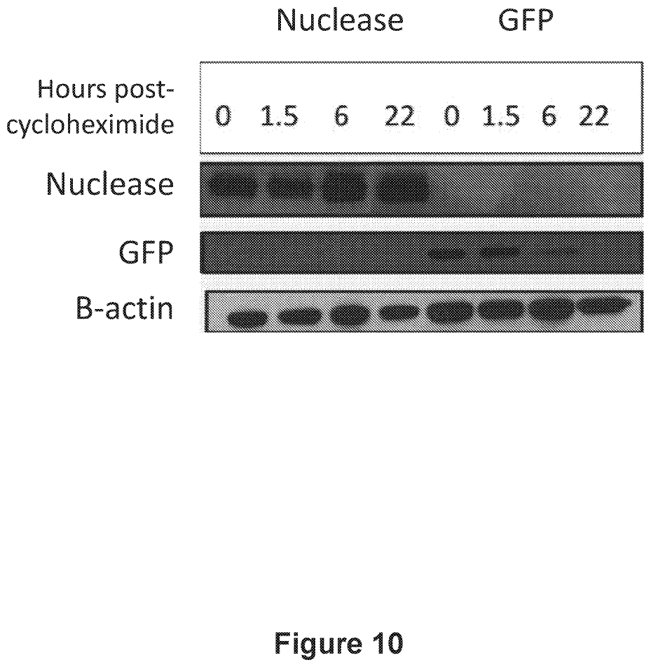

RHO 1-2 cells, transduced with the recombinant AAV vector encoding the RHO-1/2-L2-49 meganuclease or GFP, were treated with cycloheximide to halt protein translation and determine the stability of the expressed proteins in cells. Cells were lysed at various times (0, 1.5, 6, and 22 hours) post-cycloheximide treatment, and protein levels of RHO-1/2-L2-49, GFP, and B-actin were determined by Western Blot analysis. As shown in FIG. 10, RHO-1/2-L2-49 protein persisted significantly longer than GFP, with no reduction in meganuclease protein apparent after 22 hours.

### 5. Conclusions

This study indicates that RHO 1-2 meganucleases encompassed by the invention can be expressed in cells using recombinant AAV vectors, and that such meganucleases can preferentially recognize and cleave the P23H recognition sequence of SEQ ID NO: 1 relative to the corresponding wild-type RHO 3-4 sequence (SEQ ID NO:5). Furthermore, this study demonstrates that RHO 1-2 meganuclease proteins are stable in cells when expressed using recombinant AAV vectors.

### Example 4

### Cleavage of RHO 1-2 Recognition Sequence in Reporter Cells with RHO 1-2 Meganucleases Delivered by AAV

### 1. Production of Recombinant AAV Vectors

The purpose of this study was to demonstrate that RHO 1-2 meganucleases could be expressed in mammalian cells by viral transduction, and to further demonstrate their ability to cleave the RHO 1-2 recognition sequence.

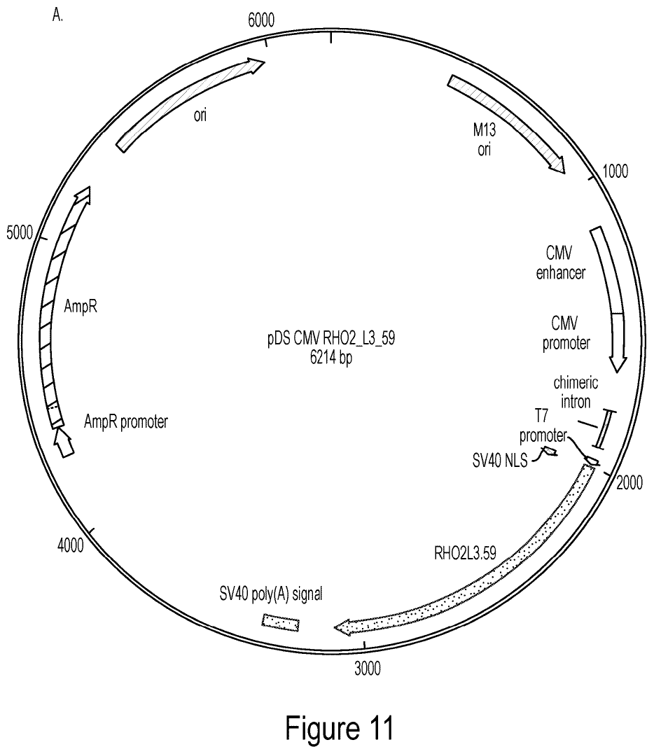

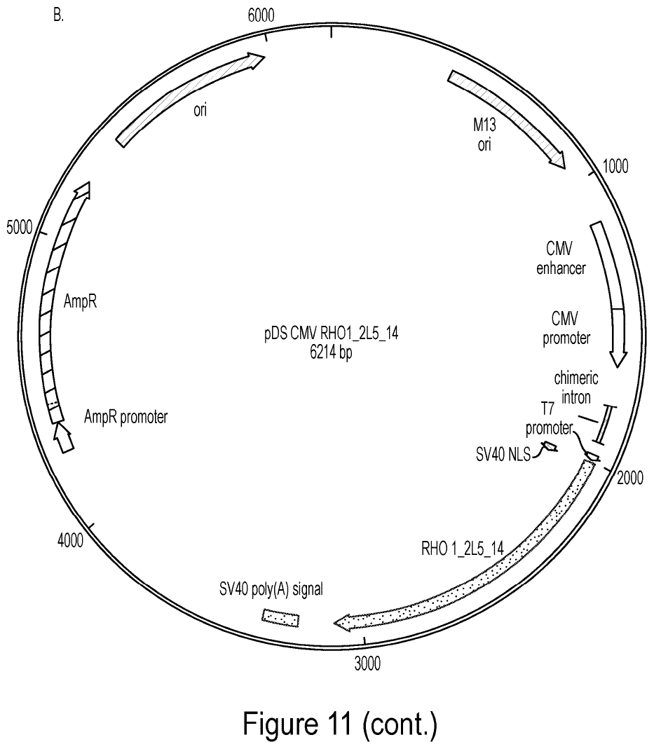

For this experiment, two recombinant AAV2 vectors were produced by the triple-transfection method previously described. The two donor plasmids were pDS CMV RHO2_L3_59 (SEQ ID NO:248) and pDS CMVRHO2_L5_14 (SEQ ID NO:249), which encoded the RHO2-L3-59 and RHO2-L5-14 meganucleases, respectively (FIG. 11). These plasmids comprise a CMV promoter and enhancer driving expression of the nuclease, an SV40 polyA sequence, AAV ITR (inverted terminal repeat) sequences required for genome replication, and are suitable for the production of self-complimentary AAV genomes (scAAV). The capsid from AAV serotype 2 was used to generate these vectors. Recombinant scAAV was produced using the triple-transfection method in HEK 293 cells and purified over a CsC1 gradient. Viral titer was determined by RT-PCR and slot-blot methods.

### 2. Transduction of Reporter Cells and Analysis of Cleavage Efficiency

To determine whether these AAV particles could deliver functional RHO 1-2 meganucleases capable of recognizing and specifically cleaving the RHO 1-2 recognition sequence (SEQ ID NO:1) and not the RHO 3-4 recognition sequence (SEQ ID NO:5), they were tested in previously described CHO reporter lines (see WO2012/167192). To perform the assay, a pair of CHO cell reporter lines were produced which carried a non-functional Green Fluorescent Protein (GFP) gene expression cassette integrated into the genome of the cell. The GFP gene in each cell line was interrupted by either the RHO 1-2 or RHO 3-4 recognition sequence such that intracellular cleavage of the recognition sequence by a meganuclease would stimulate a homologous recombination event resulting in a functional GFP gene. CHO reporter cells comprising the RHO 1-2 recognition sequence are referred to in this experiment as “P23H RHO cells.” CHO reporter cells comprising the RHO 3-4 recognition sequence are referred to in this experiment as “WT RHO cells.” Wild-type control cells that do not harbor the GFP reporter cassette are referred to in this experiment as “CHO K cells.”

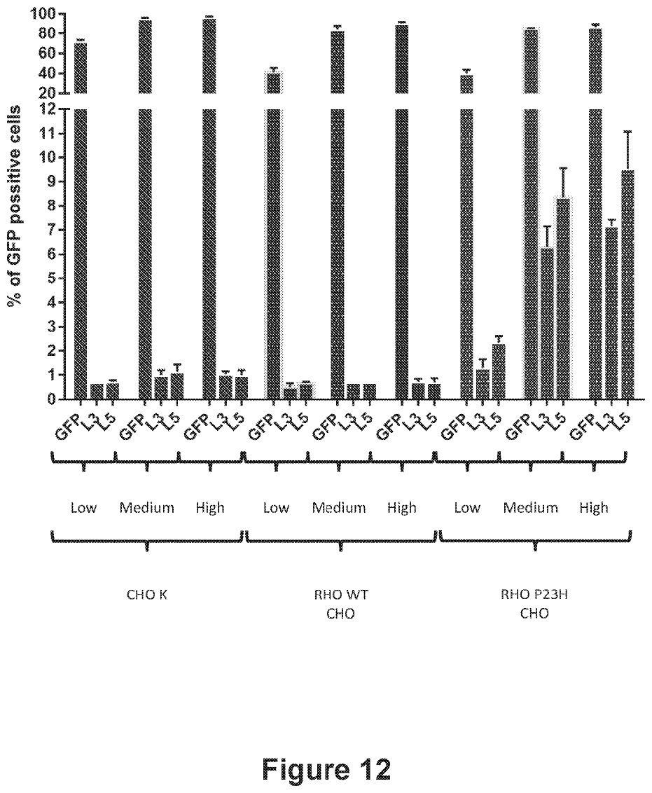

WT RHO cells, P23H RHO cells, or wild-type CHO K cells (negative control) were infected with recombinant scAAV described above. Reporter cells were infected at three MOIs: 1×108, 1×109 or 2×109 (“low,” “medium,” and “high,” respectively). At 48 hours post-infection, cells were evaluated by flow cytometry to determine the percentage of GFP-positive cells compared to wild-type control CHO cells. As shown in FIG. 12, control wild-type CHO cells infected with either the RHO2-L3-59 or RHO2-L5-14 scAAV provided background GFP expression, less than 1%. Cells harboring the wild-type RHO 3-4 target (“WT RHO cells”) did not display GFP expression significantly higher than the control cells, indicating that the RHO 1-2 meganucleases are not capable of cleaving the RHO 3-4 recognition sequence. In P23H RHO cells, both the RHO2-L3-59 and RHO2-L5-14 meganucleases resulted in significant GFP expression in a dose-dependent manner, with levels approaching 10% GFP+ in the highest dose.

Western blot analysis was used to confirm both GFP and meganuclease expression in the P23H RHO cells. Whole cell lysates were prepared only from P23H RHO and WT RHO cells infected with the three different doses of scAAV. Lysates for duplicates were harvested to provide a measure of reproducibility. Equivalent amounts of lysates (determined by protein concentration) were resolved by SDS-PAGE, transferred to a membrane and probed using antibodies against either GFP, the I-CreI meganuclease, or for loading control, B-actin. RHO 1-2 meganucleases are detectable with the polyclonal antibody against 1-CreI.

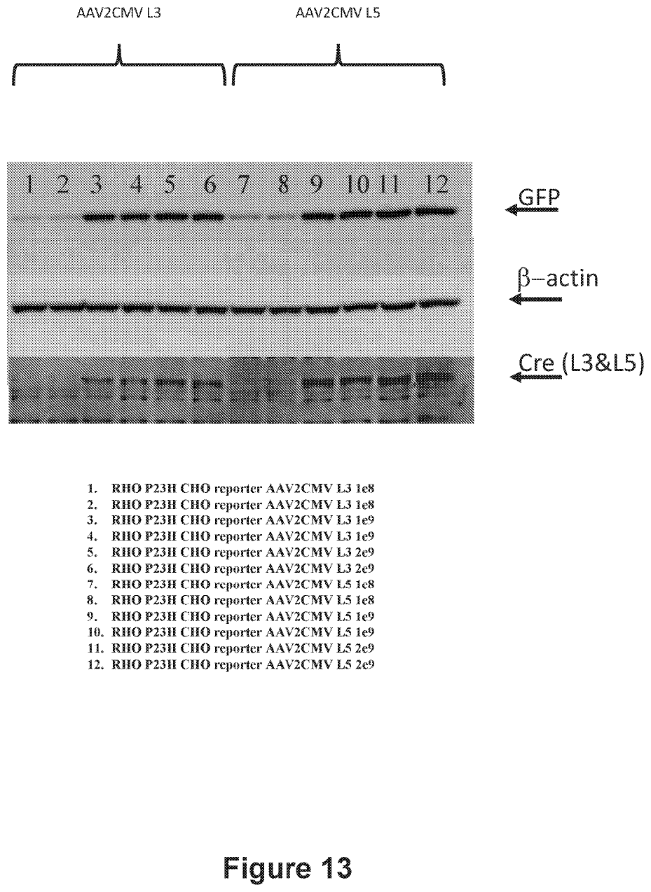

As shown in FIG. 13, western blot analysis clearly demonstrates detection of GFP in a dose-dependent manner, confirming the flow cytometry data shown in FIG. 12 and described above. Western blot analysis also shows expression of the RHO 1-2 meganucleases, also in a dose-dependent manner. The blot does suggest that RHO2-L5-14 expressed to higher levels than RHO2-L3-59. B-actin levels were consistent, indicating proper gel loading.

### 3. Conclusions

Together, these data demonstrate recombinant scAAV carrying expression cassettes for RHO 1-2 meganucleases of the invention are able to infect CHO reporter lines, resulting in expression of the RHO 1-2 meganucleases which are then able to specifically cleave the P23H RHO 1-2 recognition sequence (SEQ ID NO:1) and not the WT RHO 3-4 recognition.

### Example 5

### In Vivo Cleavage of P23H Allele in Mouse Model of RP

### 1. Production of Recombinant AAV Vectors and Sub-Retinal Delivery to Mouse Eve

The purpose of this study was to determine whether RHO 1-2 meganucleases of the invention could target and cleave the RHO 1-2 recognition sequence in vivo within photoreceptor cells of a mouse retina.

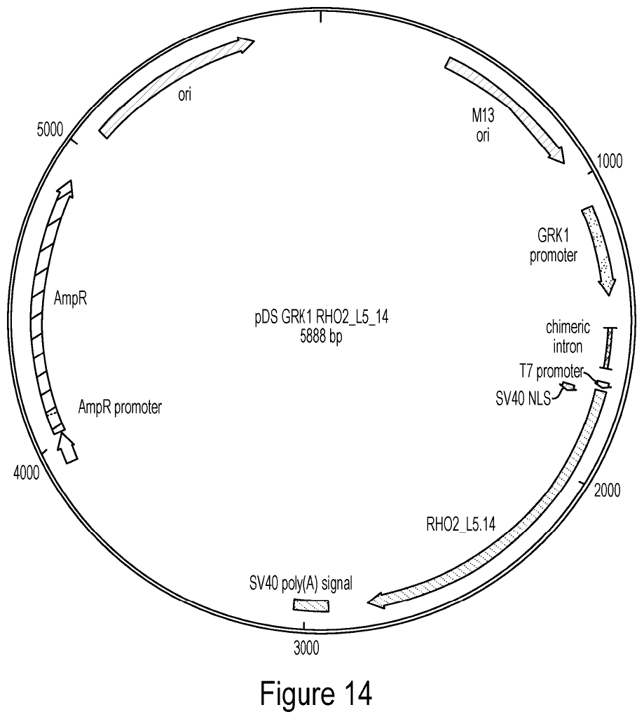

Recombinant scAAV were prepared using the triple-transfection method as described above and were tested in a murine model of retinitis pigmentosa. In this model, transgenic mice carry a single copy of the human P23H mutant RHO gene in addition to the endogenous murine RHO alleles. While these mice do not exhibit a retinitis pigmentosa phenotype, they are useful for molecular analysis of in vivo cleavage of the RHO 1-2 recognition sequence. Since RHO2-L5-14 performed better in the GFP reporter CHO lines, only scAAV encoding RHO2-L5-14 was used to infect mice (though the present experiments could be performed using the RHO2-L3-59 meganuclease, including the production of an scAAV using the donor plasmid set forth in SEQ ID NO: 280). The pDS GRK1 RHO2_L5_14 donor plasmid (SEQ ID NO: 281) used in the scAAV triple transfection protocol is illustrated in FIG. 14. As shown, the RHO2-L5-14 meganuclease was under the control of a rod cell-specific GRKI promoter. As a control, a recombinant scAAV encoding a GFP expression cassette was also prepared. The capsid from AAV serotype 5 was used to generate these recombinant scAAV vectors.

One of the P23H transgenic mice was used to test whether sub-retinal injection of an AAV encoding a RHO2-LS-14 expression cassette could result in cleavage at the RHO 1-2 recognition sequence. Briefly, at postnatal day 30, a mouse was placed under general anesthesia by intraperitoneal injection of Ketamine 100 mg/kg and Xylazine 10 mg/kg. Pupils were dilated with Tropicamide (0.5%) and 1% Proparacaine. Under an ophthalmic surgery microscope, with a 30-gauge needle a small incision was made through the cornea adjacent to the limbus. A 33-gauge blunt needle fitted to a Hamilton syringe was inserted through the incision. All injections were made sub-retinally within the nasal quadrant of the retina. The mouse received 1 μL of scAAV encoding RHO2-L5-14 in one eye and 1 μL of scAAV encoding GFP in the other eye (both viral preparations were at a concentration of 7×1012 particles/mL). Visualization during injection was aided by the addition of fluorescein to the vector suspensions. Fundus and OCT examination were performed to confirm the successful sub-retinal delivery.

The mouse was euthanized with isoflurane and eyes were enucleated at 30 days post-injection. The retinas were carefully dissected free from the other ocular tissues under surgical microscope. DNA was isolated from dissected retinas by using DNA isolation kit (Qiagen). Briefly, retinas were digested with lysis buffer containing protease Kat 55° C. for 2 hrs. After lysis, crude extract was passed through the column and bound DNA washed several times. The DNA was eluted and concentration was estimated by NanoDrop (Thermo).

To determine the presence and relative frequency of mutations at the RHO 1-2 recognition sequence, the RHO 1-2 locus was PCR-amplified and subjected to deep sequencing analysis. Briefly, PCR primers were designed to amplify a ˜200 bp region spanning the RHO 1-2 recognition sequence and gel-purified PCR bands were subjected to deep sequencing analysis using an Illumina MiSeq instrument.

### 2. Results

Deep sequencing of the PCR bands provided over 5×10⁵ sequences per sample. Retinas injected with scAAV encoding GFP showed about 4×10³ sequences with indels (insertions or deletions) at the RHO 1-2 recognition sequence, establishing the background at 0.54%. In DNA isolated from a mouse retina that had been injected with AAV encoding the RHO2-L5-14 meganuclease, indels were detected at 3.92%, approximately 7-fold higher than background (Table 3). In other experiments (data not shown), control mice showed a lower frequency of indels, typically around 0.01%.

TABLE 3

| | Sequences with | Sequences with | |
|---|---|---|---|
| | indels | no indels | % Indel |
| AAV-GFP | 4155 | 766145 | 0.54 |
| AAV-RHO 1-2L5.14 | 22330 | 547763 | 3.92 |

### 3. Conclusions

The deep sequencing data demonstrates that sub-retinal injection with an AAV encoding the RHO2-L5-14 meganuclease resulted in mutation of the P23H RHO 1-2 recognition sequence in a murine model of retinitis pigmentosa following cleavage and non-homologous end joining.

### Example 6

### In Vivo Expression of RHO 1-2 Meganucleases in Retinal Cells

### 1. Western Blot Analysis of Retinal Cells

To confirm that the RHO2-L5-14 meganuclease could be expressed in the retinal cells of wild-type mice following AAV delivery, five wild-type mice were administered scAAV encoding RHO2-L5-14 by sub-retinal injection as described above. Both the OS (left) and OD (right) eyes were infected. 30 days following injection, retinas were dissected, whole cell lysates were prepared and western blot analysis was performed, using the anti-I-CreI antibody as described above.

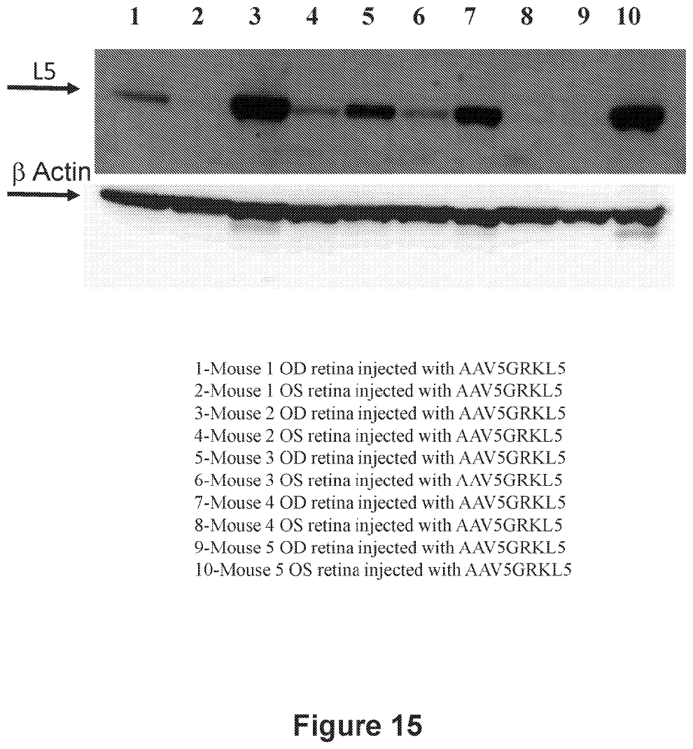

Western blot analysis showed that in most retinas, RHO2L-5-14 expression is readily detected (FIG. 15, lanes 3, 5, 7 and 10). In other retinas, expression was considerably less, but still detectable (FIG. 15, lanes 1, 4, and 6). The remaining retinas had almost undetectable levels of expression (FIG. 15, lanes 2, 8 and 9). Sub-retinal injection relies on precise delivery to a very small area behind the eye. Although this procedure is fairly common in adult patients, it is not a common lab practice in mouse models. Therefore, the difference in expression was attributed to the difficulty of murine sub-retinal injections.

### 2. Conclusions

Western blot analysis demonstrated that scAAV encoding RHO2L-5-14 delivered sub-retinally to mice resulted in expression of the RHO 1-2 meganuclease. Taken together with indel data from a murine model for the human P23H RHO gene, these data suggest that AAV delivery of a RHO 1-2 meganuclease is effective in causing deletions in the P23H RHO allele.

1. A recombinant meganuclease that recognizes and cleaves a P23H recognition sequence, wherein said recombinant meganuclease comprises a first subunit and a second subunit, wherein said first subunit recognizes a first recognition half-site of said P23H recognition sequence and comprises:
(a) an amino acid sequence having at least 80% sequence identity to residues 198-344 of any one of SEQ ID NOs: 6-69 or residues 7-153 of any one of SEQ ID NOs: 70-93; and
(b) a first hypervariable (HVR1) region;
and wherein said second subunit recognizes a second recognition half-site of said P23H recognition sequence and comprises:
(i) an amino acid sequence having at least 80% sequence identity to residues 7-153 of any one of SEQ ID NOs: 6-69 or residues 198-344 of any one of SEQ ID NOs: 70-93; and
(ii) a second hypervariable (HVR2) region.

2. The recombinant meganuclease of claim 1, wherein said first subunit comprises an amino acid sequence having at least 85% sequence identity to residues 198-344 of any one of SEQ ID NOs: 6-69 or residues 7-153 of any one of SEQ ID NOs: 70-93, and wherein said second subunit comprises an amino acid sequence having at least 85% sequence identity to residues 7-153 of any one of SEQ ID NOs: 6-69 or residues 198-344 of any one of SEQ ID NOs: 70-93.

3. The recombinant meganuclease of claim 1 or claim 2, wherein said first subunit comprises an amino acid sequence having at least 90% sequence identity to residues 198-344 of any one of SEQ ID NOs: 6-69 or residues 7-153 of any one of SEQ ID NOs: 70-93, and wherein said second subunit comprises an amino acid sequence having at least 90% sequence identity to residues 7-153 of any one of SEQ ID NOs: 6-69 or residues 198-344 of any one of SEQ ID NOs: 70-93.

4. The recombinant meganuclease of any one of claims 1-3, wherein said first subunit comprises an amino acid sequence having at least 95% sequence identity to residues 198-344 of any one of SEQ ID NOs: 6-69 or residues 7-153 of any one of SEQ ID NOs: 70-93, and wherein said second subunit comprises an amino acid sequence having at least 95% sequence identity to residues 7-153 of any one of SEQ ID NOs: 6-69 or residues 198-344 of any one of SEQ ID NOs: 70-93.

5. The recombinant meganuclease of any one of claims 1-4, wherein said P23H recognition sequence comprises SEQ ID NO:1 and said HVR1 region comprises W or Y at a position corresponding to:
(A) position 215 of any one of SEQ ID NOs: 6-69; or
(B) position 24 of any one of SEQ ID NOs: 70-93.

6. The recombinant meganuclease of any one of claims 1-5, wherein said P23H recognition sequence comprises SEQ ID NO: 1 and said HVR1 region comprises residues 215-270 of any one of SEQ ID NOs: 6-69 or residues 24-79 of any one of SEQ ID NOs: 70-93.

7. The recombinant meganuclease of any one of claims 1-6, wherein said P23H recognition sequence comprises SEQ ID NO: 1 and said HVR2 region comprises residues 24-79 of any one of SEQ ID NOs: 6-69 or residues 215-270 of any one of SEQ ID NOs: 70-93.

8. The recombinant meganuclease of any one of claims 1-7, wherein said recombinant meganuclease is a single-chain meganuclease comprising a linker, wherein said linker covalently joins said first subunit and said second subunit.

9. The recombinant meganuclease of any one of claims 1-8, wherein said first subunit comprises residues 198-344 of any one of SEQ ID NOs: 6-69 or residues 7-153 of any one of SEQ ID NOs: 70-93.

10. The recombinant meganuclease of any one of claims 1-9, wherein said second subunit comprises residues 7-153 of any one of SEQ ID NOs: 6-69 or residues 198-344 of any one of SEQ ID NOs: 70-93.

11. The recombinant meganuclease of any one of claims 1-10, wherein said recombinant meganuclease comprises the amino acid sequence of any one of SEQ ID NOs: 6-93.

12. The recombinant meganuclease of any one of claims 1-11, wherein said P23H recognition sequence comprises one of SEQ ID NOs: 1-4.

13. The recombinant meganuclease of any one of claims 1-12, wherein said P23H recognition sequence comprises SEQ ID NO: 1.

14. The recombinant meganuclease of any one of claims 1-13, wherein said recombinant meganuclease preferentially recognizes and cleaves a P23H recognition sequence comprising SEQ ID NO: 1 relative to a recognition sequence comprising SEQ ID NO: 5.

15. An isolated polynucleotide comprising a nucleic acid sequence encoding said recombinant meganuclease of any one of claims 1-14.

16. A recombinant DNA construct comprising said isolated polynucleotide of claim 15.

17. The recombinant DNA construct of claim 16, wherein said recombinant DNA construct encodes a recombinant adeno-associated virus (AAV) vector.

18. A recombinant AAV vector comprising said isolated polynucleotide of claim 15.

19. A method for treating retinitis pigmentosa in a subject in need thereof, said method comprising contacting the DNA of a target cell of said subject with a recombinant meganuclease that recognizes and cleaves a P23H recognition sequence, wherein said target cell comprises said P23H recognition sequence in a RHO gene allele, and wherein cleavage of said P23H recognition sequence inhibits expression of said RHO gene allele, wherein said recombinant meganuclease comprises a first subunit and a second subunit, wherein said first subunit recognizes a first recognition half-site of said P23H recognition sequence and comprises:
(a) an amino acid sequence having at least 80% sequence identity to residues 198-344 of any one of SEQ ID NOs: 6-69 or residues 7-153 of any one of SEQ ID NOs: 70-93; and
(b) a first hypervariable (HVR1) region;
and wherein said second subunit recognizes a second recognition half-site of said P23H recognition sequence and comprises:
(i) an amino acid sequence having at least 80% sequence identity to residues 7-153 of any one of SEQ ID NOs: 6-69 or residues 198-344 of any one of SEQ ID NOs: 70-93; and
(ii) a second hypervariable (HVR2) region.

20. The method of claim 19, wherein said method is for treating autosomal dominant retinitis pigmentosa.

21. The method of claim 19 or claim 20, wherein said first subunit comprises an amino acid sequence having at least 85% sequence identity residues 198-344 of any one of SEQ ID NOs: 6-69 or residues 7-153 of any one of SEQ ID NOs: 70-93, and wherein said second subunit comprises an amino acid sequence having at least 85% sequence identity to residues 7-153 of any one of SEQ ID NOs: 6-69 or residues 198-344 of any one of SEQ ID NOs: 70-93.

22. The method of any one of claims 19-21, wherein said first subunit comprises an amino acid sequence having at least 90% sequence identity to residues 198-344 of any one of SEQ ID NOs: 6-69 or residues 7-153 of any one of SEQ ID NOs: 70-93, and wherein said second subunit comprises an amino acid sequence having at least 90% sequence identity to residues 7-153 of any one of SEQ ID NOs: 6-69 or residues 198-344 of any one of SEQ ID NOS: 70-93.

23. The method of any one of claims 19-22, wherein said first subunit comprises an amino acid sequence having at least 95% sequence identity to residues 198-344 of any one of SEQ ID NOs: 6-69 or residues 7-153 of any one of SEQ ID NOs: 70-93, and wherein said second subunit comprises an amino acid sequence having at least 95% sequence identity to residues 7-153 of any one of SEQ ID NOs: 6-69 or residues 198-344 of any one of SEQ ID NOS: 70-93.

24. The method of any one of claims 19-23, wherein said P23H recognition sequence comprises SEQ ID NO:1 and said HVR1 region comprises W or Y at a position corresponding to:
(A) position 215 of any one of SEQ ID NOs: 6-69; or
(B) position 24 of any one of SEQ ID NOs: 70-93.

25. The method of any one of claims 19-24, wherein said P23H recognition sequence comprises SEQ ID NO:1 and said HVR1 region comprises residues 215-270 of any one of SEQ ID NOs: 6-69 or residues 24-79 of any one of SEQ ID NOs: 70-93.

26. The method of any one of claims 19-25, wherein said P23H recognition sequence comprises SEQ ID NO: 1 and said HVR2 region comprises residues 24-79 of any one of SEQ ID NOs: 6-69 or residues 215-270 of any one of SEQ ID NOs: 70-93.

27. The method of any one of claims 19-26, wherein said recombinant meganuclease is a single-chain meganuclease comprising a linker, wherein said linker covalently joins said first subunit and said second subunit.

28. The method of any one of claims 19-27, wherein said first subunit comprises residues 198-344 of any one of SEQ ID NOs: 6-69 or residues 7-153 of any one of SEQ ID NOs: 70-93.

29. The method of any one of claims 19-28, wherein said second subunit comprises residues 7-153 of any one of SEQ ID NOs: 6-69 or residues 198-344 of any one of SEQ ID NOs: 70-93.

30. The method of any one of claims 19-29, wherein said recombinant meganuclease comprises the amino acid sequence of any one of SEQ ID NOs: 6-93.

31. The recombinant meganuclease of any one of claims 19-30, wherein said P23H recognition sequence comprises one of SEQ ID NOs: 1-4.

32. The recombinant meganuclease of any one of claims 19-31, wherein said P23H recognition sequence comprises SEQ ID NO: 1.

33. The method of any one of claims 19-32, wherein said recombinant meganuclease preferentially recognizes and cleaves a P23H recognition sequence comprising SEQ ID NO: 1 relative to a recognition sequence comprising SEQ ID NO: 5.

34. The method of any one of claims 19-33, wherein said target cell in said subject is a retinal cell.

35. The method of any one of claims 19-34, wherein a gene encoding said recombinant meganuclease is delivered to said target cell using a recombinant AAV vector.
